SEQUENCE LISTING (361 sequences)

SEQ ID NO: 1 - protein, 6 aa, Artificial Sequence
  Monoclonal antibody segment
QSISSY

SEQ ID NO: 2 - protein, 3 aa, Artificial Sequence
  Monoclonal antibody segment
AAS

SEQ ID NO: 3 - protein, 9 aa, Artificial Sequence
  Monoclonal antibody segment
QQSYSTPLT

SEQ ID NO: 4 - protein, 6 aa, Artificial Sequence
  Monoclonal antibody segment
QSISRY

SEQ ID NO: 5 - protein, 9 aa, Artificial Sequence
  Monoclonal antibody segment
QQSYRTPYT

SEQ ID NO: 6 - protein, 9 aa, Artificial Sequence
  Monoclonal antibody segment
QQSYSTLGT

SEQ ID NO: 7 - protein, 6 aa, Artificial Sequence
  Monoclonal antibody segment
QSINNY

SEQ ID NO: 8 - protein, 9 aa, Artificial Sequence
  Monoclonal antibody segment
QQSYSIPLT

SEQ ID NO: 9 - protein, 8 aa, Artificial Sequence
  Monoclonal antibody segment
QSVNKNNY

SEQ ID NO: 10 - protein, 3 aa, Artificial Sequence
  Monoclonal antibody segment
SAS

SEQ ID NO: 11 - protein, 12 aa, Artificial Sequence
  Monoclonal antibody segment
QGTYYDSGWYNA

SEQ ID NO: 12 - protein, 6 aa, Artificial Sequence
  Monoclonal antibody segment
ESIGNA

SEQ ID NO: 13 - protein, 3 aa, Artificial Sequence
  Monoclonal antibody segment
KAS

SEQ ID NO: 14 - protein, 13 aa, Artificial Sequence
  Monoclonal antibody segment
QRYYFNGVSVEIV

SEQ ID NO: 15 - protein, 6 aa, Artificial Sequence
  Monoclonal antibody segment
EDIYSY

SEQ ID NO: 16 - protein, 3 aa, Artificial Sequence
  Monoclonal antibody segment
EAS

SEQ ID NO: 17 - protein, 14 aa, Artificial Sequence
  Monoclonal antibody segment
QNNFYMIGDDYGGT

SEQ ID NO: 18 - protein, 6 aa, Artificial Sequence
  Monoclonal antibody segment
QSIGSN

SEQ ID NO: 19 - protein, 3 aa, Artificial Sequence
  Monoclonal antibody segment
TAS

SEQ ID NO: 20 - protein, 14 aa, Artificial Sequence
  Monoclonal antibody segment
QCTYYGTSYMELGA

SEQ ID NO: 21 - protein, 6 aa, Artificial Sequence
  Monoclonal antibody segment
ESINDR

SEQ ID NO: 22 - protein, 3 aa, Artificial Sequence
  Monoclonal antibody segment
ETS

SEQ ID NO: 23 - protein, 12 aa, Artificial Sequence
  Monoclonal antibody segment
QSHDYISSSGMP

SEQ ID NO: 24 - protein, 8 aa, Artificial Sequence
  Monoclonal antibody segment
QSLYNKKN

SEQ ID NO: 25 - protein, 13 aa, Artificial Sequence
  Monoclonal antibody segment
QGEFSCVAADCFS

SEQ ID NO: 26 - protein, 6 aa, Artificial Sequence
  Monoclonal antibody segment
ESIYSY

SEQ ID NO: 27 - protein, 3 aa, Artificial Sequence
  Monoclonal antibody segment
YTS

SEQ ID NO: 28 - protein, 12 aa, Artificial Sequence
  Monoclonal antibody segment
QNTYGSSSNAFP

SEQ ID NO: 29 - protein, 6 aa, Artificial Sequence
  Monoclonal antibody segment
EDIYSG

SEQ ID NO: 30 - protein, 3 aa, Artificial Sequence
  Monoclonal antibody segment
RAS

SEQ ID NO: 31 - protein, 14 aa, Artificial Sequence
  Monoclonal antibody segment
QNNYGSSSNNYVFA

SEQ ID NO: 32 - protein, 7 aa, Artificial Sequence
  Monoclonal antibody segment
QSISSSY

SEQ ID NO: 33 - protein, 13 aa, Artificial Sequence
  Monoclonal antibody segment
LGGYFSFSNYDYG

SEQ ID NO: 34 - protein, 6 aa, Artificial Sequence
  Monoclonal antibody segment
QSIASR

SEQ ID NO: 35 - protein, 11 aa, Artificial Sequence
  Monoclonal antibody segment
QSYYGSGYGKT

SEQ ID NO: 36 - protein, 6 aa, Artificial Sequence
  Monoclonal antibody segment
ESISTY

SEQ ID NO: 37 - protein, 12 aa, Artificial Sequence
  Monoclonal antibody segment
QQGYTYKNVDNA

SEQ ID NO: 38 - protein, 6 aa, Artificial Sequence
  Monoclonal antibody segment
HNINSA

SEQ ID NO: 39 - protein, 13 aa, Artificial Sequence
  Monoclonal antibody segment
QRHYFDGASADIA

SEQ ID NO: 40 - protein, 6 aa, Artificial Sequence
  Monoclonal antibody segment
QSIRSY

SEQ ID NO: 41 - protein, 13 aa, Artificial Sequence
  Monoclonal antibody segment
QNVYYLSSSTYGA

SEQ ID NO: 42 - protein, 6 aa, Artificial Sequence
  Monoclonal antibody segment
ESISVR

SEQ ID NO: 43 - protein, 12 aa, Artificial Sequence
  Monoclonal antibody segment
RNYYDISNNGAG

SEQ ID NO: 44 - protein, 6 aa, Artificial Sequence
  Monoclonal antibody segment
QSIGSS

SEQ ID NO: 45 - protein, 12 aa, Artificial Sequence
  Monoclonal antibody segment
QNYISSSTFGND

SEQ ID NO: 46 - protein, 8 aa, Artificial Sequence
  Monoclonal antibody segment
ESVYKSNY

SEQ ID NO: 47 - protein, 3 aa, Artificial Sequence
  Monoclonal antibody segment
AAA

SEQ ID NO: 48 - protein, 12 aa, Artificial Sequence
  Monoclonal antibody segment
QGTYYSSGWYNA

SEQ ID NO: 49 - protein, 6 aa, Artificial Sequence
  Monoclonal antibody segment
ESISSW

SEQ ID NO: 50 - protein, 3 aa, Artificial Sequence
  Monoclonal antibody segment
EAF

SEQ ID NO: 51 - protein, 12 aa, Artificial Sequence
  Monoclonal antibody segment
QSAYYGGTYVGG

SEQ ID NO: 52 - protein, 6 aa, Artificial Sequence
  Monoclonal antibody segment
QNNNNY

SEQ ID NO: 53 - protein, 12 aa, Artificial Sequence
  Monoclonal antibody segment
QCTDYGPTYLGT

SEQ ID NO: 54 - protein, 3 aa, Artificial Sequence
  Monoclonal antibody segment
DAS

SEQ ID NO: 55 - protein, 12 aa, Artificial Sequence
  Monoclonal antibody segment
QQGDTYINVDNI

SEQ ID NO: 56 - protein, 6 aa, Artificial Sequence
  Monoclonal antibody segment
QSIDNY

SEQ ID NO: 57 - protein, 10 aa, Artificial Sequence
  Monoclonal antibody segment
QSYYGSSTNA

SEQ ID NO: 58 - protein, 6 aa, Artificial Sequence
  Monoclonal antibody segment
QSIYSS

SEQ ID NO: 59 - protein, 3 aa, Artificial Sequence
  Monoclonal antibody segment
GAS

SEQ ID NO: 60 - protein, 9 aa, Artificial Sequence
  Monoclonal antibody segment
QCSGYNSRV

SEQ ID NO: 61 - protein, 6 aa, Artificial Sequence
  Monoclonal antibody segment
QNIGSY

SEQ ID NO: 62 - protein, 6 aa, Artificial Sequence
  Monoclonal antibody segment
ESIGTA

SEQ ID NO: 63 - protein, 12 aa, Artificial Sequence
  Monoclonal antibody segment
QFTYYGNSYGNA

SEQ ID NO: 64 - protein, 7 aa, Artificial Sequence
  Monoclonal antibody segment
QNIYNNR

SEQ ID NO: 65 - protein, 3 aa, Artificial Sequence
  Monoclonal antibody segment
QAS

SEQ ID NO: 66 - protein, 10 aa, Artificial Sequence
  Monoclonal antibody segment
AGGYSGTIFT

SEQ ID NO: 67 - protein, 6 aa, Artificial Sequence
  Monoclonal antibody segment
ERIYNN

SEQ ID NO: 68 - protein, 12 aa, Artificial Sequence
  Monoclonal antibody segment
QCTGYGTTYGLV

SEQ ID NO: 69 - protein, 6 aa, Artificial Sequence
  Monoclonal antibody segment
QTISSY

SEQ ID NO: 70 - protein, 12 aa, Artificial Sequence
  Monoclonal antibody segment
QCPYYGSSHVGA

SEQ ID NO: 71 - protein, 3 aa, Artificial Sequence
  Monoclonal antibody segment
YAS

SEQ ID NO: 72 - protein, 12 aa, Artificial Sequence
  Monoclonal antibody segment
QQGARSSNVDNI

SEQ ID NO: 73 - protein, 8 aa, Artificial Sequence
  Monoclonal antibody segment
ESVYKNNY

SEQ ID NO: 74 - protein, 13 aa, Artificial Sequence
  Monoclonal antibody segment
AGYYGSSSVDRYT

SEQ ID NO: 75 - protein, 6 aa, Artificial Sequence
  Monoclonal antibody segment
QTINNA

SEQ ID NO: 76 - protein, 12 aa, Artificial Sequence
  Monoclonal antibody segment
AGYRPYINDGSA

SEQ ID NO: 77 - protein, 6 aa, Artificial Sequence
  Monoclonal antibody segment
QSIRTY

SEQ ID NO: 78 - protein, 12 aa, Artificial Sequence
  Monoclonal antibody segment
QQGYYWSDVENT

SEQ ID NO: 79 - protein, 8 aa, Artificial Sequence
  Monoclonal antibody segment
QSVDSNNY

SEQ ID NO: 80 - protein, 10 aa, Artificial Sequence
  Monoclonal antibody segment
AGGLGDNIYA

SEQ ID NO: 81 - protein, 8 aa, Artificial Sequence
  Monoclonal antibody segment
GYTFTGYY

SEQ ID NO: 82 - protein, 8 aa, Artificial Sequence
  Monoclonal antibody segment
INPNSGGT

SEQ ID NO: 83 - protein, 10 aa, Artificial Sequence
  Monoclonal antibody segment
AGGRIDAFDI

SEQ ID NO: 84 - protein, 13 aa, Artificial Sequence
  Monoclonal antibody segment
ARRYSSSLRAFDI

SEQ ID NO: 85 - protein, 8 aa, Artificial Sequence
  Monoclonal antibody segment
GFTVSSNY

SEQ ID NO: 86 - protein, 7 aa, Artificial Sequence
  Monoclonal antibody segment
IYSGGST

SEQ ID NO: 87 - protein, 13 aa, Artificial Sequence
  Monoclonal antibody segment
ARESAVAGWGMDV

SEQ ID NO: 88 - protein, 8 aa, Artificial Sequence
  Monoclonal antibody segment
GYSFTSYW

SEQ ID NO: 89 - protein, 8 aa, Artificial Sequence
  Monoclonal antibody segment
IYPGDSDT

SEQ ID NO: 90 - protein, 12 aa, Artificial Sequence
  Monoclonal antibody segment
ARGLAASTRFDP

SEQ ID NO: 91 - protein, 8 aa, Artificial Sequence
  Monoclonal antibody segment
GIDLSNYA

SEQ ID NO: 92 - protein, 7 aa, Artificial Sequence
  Monoclonal antibody segment
ISSGDVT

SEQ ID NO: 93 - protein, 11 aa, Artificial Sequence
  Monoclonal antibody segment
ARFFAGRSESL

SEQ ID NO: 94 - protein, 8 aa, Artificial Sequence
  Monoclonal antibody segment
GIDLTSGT

SEQ ID NO: 95 - protein, 7 aa, Artificial Sequence
  Monoclonal antibody segment
IRGSGST

SEQ ID NO: 96 - protein, 13 aa, Artificial Sequence
  Monoclonal antibody segment
ARAFPGYNTFFNF

SEQ ID NO: 97 - protein, 8 aa, Artificial Sequence
  Monoclonal antibody segment
GFSLSSYY

SEQ ID NO: 98 - protein, 7 aa, Artificial Sequence
  Monoclonal antibody segment
ISDRGST

SEQ ID NO: 99 - protein, 15 aa, Artificial Sequence
  Monoclonal antibody segment
YRGVDGSGGYAAGDL

SEQ ID NO: 100 - protein, 8 aa, Artificial Sequence
  Monoclonal antibody segment
GFSLSSYA

SEQ ID NO: 101 - protein, 7 aa, Artificial Sequence
  Monoclonal antibody segment
IDVTGRT

SEQ ID NO: 102 - protein, 16 aa, Artificial Sequence
  Monoclonal antibody segment
GRDSYLDYGDPNGLDP

SEQ ID NO: 103 - protein, 8 aa, Artificial Sequence
  Monoclonal antibody segment
TIDVSKYA

SEQ ID NO: 104 - protein, 7 aa, Artificial Sequence
  Monoclonal antibody segment
ISSSGST

SEQ ID NO: 105 - protein, 11 aa, Artificial Sequence
  Monoclonal antibody segment
ARGGNGVAFKL

SEQ ID NO: 106 - protein, 8 aa, Artificial Sequence
  Monoclonal antibody segment
GFSLGRDA

SEQ ID NO: 107 - protein, 7 aa, Artificial Sequence
  Monoclonal antibody segment
INDDGDT

SEQ ID NO: 108 - protein, 16 aa, Artificial Sequence
  Monoclonal antibody segment
ARVGAEYLGWGNGFNL

SEQ ID NO: 109 - protein, 9 aa, Artificial Sequence
  Monoclonal antibody segment
GFSFSSSQY

SEQ ID NO: 110 - protein, 9 aa, Artificial Sequence
  Monoclonal antibody segment
IYGGSSGLT

SEQ ID NO: 111 - protein, 13 aa, Artificial Sequence
  Monoclonal antibody segment
ARAIGPTYYAFDP

SEQ ID NO: 112 - protein, 8 aa, Artificial Sequence
  Monoclonal antibody segment
GFYLSTHG

SEQ ID NO: 113 - protein, 7 aa, Artificial Sequence
  Monoclonal antibody segment
IVTSGNT

SEQ ID NO: 114 - protein, 10 aa, Artificial Sequence
  Monoclonal antibody segment
ARPGYKSVPL

SEQ ID NO: 115 - protein, 8 aa, Artificial Sequence
  Monoclonal antibody segment
GIDLSSYA

SEQ ID NO: 116 - protein, 7 aa, Artificial Sequence
  Monoclonal antibody segment
IGASGTT

SEQ ID NO: 117 - protein, 16 aa, Artificial Sequence
  Monoclonal antibody segment
ARAGYAGTGYPTRLDL

SEQ ID NO: 118 - protein, 8 aa, Artificial Sequence
  Monoclonal antibody segment
GFDFSSNT

SEQ ID NO: 119 - protein, 7 aa, Artificial Sequence
  Monoclonal antibody segment
ISYSGAT

SEQ ID NO: 120 - protein, 17 aa, Artificial Sequence
  Monoclonal antibody segment
ARDRGYVDGSYYGDFNL

SEQ ID NO: 121 - protein, 8 aa, Artificial Sequence
  Monoclonal antibody segment
GFSLSSYP

SEQ ID NO: 122 - protein, 7 aa, Artificial Sequence
  Monoclonal antibody segment
ITSGGSI

SEQ ID NO: 123 - protein, 14 aa, Artificial Sequence
  Monoclonal antibody segment
ARGYSSGTMMSSNL

SEQ ID NO: 124 - protein, 8 aa, Artificial Sequence
  Monoclonal antibody segment
GIDLSSNT

SEQ ID NO: 125 - protein, 7 aa, Artificial Sequence
  Monoclonal antibody segment
MRGGGST

SEQ ID NO: 126 - protein, 13 aa, Artificial Sequence
  Monoclonal antibody segment
ARAFPGYNSFFNF

SEQ ID NO: 127 - protein, 9 aa, Artificial Sequence
  Monoclonal antibody segment
GFSFSGGYN

SEQ ID NO: 128 - protein, 9 aa, Artificial Sequence
  Monoclonal antibody segment
IYAGSSGST

SEQ ID NO: 129 - protein, 16 aa, Artificial Sequence
  Monoclonal antibody segment
ARGAYAGGSYPPYFNL

SEQ ID NO: 130 - protein, 8 aa, Artificial Sequence
  Monoclonal antibody segment
GFSLSNYA

SEQ ID NO: 131 - protein, 7 aa, Artificial Sequence
  Monoclonal antibody segment
IVTSGST

SEQ ID NO: 132 - protein, 11 aa, Artificial Sequence
  Monoclonal antibody segment
ARHAYSIGSSL

SEQ ID NO: 133 - protein, 13 aa, Artificial Sequence
  Monoclonal antibody segment
ARGSGGAVTYYGV

SEQ ID NO: 134 - protein, 7 aa, Artificial Sequence
  Monoclonal antibody segment
ISSSDNT

SEQ ID NO: 135 - protein, 11 aa, Artificial Sequence
  Monoclonal antibody segment
ARFYAGRSYSL

SEQ ID NO: 136 - protein, 8 aa, Artificial Sequence
  Monoclonal antibody segment
GFSLSTYY

SEQ ID NO: 137 - protein, 8 aa, Artificial Sequence
  Monoclonal antibody segment
IRGGGEPV

SEQ ID NO: 138 - protein, 14 aa, Artificial Sequence
  Monoclonal antibody segment
ARRLDGFVRAYLDL

SEQ ID NO: 139 - protein, 7 aa, Artificial Sequence
  Monoclonal antibody segment
SGYPGRT

SEQ ID NO: 140 - protein, 12 aa, Artificial Sequence
  Monoclonal antibody segment
ARHYGSTNGLNL

SEQ ID NO: 141 - protein, 9 aa, Artificial Sequence
  Monoclonal antibody segment
GFSFSSYYD

SEQ ID NO: 142 - protein, 9 aa, Artificial Sequence
  Monoclonal antibody segment
IYIGNSGNT

SEQ ID NO: 143 - protein, 15 aa, Artificial Sequence
  Monoclonal antibody segment
ARGASGYSGYGWFIL

SEQ ID NO: 144 - protein, 8 aa, Artificial Sequence
  Monoclonal antibody segment
GIDLSNYI

SEQ ID NO: 145 - protein, 7 aa, Artificial Sequence
  Monoclonal antibody segment
IASNGGT

SEQ ID NO: 146 - protein, 13 aa, Artificial Sequence
  Monoclonal antibody segment
ARGAGTTWSSFNL

SEQ ID NO: 147 - protein, 7 aa, Artificial Sequence
  Monoclonal antibody segment
IYASGTT

SEQ ID NO: 148 - protein, 10 aa, Artificial Sequence
  Monoclonal antibody segment
ARDSYSTGNL

SEQ ID NO: 149 - protein, 9 aa, Artificial Sequence
  Monoclonal antibody segment
GFSFSSGYW

SEQ ID NO: 150 - protein, 9 aa, Artificial Sequence
  Monoclonal antibody segment
IGARDSGSA

SEQ ID NO: 151 - protein, 11 aa, Artificial Sequence
  Monoclonal antibody segment
ARSGASFHFKL

SEQ ID NO: 152 - protein, 8 aa, Artificial Sequence
  Monoclonal antibody segment
GFSLSTYN

SEQ ID NO: 153 - protein, 7 aa, Artificial Sequence
  Monoclonal antibody segment
INGGGSA

SEQ ID NO: 154 - protein, 16 aa, Artificial Sequence
  Monoclonal antibody segment
ARGYYAGADWNMPLDL

SEQ ID NO: 155 - protein, 9 aa, Artificial Sequence
  Monoclonal antibody segment
GFSFSGGYD

SEQ ID NO: 156 - protein, 16 aa, Artificial Sequence
  Monoclonal antibody segment
ARGAYAGGGYPPYFNF

SEQ ID NO: 157 - protein, 8 aa, Artificial Sequence
  Monoclonal antibody segment
GFSLSSSG

SEQ ID NO: 158 - protein, 7 aa, Artificial Sequence
  Monoclonal antibody segment
TFPSGSA

SEQ ID NO: 159 - protein, 11 aa, Artificial Sequence
  Monoclonal antibody segment
AHVGATTGRDF

SEQ ID NO: 160 - protein, 8 aa, Artificial Sequence
  Monoclonal antibody segment
GIDLSDYY

SEQ ID NO: 161 - protein, 7 aa, Artificial Sequence
  Monoclonal antibody segment
IYASGNA

SEQ ID NO: 162 - protein, 10 aa, Artificial Sequence
  Monoclonal antibody segment
ARNIGGSSNL

SEQ ID NO: 163 - protein, 9 aa, Artificial Sequence
  Monoclonal antibody segment
GFSLSKNYV

SEQ ID NO: 164 - protein, 7 aa, Artificial Sequence
  Monoclonal antibody segment
IYAGDGN

SEQ ID NO: 165 - protein, 11 aa, Artificial Sequence
  Monoclonal antibody segment
ARWDYIGNNNL

SEQ ID NO: 166 - protein, 8 aa, Artificial Sequence
  Monoclonal antibody segment
GIDLISYP

SEQ ID NO: 167 - protein, 7 aa, Artificial Sequence
  Monoclonal antibody segment
IATGGST

SEQ ID NO: 168 - protein, 10 aa, Artificial Sequence
  Monoclonal antibody segment
ARRWSSAYDF

SEQ ID NO: 169 - protein, 8 aa, Artificial Sequence
  Monoclonal antibody segment
GIDLSSYT

SEQ ID NO: 170 - protein, 7 aa, Artificial Sequence
  Monoclonal antibody segment
IAINGGT

SEQ ID NO: 171 - protein, 13 aa, Artificial Sequence
  Monoclonal antibody segment
ARGAGKTWSSFNL

SEQ ID NO: 172 - protein, 8 aa, Artificial Sequence
  Monoclonal antibody segment
GIDLSRYS

SEQ ID NO: 173 - protein, 7 aa, Artificial Sequence
  Monoclonal antibody segment
MSIGSSA

SEQ ID NO: 174 - protein, 12 aa, Artificial Sequence
  Monoclonal antibody segment
ARSNLYRGAFDP

SEQ ID NO: 175 - protein, 9 aa, Artificial Sequence
  Monoclonal antibody segment
GFSFSSSQD

SEQ ID NO: 176 - protein, 8 aa, Artificial Sequence
  Monoclonal antibody segment
IGFGSGNT

SEQ ID NO: 177 - protein, 14 aa, Artificial Sequence
  Monoclonal antibody segment
ARDPFTVAGDGDNL

SEQ ID NO: 178 - protein, 8 aa, Artificial Sequence
  Monoclonal antibody segment
GFSINDFH

SEQ ID NO: 179 - protein, 7 aa, Artificial Sequence
  Monoclonal antibody segment
IGTGGSA

SEQ ID NO: 180 - protein, 13 aa, Artificial Sequence
  Monoclonal antibody segment
ARRDWSDGGYSLL

SEQ ID NO: 181 - protein, 8 aa, Artificial Sequence
  Monoclonal antibody segment
GFSLSINA

SEQ ID NO: 182 - protein, 7 aa, Artificial Sequence
  Monoclonal antibody segment
ITNIGNT

SEQ ID NO: 183 - protein, 8 aa, Artificial Sequence
  Monoclonal antibody segment
ARNSYFDP

SEQ ID NO: 184 - protein, 117 aa, Artificial Sequence
  Monoclonal antibody segment
EVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAGGRIDAFDIWGQGTTVSVSS

SEQ ID NO: 185 - protein, 107 aa, Artificial Sequence
  Monoclonal antibody segment
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 186 - protein, 120 aa, Artificial Sequence
  Monoclonal antibody segment
  misc_feature (87)..(87): Xaa can be any naturally occurring amino acid
EVQLVQSGAEVKKPGASVKVSCKASGYTFTGYYMHWVRQAPGQGLEWMGRINPNSGGTNYAQKFQGRVTMTRDTSISTAYMELSRLXSDDTAVYYCARRYSSSLRAFDIWGQGTMVSVSS

SEQ ID NO: 187 - protein, 107 aa, Artificial Sequence
  Monoclonal antibody segment
DIQMTQSPSSLSASVGDRVSITCRASQSISRYLNWYQQKPGKAPKLLIYAASSLQSGVPSRFSGSGSGTDFTLTISSLQPEDFATYYCQQSYRTPYTFSQGTKLEIK

SEQ ID NO: 188 - protein, 119 aa, Artificial Sequence
  Monoclonal antibody segment
EVQLVQSGGGLVQPGGSLRLSCAASGFTVSSNYMSWVRQAPGKGLEWVSVIYSGGSTYYADSVKGRFTISRHNSKNTLYLQMNSLRAEDTAVYYCARESAVAGWGMDVWGKGTTVTVSS

SEQ ID NO: 189 - protein, 107 aa, Artificial Sequence
  Monoclonal antibody segment
DIQMTQSPSSLSASVGDRVTITCRASQSISSYLNWYQQKPGKAPKLLIYAASSLQSGVPSRFSGSGSGTDFTLTISSLQPEDFATYYCQQSYSTLGTFGQGTKLEIK

SEQ ID NO: 190 - protein, 119 aa, Artificial Sequence
  Monoclonal antibody segment
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGLAASTRFDPWGQGTLVTVSS

SEQ ID NO: 191 - protein, 107 aa, Artificial Sequence
  Monoclonal antibody segment
DVQMTQSPSSLSASVGDRVTITCRPSQSINNYLNWYQQKPGEAPKLLIYAASNLQSGVPSRFSGSGSGTDFTLTISSLQPEDFGTYYCQQSYSIPLTFGGGTTVEIK

SEQ ID NO: 192 - protein, 115 aa, Artificial Sequence
  Monoclonal antibody segment
QSVEESGGRLVTPGTPLTLTCTVSGIDLSNYAINWVRQAPGKGLEWIGMISSGDVTYYASWAKGRFIISRTSSTTVDLKMTSPTIEDTATYFCARFFAGRSESLWGQGTLVTVSS

SEQ ID NO: 193 - protein, 112 aa, Artificial Sequence
  Monoclonal antibody segment
ELDLTQTPSSVSAAVGGTVTINCQSSQSVNKNNYLSWYQQKPGQPPKLLIYSASALASGVSSRFSGSGSGTEFTLTISDVQCDDAATYYCQGTYYDSGWYNAFGGGTEVEIK

SEQ ID NO: 194 - protein, 116 aa, Artificial Sequence
  Monoclonal antibody segment
QSLKESGGRLVKPDETLTITCTASGIDLTSGTVGWFRQAPGKGLEWIGIIRGSGSTYYATWAKGRFTISKTSTTVDLKLTSPTTEDTATYLCARAFPGYNTFFNFWGQGTLVTISS

SEQ ID NO: 195 - protein, 112 aa, Artificial Sequence
  Monoclonal antibody segment
ELDLTQTPSSVEAAVGGTVTIKCQASESIGNALAWYQQKPGQPPKLLIWKASNLESGVPPRFKGSGSGTEFTLTISDLECADAATYYCQRYYFNGVSVEIVFGGGTEVVVKG

SEQ ID NO: 196 - protein, 119 aa, Artificial Sequence
  Monoclonal antibody segment
QSVEESGGRLVTPGTPLTLTCTASGFSLSSYYMTWVRQAPGKGLEYIGIISDRGSTYYASWAKGRFTIAKTSSTTVDLKLTSLTTEDTATYFCYRGVDGSGGYAAGDLWGQGTLVTISS

SEQ ID NO: 197 - protein, 112 aa, Artificial Sequence
  Monoclonal antibody segment
ELVLTQTPASVEAAVGGTVTIKCQASEDIYSYFAWYQQKPGQRPRLLIYEASKVASGVPSRFKGGGSGTQFTLTISDLECADAATYYCQNNFYMIGDDYGGTFGGGTEVEIK

SEQ ID NO: 198 - protein, 119 aa, Artificial Sequence
  Monoclonal antibody segment
QSVKESGGRLVTPGTPLTLTCTVSGFSLSSYAMSWVRQAPGKGLEWIGVIDVTGRTYYASWAKGRFTISKTSTTVDLKITSPTTEDTATYFCGRDSYLDYGDPNGLDPWGPGTLVTISS

SEQ ID NO: 199 - protein, 112 aa, Artificial Sequence
  Monoclonal antibody segment
ELVMTQTPASVEAAVGGTVTIKCQASQSIGSNLAWYQQKPGQPPKLLIYTASNLAPGVSSRFKGSRSGTESTLIISDLECADAATYYCQCTYYGTSYMELGAFGGGTELEIK

SEQ ID NO: 200 - protein, 115 aa, Artificial Sequence
  Monoclonal antibody segment
QSLEESGGRLVTPGTPLTLTCTVSTIDVSKYAVIWVRQAPGKGLEYIGIISSSGSTYYASWAKGRFTISKTSSTTVDLKMTSPTAEDTATYFCARGGNGVAFKLWGQGTLVTISS

SEQ ID NO: 201 - protein, 110 aa, Artificial Sequence
  Monoclonal antibody segment
ELDMTQTPASVEAAVGGTVTINCQASESINDRLAWYQLKPGQPPKLLIYETSQVESGVPSRFSGSGSGTQFTLTISDLECADAATYYCQSHDYISSSGMPFGGGTEVVVK

SEQ ID NO: 202 - protein, 120 aa, Artificial Sequence
  Monoclonal antibody segment
QSVKESGGRLVTPGTPLTLTCTMSGFSLGRDALTWVRQAPGKGLQWIAFINDDGDTDYPNWARGRFTISKTSSTTVDLKITSPTTEDTATYFCARVGAEYLGWGNGFNLWGQGTLVTISS

SEQ ID NO: 203 - protein, 113 aa, Artificial Sequence
  Monoclonal antibody segment
ELDMTQTPASVSAAVGGTVTINCQASQSLYNKKNLAWYQQKPGQPPKLLIYKASTLASGVPSRFEGSGSGTEYALTISGLQCDDAATYYCQGEFSCVAADCFSFGGGTEVVVK

SEQ ID NO: 204 - protein, 120 aa, Artificial Sequence
  Monoclonal antibody segment
QSVKESGGDLVKPGASLTLTCTASGFSFSSSQYMCWVRQAPGKGLEWIACIYGGSSGLTNYATWAKGRFTVSKTSSTTVTLQMTSLTDADTATYFCARAIGPTYYAFDPWGPGTLVTVSS

SEQ ID NO: 205 - protein, 110 aa, Artificial Sequence
  Monoclonal antibody segment
ELDLTQTPSSVSAAVGDTVTIKCQASESIYSYLAWYQQKPGQPPKLLIYYTSTLASGVPSRFKGSGSGTEFTLTISDLECADAATYYCQNTYGSSSNAFPFGGGTEVEIK

SEQ ID NO: 206 - protein, 113 aa, Artificial Sequence
  Monoclonal antibody segment
QSVKESGGRLVTPGTPLTLTCTVSGFYLSTHGMTWVRQAPGKGLEYIGVIVTSGNTYYATWATGRFTISKTSTTVDLKITSPTTEDTATYFCARPGYKSVPLWGQGTLVTISS

SEQ ID NO: 207 - protein, 112 aa, Artificial Sequence
  Monoclonal antibody segment
ELDLTQTPASVSAAVGGTVTINCQASEDIYSGLTWYQQKPGQPPKLLIYRASTLASGVPPRFKGSGSGTEFTLTISDLECDDAATYYCQNNYGSSSNNYVFAFGGGTEVEIK

SEQ ID NO: 208 - protein, 119 aa, Artificial Sequence
  Monoclonal antibody segment
QSVKESGGRLVTPGTPLTLTCTLSGIDLSSYAMGWVRQAPGKGLEYIGIIGASGTTYYANWAKGRFTISRTSTTVDLKITSPTTEDTATYFCARAGYAGTGYPTRLDLWGQGTLVTISS

SEQ ID NO: 209 - protein, 112 aa, Artificial Sequence
  Monoclonal antibody segment
ELDMTQTPASVEAAVGGTVTIKCQASQSISSSYLAWYQQKPGQPPKLLIYKASTLESGVPSRFSGSGSGTEFTLTISGVQCDDAATYYCLGGYFSFSNYDYGFGGGTEVEIK

SEQ ID NO: 210 - protein, 121 aa, Artificial Sequence
  Monoclonal antibody segment
QSVKESGGDLVKPGASLTLTCTASGFDFSSNTMCWVRQAPGKGLEYIGYISYSGATHYASWAKGRFTISKTLSTTVTLQMTSLTDADTATYFCARDRGYVDGSYYGDFNLWGPGTLVTVSS

SEQ ID NO: 211 - protein, 109 aa, Artificial Sequence
  Monoclonal antibody segment
ELVMTQTPSSASEPVGGTVTIKCQASQSIASRLAWFQQKPGQPPKLLIYRASTLESGVSSRFKGSGFGTEFTLTISDLECADAATYYCQSYYGSGYGKTFGGGTDVVVK

SEQ ID NO: 212 - protein, 117 aa, Artificial Sequence
  Monoclonal antibody segment
QSLEESGGRLVTPGTPLTLTCTVSGFSLSSYPMSWVRQAPGKGLEWIGIITSGGSIFYATWANGRFTISKTSTTVDLKITSPTTEDTATYFCARGYSSGTMMSSNLWGQGTLVTVSS

SEQ ID NO: 213 - protein, 110 aa, Artificial Sequence
  Monoclonal antibody segment
ELVLTQTPASVSEPVGGTVTIKCQASESISTYLSWYQQKPGQRPKLLIYRASTLESGVPSRFKGSGSGTEYTLTISGVQCADAATYYCQQGYTYKNVDNAFGGGTEVVVK

SEQ ID NO: 214 - protein, 116 aa, Artificial Sequence
  Monoclonal antibody segment
QSLEESGGRLVMPGGSLTLTCTVSGIDLSSNTMGWFRQAPGKGLEWIGIMRGGGSTYYATWAKGRFTISKTSTTVDLKLTSPTTEDTATYFCARAFPGYNSFFNFWGQGTLVTISS

SEQ ID NO: 215 - protein, 107 aa, Artificial Sequence
  Monoclonal antibody segment
TQTPASVSEPVGGTVTIKCQASHNINSALAWYQQKPGQPPKLLIYKASTLASGVPPRFKGSGSGTEFTLTISDLECADAATYYCQRHYFDGASADIAFGGGTEVEIK

SEQ ID NO: 216 - protein, 123 aa, Artificial Sequence
  Monoclonal antibody segment
  misc_feature (4)..(4): Xaa can be any naturally occurring amino acid
QSVXESGGGLVQPEGSLTLTCTASGFSFSGGYNMCWVRQAPGKGLEWIACIYAGSSGSTYYASWAKGRFTISKTSSTTVTLQMTSLTAADTATYFCARGAYAGGSYPPYFNLWGPGTLVTISS

SEQ ID NO: 217 - protein, 111 aa, Artificial Sequence
  Monoclonal antibody segment
  misc_feature (2)..(4): Xaa can be any naturally occurring amino acid
EXXXTQTPASVSEPVGGTVTIKCQASQSIRSYLAWYQQKAGQPPKLLIYRASTLASGVPSRFKGSGSGTEYTLTISDLECADAATYYCQNVYYLSSSTYGAFGGGTEVVVK

SEQ ID NO: 218 - protein, 114 aa, Artificial Sequence
  Monoclonal antibody segment
QSVKESGGRLVTPGTPLTLTCTVSGFSLSNYAMSWVRQAPGKGLEWIGIIVTSGSTYYASWAKGRFTISKTSTTVDLKMTSLTAADTATYFCARHAYSIGSSLWGQGTLVTISS

SEQ ID NO: 219 - protein, 110 aa, Artificial Sequence
  Monoclonal antibody segment
ELDLTQTPPSASEPVGGTVTIKCQASESISVRLAWYQQKPGQPPKLLIYKASNLPSGVPSRFKGSGSGTEFTLTISDLECDDAATYYCRNYYDISNNGAGFGGGTEVEIK

SEQ ID NO: 220 - protein, 117 aa, Artificial Sequence
  Monoclonal antibody segment
QSVKESGGGLFKPTDTLTLTCTVSGIDLSSYAMTWVRQAPGEGLEWIGTISSSGSTYYASWAKGRFTISKTSSTTVDLKMTSLTAADTATYFCARGSGGAVTYYGVWGQGTLVTVSS

SEQ ID NO: 221 - protein, 110 aa, Artificial Sequence
  Monoclonal antibody segment
ELDLTQTPASVSEPVGGTVTIKCQASQSIGSSLAWYQQKPGQPPKLLIYRASTLASGVPSRFSGSGSGTEFTLTISDLECAHAATYYCQNYISSSTFGNDFGGGTEVVVK

SEQ ID NO: 222 - protein, 114 aa, Artificial Sequence
  Monoclonal antibody segment
QSVEESGGRLVTPGTPLTLTCTVSGIDLSSYAINWVRQAPGEGLEWIGMISSSDNTYYANWAKGRFTISRDSTTVDLKITSPTTEDTATYFCARFYAGRSYSLWGQGTLVTISS

SEQ ID NO: 223 - protein, 112 aa, Artificial Sequence
  Monoclonal antibody segment
ELDLTQTPASVSAAVGGTVTISCQSSESVYKSNYLSWYQQKAGQPPKLLIYAAATLASGVPSRFKGSGSGTQFTLTISGVQCDDAATYYCQGTYYSSGWYNAFGGGTEVVVK

SEQ ID NO: 224 - protein, 119 aa, Artificial Sequence
  Monoclonal antibody segment
EQSVEESGGRLVTPGTPLTLTCTASGFSLSTYYMSWVRQAPGKGLEWIGIIRGGGEPVWYADWAKGRFTISRTSTTVDLKITSPTPEDTATYFCARRLDGFVRAYLDLWGQGTLVTVSS

SEQ ID NO: 225 - protein, 106 aa, Artificial Sequence
  Monoclonal antibody segment
TQTPASVEAAVGGTVTIKCQASESISSWLAWYQQKPGQPPKLLIYEAFSLASGVPSRFSGSGSGTEFTLTISDLECDDAAIYYCQSAYYGGTYVGGFGGGTEVVVK

SEQ ID NO: 226 - protein, 116 aa, Artificial Sequence
  Monoclonal antibody segment
QSVEESGGRLVMPGGSLTLTCTVSGIDLSSNTMGWFRQAPGKGLEWIGIMRGGGSTYYATWAKGRFTISKTSTTVDLKLTSPTTEDTATYFCARAFPGYNSFFNFWGQGTLVTISS

SEQ ID NO: 227 - protein, 111 aa, Artificial Sequence
  Monoclonal antibody segment
ELDLTQTPASVSEPVGGTVTIKCQASHNINSALAWYQQKPGQPPKLLIYKASTLASGVPPRFKGSGSGTEFTLTISDLECADAATYYCQRHYFDGASADIAFGGGTEVVVK

SEQ ID NO: 228 - protein, 115 aa, Artificial Sequence
  Monoclonal antibody segment
AAVKESGGRLVTPGTPLTLTCTVSGFSLSNYAMGWVRQPPGKGLEWIGFSGYPGRTYYASWAKGRFTISKTSTTVDLKITSPTAEDTATYFCARHYGSTNGLNLWGQGTLVTVSS

SEQ ID NO: 229 - protein, 110 aa, Artificial Sequence
  Monoclonal antibody segment
ELDLTQTPASVSEPVGGTVTIKCQASQNNNNYLAWYQQKPGQPPKLLIYAASSLASGVPSRFKGSGSGTQFTLTISDLECDDAATYYCQCTDYGPTYLGTFGGGTEVEIK

SEQ ID NO: 230 - protein, 122 aa, Artificial Sequence
  Monoclonal antibody segment
QSLEESGGDLVKPGASLTLTCTASGFSFSSYYDMCWVRQAPGKGLEWIACIYIGNSGNTYYASWAKGRFTISRTSSTTVDLKMSSLTAADTASYFCARGASGYSGYGWFILWGPGTLVTISS

SEQ ID NO: 231 - protein, 110 aa, Artificial Sequence
  Monoclonal antibody segment
ELVMTQTPASVSAAVGGTVTIKCQASQSIGSNLAWYQQKPGQRPKLLIYDASDLASGVPSRFKGSGSGPEYTLTISGVECDDAATYYCQQGDTYINVDNIFGGGTEVEIK

SEQ ID NO: 232 - protein, 116 aa, Artificial Sequence
  Monoclonal antibody segment
QSVEESGGRLVTPGTPLTLTCTVSGIDLSNYIMGWVRQAPGKGLEWIGIIASNGGTVYANWAKGRFTISKTSTTVDLKMTSPTPEDTATYFCARGAGTTWSSFNLWGQGTLVTISS

SEQ ID NO: 233 - protein, 108 aa, Artificial Sequence
  Monoclonal antibody segment
ELVLTQTPASVSEPVGGTVTINCQVSQSIDNYLNWYHQKPGQPPKRLIYRASTLASGVPSRFRGSGSGTQFTLTISDLECADAATYYCQSYYGSSTNAFGGGTEVVVK

SEQ ID NO: 234 - protein, 113 aa, Artificial Sequence
  Monoclonal antibody segment
QSVEESGGRLVTPGTPLTLTCTVSGFSLSSYAMNWVRQAPGKGLEWIGLIYASGTTHYASWVNGRFTISKTSTTVTLKMTSLTAADTATYFCARDSYSTGNLWGQGTLVTISS

SEQ ID NO: 235 - protein, 107 aa, Artificial Sequence
  Monoclonal antibody segment
ELVLTQTPASVSEPVGGTVTIKCQASQSIYSSLVWYQQKPGQPPKLLIYGASNLESGVPSRFKGSGSGTEYTLTISGVQCADAATYYCQCSGYNSRVFGGGTEVEIK

SEQ ID NO: 236 - protein, 118 aa, Artificial Sequence
  Monoclonal antibody segment
SSVEESGGGLVKPGASLTLTCTASGFSFSSGYWMCWVRQAPGKELEWIACIGARDSGSAYYATWAKGRFTISKISSTTVTLQMTSLTAADTATYFCARSGASFHFKLWGPGTLVTISS

SEQ ID NO: 237 - protein, 107 aa, Artificial Sequence
  Monoclonal antibody segment
ELVLTQTPASVSEPVGGTVTIKCQASQNIGSYLAWYQQKPGQPPKLLIYGASNLESGVPSRFKGSGSGTEYTLTISGVQCADAATYYCQCSGYNSRVFGGGTEVVVK

SEQ ID NO: 238 - protein, 119 aa, Artificial Sequence
  Monoclonal antibody segment
QSLEESGGDLVTPGTPLTLTCTVSGFSLSTYNIQWVRQAPGKGLECIGFINGGGSAYYASWVNGRFTVSKASTTVTLKITAPTTEDTATYFCARGYYAGADWNMPLDLWGQGTLVTVSS

SEQ ID NO: 239 - protein, 110 aa, Artificial Sequence
  Monoclonal antibody segment
ELVLTQTPSPVSAGVGGTVTIKCQSSESIGTALAWYQQKLGQPPKLLIYAAALLASGVSSRFKGSGSGTEYTLTISDLECDDAATYYCQFTYYGNSYGNAFGGGTEVEIK

SEQ ID NO: 240 - protein, 123 aa, Artificial Sequence
  Monoclonal antibody segment
QSVKESGGDLVKPEGSLTLTCTASGFSFSGGYDMCWVRQAPGKGLEWIACIYAGSSGSTYYASWAKGRFTISKTSSTTVTLQMTSLTAADTATYFCARGAYAGGGYPPYFNFWGPGTLVTISS

SEQ ID NO: 241 - protein, 110 aa, Artificial Sequence
  Monoclonal antibody segment
ELVMTQTPASVEAAVGGTVTINCQASESINDRLAWYQLKPGQPPKLLIYETSKVESGVPSRFSGSGSGTQFTLTISDLECADAATYYCQSHDYISSSGMPFGGGTELEIK

SEQ ID NO: 242 - protein, 114 aa, Artificial Sequence
  Monoclonal antibody segment
QSVKESGGRLVTPGTPLTLTCTVSGFSLSSSGMSWVRQAPGKGLEWIGLTFPSGSASYASWAKGRFTVSRTSTTVDLNLTSPTTEDTATYFCAHVGATTGRDFWGQGTLVTVSS

SEQ ID NO: 243 - protein, 109 aa, Artificial Sequence
  Monoclonal antibody segment
ELVMTQTASSVSAAVGGTVTISCQSSQNIYNNRWSWYQQKPGQPPKLLIYQASTLASGVPSRFKGSGSGTQFTLTISGVQCDDAATYYCAGGYSGTIFTFGGGTELEIK

SEQ ID NO: 244 - protein, 114 aa, Artificial Sequence
  Monoclonal antibody segment
AAVKESGGRLVTPGTPLTLTCTVSGIDLSDYYMTWVRQAPGKGLEYIGIIYASGNAYFASWAKGRFTISRTSSTTVDLNVASPTTEDTATYFCARNIGGSSNLWGQGTLVTVSS

SEQ ID NO: 245 - protein, 109 aa, Artificial Sequence
  Monoclonal antibody segment
ELDMTQTPASVSEPVGGTVTIKCQASERIYNNLAWYQQKPGQPPKLLFYSASTLASGVPSRFKGSGSGTEFTLTISDLECADAAIYYCQCTGYGTTYGLVFGGGTEVEI

SEQ ID NO: 246 - protein, 117 aa, Artificial Sequence
  Monoclonal antibody segment
QSVKESGGGLFQPGGSLALTCKASGFSLSKNYVMCWVRQAPGKGLEWIACIYAGDGNTYYVSWAKGRFTISKTSSTTVTLQMTSLTAADTATYFCARWDYIGNNNLWGPGTLVTISS

SEQ ID NO: 247 - protein, 110 aa, Artificial Sequence
  Monoclonal antibody segment
ELVLTQTPASVEAAVGGTVTIKCQASQTISSYLAWYQQKPGQPPKLLIYRASTLASGVPSRFKGSGSGTQFTLTISDLECADGATYYCQCPYYGSSHVGAFGGGTEVVVK

SEQ ID NO: 248 - protein, 113 aa, Artificial Sequence
  Monoclonal antibody segment
QSVKESGGRLVTPGTPLTLTCTVSGIDLISYPMGWVRQAPGKGLEYIGIIATGGSTYYASWAKGRFTISTSSTTVDLKMTSLTAADTATYFCARRWSSAYDFWGQGTLVTISS

SEQ ID NO: 249 - protein, 110 aa, Artificial Sequence
  Monoclonal antibody segment
ELDLTQTPASVEVAVGGTVTIKCQASQSISSYLAWYQQKPGQPPKLLIYYASTLASGVPSRFSGSGSGTEFTLTISGVQCDDAATYYCQQGARSSNVDNIFGGGTEVVVK

SEQ ID NO: 250 - protein, 116 aa, Artificial Sequence
  Monoclonal antibody segment
QSLEESGGRLVTPGGSLTLTCTVSGIDLSSYTMGWVRQAPGKGLEWIGVIAINGGTVYASWAKGRFTISKTSTTVDLKMTSPTPEDTATYFCARGAGKTWSSFNLWGQGTLVTISS

SEQ ID NO: 251 - protein, 108 aa, Artificial Sequence
  Monoclonal antibody segment
ELVLTQTPASVSEPVGGTVTIKCQASQSIGSNLAWYQQKPGQPPKLLIYRASTLESGVPSRFSGSGSGTEFTLTISDLECADAATYYCQSYYGSSTNAFGGGTEVVVK

SEQ ID NO: 252 - protein, 116 aa, Artificial Sequence
  Monoclonal antibody segment
QSVKESGGRLVKPDETLTITCTVSGIDLSRYSIQWVRQAPGGGLQWIGVMSIGSSAYYANWAKGRFTISKTSSTTVDLTMTSLTAADTATYFCARSNLYRGAFDPWGPGTLVTISS

SEQ ID NO: 253 - protein, 113 aa, Artificial Sequence
  Monoclonal antibody segment
ELVMTQTPASVEAAVGGTVTIKCQSSESVYKNNYLSWYQLKPGQPPKLLFYSASTLASRVPSRFSGSGSGTQFTLTISDLECDDAATYYCAGYYGSSSVDRYTFGGGTEVEIK

SEQ ID NO: 254 - protein, 120 aa, Artificial Sequence
  Monoclonal antibody segment
QLMESGGGLVQPGASLTLTCKASGFSFSSSQDMCWVRQAPGKGPEWIGCIGFGSGNTWYASWVNGRFTISRSTSLNTVELKMTGLTAADTATYFCARDPFTVAGDGDNLWGPGTLVTVSS

SEQ ID NO: 255 - protein, 110 aa, Artificial Sequence
  Monoclonal antibody segment
ELDLTQTPASVSEPVGGTVTIKCQASQTINNALAWYQQKPGQPPKLLIYSASTLASGVSSRFSGSGSGTQFTLTISGVECDDAATYYCAGYRPYINDGSAFGGGTEVVVK

SEQ ID NO: 256 - protein, 119 aa, Artificial Sequence
  Monoclonal antibody segment
QSVEESGGRLVTPGTPLTLTCTVSGFSLSSYAMSWVRQAPGKGLEWIGVIDVTGRTYYASWAKGRFTISKTSTTVDLKITSPTTEDTATYFCGRDSYLDYGDPNGLDPWGPGTLVTISS

SEQ ID NO: 257 - protein, 112 aa, Artificial Sequence
  Monoclonal antibody segment
ELVMTQTPASVEAAVGGTVTIKCQASQSIGSNLAWYQQKPGQPPKLLIYTASNLAPGVSSRFKGSRSGTESTLIISDLECADAATYYCQCTYYGTSYMELGAFGGGTEVVVK

SEQ ID NO: 258 - protein, 116 aa, Artificial Sequence
  Monoclonal antibody segment
QSVKESGGRLVTPGTPLTLTCTVSGFSINDFHMCWVRQAPGKGLEYIGMIGTGGSAYYATWTKGRFTISRTSTTVDLKITSPTTEDTATYFCARRDWSDGGYSLLWGQGTLVTVSS

SEQ ID NO: 259 - protein, 110 aa, Artificial Sequence
  Monoclonal antibody segment
ELDLTQTPASVSEPVGGTVTIKCQASQSIRTYLNWYQQKPGQPPKLLIYSASTLASGIPSRFKGSGSGTQFTLTISDLECADAATYYCQQGYYWSDVENTFGGGTEVEIN

SEQ ID NO: 260 - protein, 111 aa, Artificial Sequence
  Monoclonal antibody segment
QSVKESGGRLVTPGTPLTLTCTVSGFSLSINAINWVRQAPGKGLEWIGIITNIGNTYYASWAKGRFTISRTSTTVDLTMTSLTTEDTATYFCARNSYFDPWGPGTLVTISS

SEQ ID NO: 261 - protein, 110 aa, Artificial Sequence
  Monoclonal antibody segment
ELVLTQTPSPVSAAVGGTVSISCQASQSVDSNNYCSWYQQKPGQPPKLLIHKASKLASGVPSRFKGSGSGTQFTLTINDLECDDAATYYCAGGLGDNIYAFGGGTEVEIN

SEQ ID NO: 262 - protein, 115 aa, Artificial Sequence
  Monoclonal antibody segment
QSVKESGGRLVTPGTPLTLTCTVSTIDVSKYAVIWVRQAPGKGLEYIGIISSSGSTYYASWAKGRFTISKTSSTTVDLKMTSPTAEDTATYFCARGGNGVAFKLWGQGTLVTISS

SEQ ID NO: 263 - protein, 110 aa, Artificial Sequence
  Monoclonal antibody segment
ELDLTQTPASVEAAVGGTVTINCQASQSIYSSLAWYQQKPGQPPKLLIYETSKVESGVPSRFSGSGSGTQFTLTISDLECADAATYYCQSHDYISSSGMPFGGGTELEIL

SEQ ID NO: 264 - DNA, 381 bp, Artificial Sequence
  Monoclonal antibody segment
acaggtgtccactcggaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgggcggaaggattgatgcttttgatatctggggccaagggaccacggtcagcgtctcctcagcatccaccaagggc

SEQ ID NO: 265 - DNA, 351 bp, Artificial Sequence
  Monoclonal antibody segment
acagacgctcgctgcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggactaaagtggatatcaaacgtacggtggctgca

SEQ ID NO: 266 - DNA, 390 bp, Artificial Sequence
  Monoclonal antibody segment
  misc_feature (275)..(275): n is a, c, g, or t
acaggtgtccactcggaagtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctganatctgacgacacggccgtgtattactgtgcgagaaggtatagcagcagcttacgcgcttttgatatctggggccaagggacaatggtcagcgtctcctcagcatccaccaagggc

SEQ ID NO: 267 - DNA, 351 bp, Artificial Sequence
  Monoclonal antibody segment
acagacgctcgctgcgacatccagatgacccagtctccatcctccctgtctgcatctgtaggagacagagtctccatcacttgccgggcaagtcagagcattagtagatatttaaattggtatcagcagaaaccagggaaagcccctaagctcctgatctatgctgcatccagtttgcaaagtggggtcccatcaaggttcagtggcagtggatctgggacagatttcactctcaccatcagtagtctgcaacctgaagattttgcaacttattactgtcaacagagttacaggaccccctacacttttagccaggggaccaagctggagatcaaacgtacggtggctgca

SEQ ID NO: 268 - DNA, 387 bp, Artificial Sequence
  Monoclonal antibody segment
acaggtgtccactcggaagtgcagctggtgcagtctggaggaggcttggtccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagacacaattccaagaacacgctgtatcttcaaatgaacagcctgagagctgaggacacggccgtgtattactgtgcgagagaatcggcagtggccgggtggggtatggacgtctggggcaaagggaccacggtcaccgtctcttcagcatccaccaagggc

SEQ ID NO: 269 - DNA, 351 bp, Artificial Sequence
  Monoclonal antibody segment
acagacgctcgctgcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcgggacgttcggccaagggaccaagctggaaatcaaacgtacggtggctgca

SEQ ID NO: 270 - DNA, 387 bp, Artificial Sequence
  Monoclonal antibody segment
acaggtgtccactcggaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaggtctcgcagcatcgactcggttcgacccctggggccagggcaccctggtcaccgtctcctcagcatccaccaagggc

SEQ ID NO: 271 - DNA, 351 bp, Artificial Sequence
  Monoclonal antibody segment
acagacgctcgctgcgacgtccagatgacccagtctccatcctccctgtctgcatctgtcggagacagagtcaccatcacttgccggccaagtcagagcattaacaactatttaaattggtatcagcagaaaccaggggaagcccctaagctcctgatctatgctgcatccaatttgcaaagtggggtcccatcaaggttcagtggcagtggatccgggacagatttcactctcaccatcagcagtctgcaacctgaagattttggaacttactactgtcaacagagttacagtatccctctcactttcggcggagggactacagtggaaatcaaacgtacggtggctgca

SEQ ID NO: 272 - DNA, 378 bp, Artificial Sequence
  Monoclonal antibody segment
acaggtgtccactcggagcagtcggtggaggagtccgggggtcgcctggtcacgccggggacacccctgacactcacctgcacagtctctggaatcgacctcagtaattatgcaataaactgggtccgccaggctccagggaaggggctggaatggatcggaatgattagtagtggtgatgtcacatactacgcgagctgggcgaaaggccgattcatcatctccagaacctcgtcgaccacggtggatctgaaaatgaccagtccgacaatcgaggacacggccacctatttctgtgccagatttttcgctggtaggagtgaatccttgtggggccaaggcaccctggtcaccgtctcttcagggcaacctaaggct

SEQ ID NO: 273 - DNA, 367 bp, Artificial Sequence
  Monoclonal antibody segment
acagatgccagatgcgagctcgatctgacccagacaccatcctccgtgtctgccgctgtgggaggcacagtcaccatcaactgccagtccagtcagagtgttaataagaacaactacttatcctggtatcaacaaaaaccagggcagcctcccaagctcctaatctattctgcatccgctctggcatctggggtctcatcgcggttcagcggcagtggatctgggacagagttcactctcaccatcagcgacgtgcagtgtgacgatgctgccacttactactgtcaaggcacttattatgatagtggttggtacaatgctttcggcggagggaccgaggtggaaatcaaaggtgatccagttgcac

SEQ ID NO: 274 - DNA, 378 bp, Artificial Sequence
  Monoclonal antibody segment
acaggtgtccactcgcagtcgttgaaggagtccgggggtcgcctggtcaagcctgacgaaaccctgacaatcacctgcacagcctctggaatcgacctcactagcggtacagtgggctggttccgccaggctccagggaagggactagaatggatcggaattattcgtggtagtggtagcacatattacgcgacctgggcgaaaggccgattcaccatctccaaaacctcgaccacggtggatctgaagctcaccagtccgacaaccgaggacacggccacctatctctgtgccagagccttccctggttataatactttctttaacttctggggccaaggcaccctggtcaccatctcttcagggcaacctaaggct

SEQ ID NO: 275 - DNA, 364 bp, Artificial Sequence
  Monoclonal antibody segment
acagatgccagatgcgagctcgatctgacccagacaccatcctccgtggaggcagctgtgggaggcacagtcaccatcaagtgtcaggccagtgagagcattggcaatgcattagcctggtatcagcagaaaccagggcagcctcccaaactcctgatctggaaggcatccaatctagaatctggggtccccccgcggttcaaaggcagtggatctgggacagagttcactctcaccattagcgacctggaatgtgccgatgctgccacttactattgtcaaaggtattatttcaatggtgttagtgttgagatagttttcggcggagggaccgaggtggtggtcaaaggtgatccagttgcac

SEQ ID NO: 276 - DNA, 390 bp, Artificial Sequence
  Monoclonal antibody segment
acaggtgtccactcggagcagtcggtggaggagtccgggggtcgcctggtcacgcctgggacacccctgacactcacctgcacagcctctggattctccctcagtagctactacatgacctgggtccgccaggctccagggaaggggctggaatacatcggaatcattagtgatcgtggtagcacatactacgcgagctgggcgaaaggccgattcaccatcgccaaaacctcgtcgaccacggtggatctgaaactgaccagtctgacaaccgaggacacggccacctatttctgttatagaggtgttgatggtagtggtgggtatgctgccggtgacttgtggggccaaggcaccctggtcaccatctcttcagggcaacctaaggct

SEQ ID NO: 277 - DNA, 367 bp, Artificial Sequence
  Monoclonal antibody segment
acagatgccagatgcgagctcgtgctgacccagacaccagcctccgtggaggcagctgtgggaggcacagtcaccatcaagtgtcaggccagtgaggacatttatagctacttcgcctggtatcagcagaaaccagggcagcgtcccaggctcctgatctatgaagcatccaaagtggcatctggggtcccatcgcggttcaaaggcggtggatctgggacacaattcactctcaccatcagcgacctggagtgtgccgatgctgccacttactactgtcaaaacaatttttatatgattggtgatgattatggtggtactttcggcggagggaccgaggtggaaatcaaaggtgatccagttgcac

SEQ ID NO: 278 - DNA, 387 bp, Artificial Sequence
  Monoclonal antibody segment
acaggtgtccactcgcagtcggtgaaggagtccgggggtcgcctggtcacgcctgggacacccctgacactcacctgcacagtctctggattctccctcagtagttatgcaatgagctgggtccgccaggctccagggaaggggctggagtggatcggagtcattgatgttactggtaggacatactacgcgagctgggcgaaaggccgattcaccatctccaaaacctcgaccacggtggatctgaaaatcaccagtccgacaaccgaggacacggccacctatttctgtggcagagatagctaccttgactatggtgaccccaatggtcttgatccctggggcccaggcaccctggtcaccatctcctcagggcaacctaaggct

SEQ ID NO: 279 - DNA, 367 bp, Artificial Sequence
  Monoclonal antibody segment
acagatgccagatgcgagctcgtgatgacccagacaccagcctccgtggaggcagctgtgggaggcacagtcaccatcaagtgccaggccagtcagagcattggtagtaatttagcctggtatcagcagaaaccagggcagcctcccaagctcctgatctatactgcatccaatctggcgcctggagtctcatcgcggttcaaaggcagtagatctgggacagagtccactctcatcatcagcgacctggagtgtgccgatgctgccacttactactgtcaatgtacttattatggtactagttatatggaattaggggctttcggcggagggaccgagctggaaatcaaaggtgatccagttgcac

SEQ ID NO: 280 - DNA, 375 bp, Artificial Sequence
  Monoclonal antibody segment
acaggtgtccactcgcagtcgttggaggagtccgggggtcgcctggtcacgcctgggacacccctgacactcacctgcacagtctctacaatcgacgtcagtaagtatgcagtgatctgggtccgccaggctccagggaaggggctggaatacatcggaatcattagtagtagtggtagcacatactacgcgagctgggcgaaaggccgattcaccatctccaaaacctcatcgaccacggtggatctgaaaatgaccagtccgacagccgaggacacggccacctatttctgtgccagagggggtaatggtgtcgcctttaaattatggggccaaggcaccctggtcaccatctcttcagggcaacctaaggct

SEQ ID NO: 281 - DNA, 361 bp, Artificial Sequence
  Monoclonal antibody segment
acagatgccagatgcgagctcgatatgacccagacaccagcctccgtggaggcagctgtgggaggcacagtcaccatcaattgccaggccagtgagagcattaacgatagattagcctggtatcagctgaaaccagggcagcctcccaaactcctgatctacgaaacatcccaagtggaatctggggtcccatcgcggttcagcggcagtggatctgggacacagttcactctcaccatcagcgacctggagtgtgccgatgctgccacttactactgtcaaagccatgattatattagtagtagtggtatgcctttcggcggagggaccgaggtggtggtcaaaggtgatccagttgcac

SEQ ID NO: 282 - DNA, 390 bp, Artificial Sequence
  Monoclonal antibody segment
acaggtgtccactcgcagtcggtgaaggagtccgggggtcgcctggtcacgcctgggacacccctgacactcacctgcacaatgtctggattctccctcggtagggatgcgctgacctgggtccgccaggctccaggaaaggggctacaatggatcgcattcatcaatgatgatggtgacactgactatccaaactgggcgagaggccgattcaccatctccaaaacctcgtcgaccacggtggatctgaaaatcaccagtcctacaaccgaggacacggccacctatttctgtgccagagtgggggctgaatatcttggttggggtaatggttttaatttgtggggccaaggcaccctggtcaccatctcctcagggcaacctaaggct

SEQ ID NO: 283 - DNA, 370 bp, Artificial Sequence
  Monoclonal antibody segment
acagatgccagatgcgagctcgatatgacccagacaccagcctccgtgtctgcagctgtgggaggcacagtcaccatcaactgccaggccagtcagagtctttataataagaaaaatttagcctggtatcagcagaaaccagggcagcctcccaagctcctgatctacaaggcttccactctggcatctggggtcccatcgcggttcgaaggcagtggatctgggacagagtacgctctcaccatcagcggcttgcagtgtgacgatgctgccacttactactgtcaaggcgaatttagttgtgttgctgctgattgtttttctttcggcggagggaccgaggtggtggtcaaaggtgatccagttgcac

SEQ ID NO: 284 - DNA, 390 bp, Artificial Sequence
  Monoclonal antibody segment
acaggtgtccactcgcagtcggtgaaggagtccgggggagacctggtcaagcctggggcatccctgacactcacctgtacagcctctggattctccttcagtagcagccagtacatgtgctgggtccgccaggctccagggaaggggctggagtggatcgcatgcatttatggtggtagtagtggtctcacaaactacgcgacctgggcgaaaggccgattcaccgtctccaaaacctcgtcgaccacggtgactctgcaaatgaccagtctgacagacgcagacacggccacgtatttctgtgcgagagctattggtcctacttattatgcttttgatccctggggcccaggcaccctggtcaccgtctcttcagggcaacctaaggct

SEQ ID NO: 285 - DNA, 361 bp, Artificial Sequence
  Monoclonal antibody segment
acagatgccagatgcgagctcgatctgacccagactccatcctccgtgtctgcagctgtgggagacacagtcaccatcaagtgccaggccagtgagagcatttacagttacttagcctggtatcagcagaaaccagggcagcctcccaagctcctgatctattatacatccactctggcatctggggtcccatcgcggttcaaaggcagtggatctgggacagagttcactctcaccatcagcgacctggagtgtgccgatgctgccacttactactgtcaaaacacttatggtagtagtagtaatgcttttccttttggcggagggaccgaggtggaaatcaaaggtgatccagttgcac

SEQ ID NO: 286 - DNA, 369 bp, Artificial Sequence
  Monoclonal antibody segment
acaggtgtccactcgcagtcggtgaaggagtccgggggtcgcctggtcacgcctgggacacccctgacactcacctgcacagtctctggattctacctcagtacccatggaatgacctgggtccgccaggctccagggaaggggctggaatacatcggagtcattgttactagtggtaacacatactacgcgacctgggcgactggccgattcaccatctccaaaacctcgaccacggtggatctgaaaatcaccagtccgacaaccgaggacacggccacctatttctgtgccagacctggttataaaagtgtccccttgtggggccaaggcaccctggtcaccatctcttcagggcaacctaaggct

SEQ ID NO: 287 - DNA, 367 bp, Artificial Sequence
  Monoclonal antibody segment
acagatgccagatgcgagctcgatctgacccagacaccagcctccgtgtctgcagctgtgggaggcacagtcaccatcaattgccaggccagtgaggacatttacagtggtttgacctggtatcagcagaaaccagggcagcctcccaagctcctgatctacagggcatccactctggcatctggggtcccaccgcggttcaaaggcagtggatctgggacagaattcactctcaccatcagcgacctggagtgtgacgatgctgccacttactactgtcaaaacaattatggtagtagtagtaataattatgtttttgctttcggcggagggaccgaggtggaaatcaaaggtgatccagttgcac

SEQ ID NO: 288 - DNA, 387 bp, Artificial Sequence
  Monoclonal antibody segment
acaggtgtccactcgcagtcggtgaaggagtccgggggtcgcctggtcacgcctgggacacccctgacactcacctgtacactctctggaatcgacctcagtagctatgcaatgggctgggtccgccaggctccagggaaggggctggaatacatcggaatcattggtgctagtggtaccacttactacgcgaactgggcgaaaggccgattcaccatctccagaacctcgaccacggtggatctgaaaatcaccagtccgacaaccgaggacacggccacctatttctgtgccagagctggttatgctggtactggttatcctactcgtttggatctctggggccagggcaccctggtcaccatctcttcagggcaacctaaggct

SEQ ID NO: 289 - DNA, 367 bp, Artificial Sequence
  Monoclonal antibody segment
acagatgccagatgcgagctcgatatgacccagacaccagcctccgtggaggcagctgtgggaggcacagtcaccatcaagtgccaggccagtcagagtattagtagtagctacttagcctggtatcagcagaaaccagggcagcctcccaagctcctgatctacaaggcatccactctggaatctggggtcccatcgcggttcagcggcagtggatctgggacagagttcactctcaccatcagcggcgtgcagtgtgacgatgctgccacttactactgtctaggcggttattttagttttagtaattatgattatggtttcggcggagggaccgaggtggaaatcaaaggtgatccagttgcac

SEQ ID NO: 290 - DNA, 393 bp, Artificial Sequence
  Monoclonal antibody segment
acaggtgtccactcgcagtcggtgaaggagtccgggggagacctggtcaagcctggggcatccctgacactcacctgcacagcctctggattcgacttcagtagcaatacaatgtgctgggtccgccaggctccagggaaagggctggaatatattggatacattagttatagtggtgctacgcattacgcgagctgggcgaaaggccgattcaccatctccaaaaccttgtcgaccacggtgactctgcaaatgaccagtctgacagacgcggacacggccacctatttctgtgcgagagatcggggttatgttgatggtagttattatggtgactttaacttgtggggcccaggcaccctggtcaccgtctcttcagggcaacctaaggct

SEQ ID NO: 291 - DNA, 358 bp, Artificial Sequence
  Monoclonal antibody segment
acagatgccagatgcgagctcgtgatgacccagactccatcttccgcgtctgaacctgtgggaggcacagtcaccatcaagtgccaggccagtcagagcattgctagtagattagcctggtttcagcagaaaccagggcagcctcccaagctcctaatatacagggcatccactctggaatctggggtctcatcgcggttcaaaggcagtggatttgggacagagttcactctcaccatcagcgacctggagtgtgccgatgctgccacttactactgtcaaagctattatggtagtggttatggcaagactttcggcggagggaccgacgtggtggtcaaaggtgatccagttgcac

SEQ ID NO: 292 - DNA, 381 bp, Artificial Sequence
  Monoclonal antibody segment
acaggtgtccactcgcagtcgctggaggagtccgggggtcgcctggtcacgcctgggacacccctgacactcacctgcacagtctctggattctccctcagtagctatccaatgagctgggtccgccaggctccagggaaggggctggaatggatcggaatcattactagtggtggtagtatattttacgcgacctgggcgaatggtcgattcaccatctccaaaacctcgaccacggtggatctgaaaatcaccagtccgacaaccgaggacacggccacctatttctgtgccagaggttatagtagtggtactatgatgtcttctaacttgtggggccaaggcaccctggtcaccgtctcctcagggcaacctaaggct

SEQ ID NO: 293 - DNA, 361 bp, Artificial Sequence
  Monoclonal antibody segment
acagatgccagatgcgagctcgtgctgacccagacaccagcctccgtgtctgaacctgtgggaggcacagtcaccatcaagtgccaggccagtgagagcattagtacttacttatcctggtatcagcagaaaccagggcagcgtcccaagctcctgatctacagggcatccactctggaatctggggtcccatcgcggttcaaaggcagtggatctgggacagagtacactctcaccattagcggcgtgcagtgtgccgatgctgccacttactactgtcaacagggttatacttataaaaatgttgataatgctttcggcggagggaccgaggtggtggtcaaaggtgatccagttgcac

SEQ ID NO: 294 - DNA, 381 bp, Artificial Sequence
  Monoclonal antibody segment
acaggtgtccactcggagcagtcgttggaggagtccgggggtcgtctggtcatgcctggaggatccctgacactcacctgcacagtctctggaatcgacctcagtagcaatacaatgggctggttccgccaggctccagggaaggggctggaatggatcgggattatgcgtggtggtggtagcacatactacgcgacctgggcgaaaggccgattcaccatctccaaaacctcgaccacggtggatctgaaactcaccagtccgacaaccgaggacacggccacctatttctgtgccagagccttccctggttataattctttctttaacttctggggccaaggcaccctggtcaccatctcttcagggcaacctaaggct

SEQ ID NO: 295 - DNA, 352 bp, Artificial Sequence
  Monoclonal antibody segment
acagatgccagatgcacccagacaccagcctccgtgtctgaacctgtgggaggcacagtcaccatcaagtgccaggccagtcacaacattaatagtgctttggcctggtatcagcagaaaccagggcagcctcccaagctcctgatctacaaggcatccactctggcatctggggtcccaccgcggttcaaaggcagtggatctgggacagagttcactctcaccatcagcgacctggagtgtgccgatgctgccacttactactgtcaaaggcattatttcgatggtgccagtgctgatatagctttcggcggagggaccgaggtggaaatcaaaggtgatccagttgcac

SEQ ID NO: 296 - DNA, 399 bp, Artificial Sequence
  Monoclonal antibody segment
  misc_feature (25)..(25): n is a, c, g, or t
acaggtgtccactcgcagtcggtgnaggagtccgggggaggcctggtccagcctgagggatccctgacactcacctgcacagcctctggattctccttcagtggcggctacaacatgtgctgggtccgccaggctccagggaaggggctggagtggatcgcatgcatttatgctggtagtagtggtagcacttactacgcgagctgggcgaaaggccgattcaccatctccaaaacctcgtcaaccacggtgactctgcaaatgaccagtctgacagccgcggacacggccacctatttctgtgcgagaggggcttatgctggtggtagttaccccccctactttaacttgtggggcccaggcaccctggtcaccatctcctcagggcaacctaaggtt

SEQ ID NO: 297 - DNA, 364 bp, Artificial Sequence
  Monoclonal antibody segment
  misc_feature (20)..(20): n is a, c, g, or t
  misc_feature (24)..(26): n is a, c, g, or t
acagatgccagatgcgagcncctnnngacccagacaccagcctccgtgtctgagcctgtgggaggcacagtcaccatcaagtgccaggccagtcagagcattagaagttacttagcctggtatcagcagaaagcagggcagcctcccaagctcctgatctacagggcatccactctggcatctggggtcccatcgcggttcaaaggcagtggatctgggacagagtacactctcaccatcagcgacctggagtgtgccgatgctgccacttactactgccaaaatgtttattatcttagtagtagtacatatggtgctttcggcggagggaccgaggtggtggtcaaaggtgatccagttgcac

SEQ ID NO: 298 - DNA, 376 bp, Artificial Sequence
  Monoclonal antibody segment
acaggtgtccactcgcgagcagtcggtgaaggagtccgggggtcgcctggtcacgcctgggacacccctgacactcacctgcacagtctctggattctccctcagtaactatgcaatgagctgggtccgccaggctccagggaaggggctggaatggatcggaatcattgttactagtggtagcacatactacgcgagctgggcaaaaggccgattcaccatctccaaaacctcgaccacggtggatctgaaaatgaccagtctgacagccgcggacacggccacctatttctgtgccagacatgcgtatagtattggttcgagcttgtggggccaaggcaccctggtcaccatctcttcagggcaacctaaggct

SEQ ID NO: 299 - DNA, 361 bp, Artificial Sequence
  Monoclonal antibody segment
acagatgccagatgcgagctcgatctgacccagacaccaccttctgcgtctgaacctgtgggaggcacagtcaccatcaagtgccaggccagtgagagcatcagtgttagattagcctggtatcagcagaaaccagggcagcctcccaagctcctgatctacaaggcatccaatctgccatctggggtcccatcgcggttcaaaggcagtggatctgggacagagttcactctcaccatcagcgacctggagtgtgacgatgctgccacttattactgtcgcaactattatgatattagtaataatggtgctggttttggcggagggaccgaggtggaaatcaaaggtgatccagttgcac

SEQ ID NO: 300 - DNA, 384 bp, Artificial Sequence
  Monoclonal antibody segment
acaggtgtccactcggagcagtcggtgaaggagtccgggggaggcctcttcaagccaacggataccctgacactcacctgcacagtctctggaatcgacctcagtagctatgcaatgacctgggtccgccaggctccaggggaggggctggagtggatcggaaccattagtagtagtggtagcacatactacgcgagctgggcgaaaggccgattcaccatctccaaaacctcgtcgaccacggtggatctgaaaatgaccagtctgacagccgcggacacggccacctatttctgtgccagaggatctggtggtgctgttacttattatggcgtgtggggccaaggcaccctggtcaccgtctcttcagggcaacctaaggct

SEQ ID NO: 301 - DNA, 361 bp, Artificial Sequence
  Monoclonal antibody segment
acagatgccagatgcgagctcgatctgacccagacaccagcctccgtgtctgaacctgtgggaggcacagtcaccatcaagtgccaggccagtcagagcattggtagtagcttagcctggtatcagcagaaaccagggcagcctcccaagctcctgatctacagggcatccactctggcatctggggtcccatcgcggttcagtggcagtggatctgggacagagttcactctcaccatcagcgacctggagtgtgcccatgctgccacttactactgtcaaaactatattagtagtagtacttttgggaatgatttcggcggaggaaccgaggtggtggtcaaaggtgatccagttgcac

SEQ ID NO: 302 - DNA, 372 bp, Artificial Sequence
  Monoclonal antibody segment
acaggtgtccactcgcagtcggtggaggagtccgggggtcgcctggtcacgcctgggacacccctgacactcacctgcacagtctctggaatcgacctcagtagctatgcaatcaactgggtccgccaggctccaggggaggggctggaatggatcggaatgattagtagtagtgataacacatactacgcgaactgggcgaaaggccgattcaccatctccagagactcgaccacggtggatctgaaaatcaccagtccgacaaccgaggacacggccacctatttctgtgccagattttacgctggtagaagttattccttgtggggccaaggcaccctggtcaccatctcctcagggcaacctaaggct

SEQ ID NO: 303 - DNA, 366 bp, Artificial Sequence
  Monoclonal antibody segment
acagatgccagatgcgagctcgatctgacccagacaccagcctccgtgtctgcagctgtgggaggcacagtcaccatcagttgccagtccagtgagagtgtttataagagcaactatttatcttggtatcagcaaaaagcagggcagcctcccaagctcctaatctatgctgcagccactctggcatctggggtcccatcgcggttcaaaggcagtggatctgggacacagttcactctcaccatcagcggcgtgcagtgtgacgatgctgccacttactactgtcaaggcacttattatagtagtggttggtacaatgctttcggcggagggaccgaggtggtggtcaaaggtgatccagttgca

SEQ ID NO: 304 - DNA, 387 bp, Artificial Sequence
  Monoclonal antibody segment
acaggtgtccactcggagcagtcggtggaggagtccgggggtcgcctggtcacgcctgggacacccctgacactcacctgcacagcctctggattctccctcagtacttactacatgagttgggtccgccaggctccagggaaggggctggaatggatcgggatcattcgtggtggtggtgaacctgtatggtacgcggactgggcaaaaggccgattcaccatctccagaacctcgaccacggtggatctgaaaatcaccagtccgacgcccgaggacacggccacctatttctgtgccagaaggctggatggttttgttagggcttatctggacttgtggggccagggcaccctggtcaccgtctcttcagggcaacctaaggct

SEQ ID NO: 305 - DNA, 358 bp, Artificial Sequence
  Monoclonal antibody segment
acagatgccagatgcacccagacaccagcctccgtggaggcagctgtgggaggcacagtcaccatcaaatgccaggccagtgagagtattagtagttggttggcctggtatcagcagaaaccagggcagcctcccaagctcctgatctatgaagcattcagtctggcctctggggtcccatcgcggttcagcggcagtggatctgggacagagttcactctcaccatcagcgacctggagtgtgacgatgctgccatttattactgtcaaagtgcttattatggtggtacttatgttggaggtttcggcggagggaccgaggtggtggtcaaaggtgatccagttgcatccagttgca

SEQ ID NO: 306 - DNA, 381 bp, Artificial Sequence
  Monoclonal antibody segment
acaggtgtccactcggagcagtcggtggaggagtccgggggtcgtctggtcatgcctggaggatccctgacactcacctgcacagtctctggaatcgacctcagtagcaatacaatgggctggttccgccaggctccagggaaggggctggaatggatcgggattatgcgtggtggtggtagcacatactacgcgacctgggcgaaaggccgattcaccatctccaaaacctcgaccacggtggatctgaaactcaccagtccgacaaccgaggacacggccacctatttctgtgccagagccttccctggttataattctttctttaacttctggggccaaggcaccctggtcaccatctcttcagggcaacctaaggct

SEQ ID NO: 307 - DNA, 363 bp, Artificial Sequence
  Monoclonal antibody segment
acagatgccagatgcgagctcgatctgacccagacaccagcctccgtgtctgaacctgtgggaggcacagtcaccatcaagtgccaggccagtcacaacattaatagtgctttggcctggtatcagcagaaaccagggcagcctcccaagctcctgatctacaaggcatccactctggcatctggggtcccaccgcggttcaaaggcagtggatctgggacagagttcactctcaccatcagcgacctggagtgtgccgatgctgccacttactactgtcaaaggcattatttcgatggtgccagtgctgatatagctttcggcggagggaccgaggtggtggtcaaaggtgatccagttgca

SEQ ID NO: 308 - DNA, 375 bp, Artificial Sequence
  Monoclonal antibody segment
acaggtgtccactcggcagcggtgaaggagtccgggggtcgcctggtcacgcctgggacacccctgacactcacctgcacagtctctggattctccctcagtaactatgcaatggggtgggtccgccagcctccagggaaggggctggagtggatcggattcagtggttatcctggtcgcacatactatgcgagttgggcgaaaggccgattcaccatctccaaaacctcgaccacggtggatctgaaaatcaccagtccgacagccgaggacacggccacctatttctgcgccagacattatggtagcactaacgggcttaacttgtggggccaaggcaccctggtcaccgtctcttcagggcaacctaaggct

SEQ ID NO: 309 - DNA, 360 bp, Artificial Sequence
  Monoclonal antibody segment
acagatgccagatgcgagctcgatctgacccagacaccagcctccgtgtctgaacctgtgggaggcacagtcaccatcaagtgccaggccagtcagaacaataataactacttagcctggtatcaacagaaaccagggcagcctcccaagctcctgatctatgctgcatccagtctggcatctggggtcccatcgcggttcaaaggcagtggatctgggacacagttcactctcaccatcagcgacctggagtgtgacgatgctgccacttactactgtcaatgtactgattatggtcctacttatcttggtactttcggcggagggaccgaggtggaaatcaaaggtgatccagttgca

SEQ ID NO: 310 - DNA, 396 bp, Artificial Sequence
  Monoclonal antibody segment
acaggtgtccactcgcagtcgttggaggagtccgggggagacctggtcaagcctggggcatccctgacactcacctgcacagcctctggattctccttcagtagctactacgacatgtgctgggtccgccaggctccagggaaggggctggagtggatcgcatgtatttatattggtaatagtggtaacacttactacgcgagctgggcgaaaggccgattcaccatctccagaacctcgtcgaccacggtggatctgaaaatgtccagtctgacagcggcggacacggccagttatttctgtgcgagaggtgctagtggttatagtggttatggttggtttattttgtggggcccaggcactctggtcaccatctcttcagggcaacctaaggct

SEQ ID NO: 311 - DNA, 360 bp, Artificial Sequence
  Monoclonal antibody segment
acagatgccagatgcgagctcgtgatgacccagactccagcctccgtgtctgcagctgtgggaggcacagtcaccatcaagtgccaggccagtcagagcattggtagtaatttggcctggtatcagcagaaaccagggcagcgtcccaaactcctgatctatgatgcatctgatctggcatctggggtcccatcgcggttcaaaggcagtggttctgggccagagtacaccctcaccatcagcggcgtggagtgtgacgatgctgccacttactactgtcaacagggtgatacttatattaatgttgataatattttcggcggagggaccgaggtggaaatcaaaggtgatccagttgca

SEQ ID NO: 312 - DNA, 381 bp, Artificial Sequence
  Monoclonal antibody segment
acaggtgtccactcggagcagtcggtggaggagtccgggggtcgcctggtaacgcctgggacacccctgacactcacctgcacagtctctggaattgacctcagtaactatataatgggctgggtccgccaggctccagggaaggggctggaatggatcggaatcattgctagtaatggtggcacagtctacgcgaactgggcgaaaggccgattcaccatctccaaaacctcgaccacggtggatctgaaaatgaccagtccgacacccgaggacacggccacctatttctgtgccagaggtgctggtactacttggtcctcctttaacttgtggggccaaggcaccctggtcaccatctcttcagggcaacctaaggct

SEQ ID NO: 313 - DNA, 350 bp, Artificial Sequence
  Monoclonal antibody segment
acagatgccagatgcgagctcgtgctgacccagacaccagcctctgtgtctgaacctgtgggcggcacagtcaccatcaattgccaggtcagtcagagcattgataactacttgaactggtatcaccagaaaccagggcagcctcccaagcgcctgatctacagggcatccactctggcatctggggtcccatcgcggttcagaggcagtggatctgggacacagttcactctcaccatcagcgacctggagtgtgccgatgctgccacttactactgtcaaagctattatggtagtagtactaatgctttcggcggagggaccgaggtggtggtcaaaggtgatccagt

SEQ ID NO: 314 - DNA, 372 bp, Artificial Sequence
  Monoclonal antibody segment
acaggtgtccactcggagcagtcggtggaggagtccgggggtcgcctggtcacgcctgggacacccctgacactcacctgcacagtctctggattctccctcagtagttatgcaatgaactgggtccgccaggctccagggaagggactggaatggatcggactcatttatgctagtggtaccactcactacgcgagctgggtgaatggccgattcaccatctccaaaacctcgaccacggtgactctgaaaatgaccagtctgacagccgcggacacggccacctatttctgtgccagagatagttatagtactggtaacttgtggggccaaggcaccctggtcaccatctcttcagggcaacctaaggct

SEQ ID NO: 315 - DNA, 351 bp, Artificial Sequence
  Monoclonal antibody segment
acagatgccagatgcgagctcgtgctgacccagacaccagcctccgtgtctgaacctgtgggaggcacagtcaccatcaagtgccaggccagtcagagcatttacagcagtttagtctggtatcagcagaaaccagggcagcctcccaagctcctgatctatggtgcgtccaatctggaatctggggtcccatcgcggttcaaaggcagtggatctgggacagagtacactctcaccatcagcggcgtgcagtgtgccgatgctgccacttactactgtcaatgtagtggttataatagtcgtgttttcggcggagggaccgaggtggaaatcaaaggtgatccagttgca

SEQ ID NO: 316 - DNA, 387 bp, Artificial Sequence
  Monoclonal antibody segment
acaggtgtccactcgagcagttcggtggaggagtccgggggaggcctggtcaagcctggggcatccctgacactcacctgcacagcctctggattctccttcagtagcggctactggatgtgctgggtccgccaggctccagggaaggaactggagtggatcgcatgcattggcgctcgtgatagtggtagcgcttactacgcgacctgggcgaaaggccgattcaccatctccaaaatctcgtcgaccacggtgactctgcaaatgaccagtctgacagccgcggacacggccacctatttctgtgcgagatctggtgctagtttccactttaaattgtggggcccaggcaccctggtcaccatctcttcagggcaacctaaggct

SEQ ID NO: 317 - DNA, 351 bp, Artificial Sequence
  Monoclonal antibody segment
acagatgccagatgcgagctcgtgctgacccagacaccagcctccgtgtctgaacctgtgggaggcacagtcaccatcaagtgccaggccagtcagaacatcggtagctacttagcctggtatcagcagaaaccagggcagcctcccaagctcctgatctatggtgcgtccaatctggaatctggggtcccatcgcggttcaaaggcagtggatctgggacagagtacactctcaccatcagcggcgtgcagtgtgccgatgctgccacttactactgtcaatgtagtggttataatagtcgtgttttcggcggagggaccgaggtggtggtcaaaggtgatccagttgca

SEQ ID NO: 318 - DNA, 387 bp, Artificial Sequence
  Monoclonal antibody segment
acaggtgtccactcgcagtcgctggaggagtccgggggagacctggtcacgcctgggacacccctgacactcacctgcacagtctctggattctccctcagtacctacaacattcaatgggtccgccaggctccagggaagggtctggaatgcatcggattcattaatggtggtggtagcgcatactacgcgagctgggtgaatggtcgattcaccgtctccaaagcctcgaccacggtgactctgaaaatcaccgctccgacaaccgaggacacggccacctatttctgtgccagaggttattatgctggtgctgattggaatatgccgttggatctctggggccagggcacgctggtcaccgtctcttcagggcaacctaaggct

SEQ ID NO: 319 - DNA, 360 bp, Artificial Sequence
  Monoclonal antibody segment
acagatgccagatgcgagctcgtgctgacccagacaccttcgcccgtgtctgcaggtgtgggaggcacagtcaccatcaagtgccagtccagtgagagcattggcactgcattagcctggtatcagcagaaactagggcagcctcccaagctcctgatctatgctgcagcccttctggcatctggggtctcatcgcggttcaaaggcagcggatctgggacagagtacactctcaccatcagcgacctggagtgtgacgatgctgccacttactactgtcaatttacttattatggtaatagttatgggaatgctttcggcggagggaccgaggtggaaatcaaaggtgatccagttgca

SEQ ID NO: 320 - DNA, 399 bp, Artificial Sequence
  Monoclonal antibody segment
acaggtgtccactcgcagtcggtgaaggagtccgggggagacctggtcaagcctgagggatccctgacactcacctgcacagcctctggattctccttcagtggcggctacgacatgtgctgggtccgccaggctccagggaaggggctggagtggatcgcatgcatttatgctggtagtagtggtagcacttactacgcgagctgggcgaaaggccgattcaccatctccaaaacctcgtcgaccacggtgactctgcaaatgaccagtctgacagccgcggacacggccacctatttctgtgcgagaggggcttatgctggtggtggttaccccccctactttaatttctggggcccaggcaccctggtcaccatctcttcagggcaacctaaggct

SEQ ID NO: 321 - DNA, 360 bp, Artificial Sequence
  Monoclonal antibody segment
acagatgccagatgcgagctcgtgatgacccagacaccagcctccgtggaggcagctgtgggaggcacagtcaccatcaattgccaggccagtgagagcattaacgatagattagcctggtatcagctgaaaccagggcagcctcccaaactcctgatctacgaaacatccaaagtggaatctggggtcccatcgcggttcagcggcagtggatctgggacacagttcactctcaccatcagcgacctggagtgtgccgatgctgccacttactactgtcaaagccatgattatattagtagtagtggtatgcctttcggcggagggaccgagctggaaatcaaaggtgatccagttgca

SEQ ID NO: 322 - DNA, 375 bp, Artificial Sequence
  Monoclonal antibody segment
acaggtgtccactcggagcagtcggtgaaggagtccgggggtcgcctggtcacgcctgggacacccctaacactcacctgcacagtctctggattctccctcagtagcagtggaatgagctgggtccgccaggctccagggaaggggctggaatggatcggactcacttttcctagtggtagcgcatcctacgcgagctgggcgaaaggccgatttaccgtctccaggacctcgaccacggtggatctgaatctcaccagtccgacaaccgaagacacggccacctatttctgtgcccatgttggtgctactactggtcgtgacttctggggccaaggcaccctggtcaccgtctcctcagggcaacctaaggct

SEQ ID NO: 323 - DNA, 357 bp, Artificial Sequence
  Monoclonal antibody segment
acagatgccagatgcgagctcgtgatgacccagactgcatcgtccgtgtctgcagctgtgggaggcacagtcaccatcagttgccagtccagtcagaatatttataacaaccgctggtcctggtatcagcagaaaccagggcagcctcccaagctcctgatctaccaggcatccactctggcatctggggtcccatcgcggttcaaaggcagtggatctgggacacagttcactctcaccatcagcggcgtgcagtgtgacgatgctgccacttactactgtgcaggcggttatagtggtacgatttttactttcggcggagggaccgagctggaaatcaaaggtgatccagttgca

SEQ ID NO: 324 - DNA, 372 bp, Artificial Sequence
  Monoclonal antibody segment
acaggtgtccactcggcagcggtgaaggagtccgggggtcgcctggtcacgcctgggacacccctgacactcacctgcacagtctctggaatcgacctcagtgactactacatgacctgggtccgccaggctccagggaaggggctggaatacatcggaatcatttatgctagtggcaacgcatactttgcgagttgggcgaaaggccgattcaccatctccagaacctcgtcgaccacggtggatctgaatgtcgccagtccgacaaccgaggacacggccacctatttttgtgccaggaatattggtggtagtagtaacttgtggggccaaggcaccctggtcaccgtctcttcagggcaacctaaggct

SEQ ID NO: 325 - DNA, 360 bp, Artificial Sequence
  Monoclonal antibody segment
acagatgccagatgcgagctcgatatgacccagacaccagcctccgtgtctgaacctgtgggaggcacagtcaccatcaagtgccaggccagtgagagaatttataataatttagcctggtatcagcagaaaccagggcagcctcccaaactcctcttctattctgcatccactctggcatctggggtcccttcgcgattcaaaggcagtggatctgggacagagttcactctcaccatcagcgacctggagtgtgccgatgcagccatttactactgtcaatgtactggttatggtactacctatgggcttgttttcggcggagggaccgaggtggaaatcaaaggtgatccagttgca

SEQ ID NO: 326 - DNA, 381 bp, Artificial Sequence
  Monoclonal antibody segment
acaggtgtccactcgcagtcggtgaaggagtccgggggaggcctgttccagcctgggggatccctggcactcacctgcaaagcctctggattctccctcagtaaaaattacgtgatgtgctgggtccgccaggctccagggaaggggctggagtggatcgcatgcatttatgctggtgatggtaacacttactacgtgagctgggcgaaaggccgattcaccatctccaaaacctcgtcgaccacggtgactctgcaaatgaccagtctgacagccgcggacacggccacctatttctgtgcgagatgggactatattggtaataataatttgtggggcccaggcaccctggtcaccatctcctcagggcaacctaaggct

SEQ ID NO: 327 - DNA, 360 bp, Artificial Sequence
  Monoclonal antibody segment
acagatgccagatgcgagctcgtgctgacccagacaccagcctccgtggaggcagctgtgggaggcacagtcaccatcaagtgccaggccagtcagaccattagtagctacttagcctggtatcagcagaaaccagggcagcctcccaagctcctgatctacagggcatccactctggcatctggggtcccatcgcggttcaaaggcagtggatctgggacacagttcactctcaccatcagcgacctggagtgtgccgatggtgccacttactactgtcaatgtccttattatggtagtagccatgtgggtgctttcggcggagggaccgaggtggtggtcaaaggtgatccagttgca

SEQ ID NO: 328 - DNA, 369 bp, Artificial Sequence
  Monoclonal antibody segment
acaggtgtccactcgcagtcggtgaaggagtccgggggtcgcctggtcacgcctgggacacccctgacactcacctgcacagtctctggaatcgacctcattagctatccaatgggctgggtccgccaggctccagggaaggggctggaatacatcggaatcattgctactggtggtagcacatactacgcgagctgggcaaaaggccgattcaccatctccacaagctcgaccacggtggatctgaaaatgaccagtctgacagccgcggacacggccacctacttctgtgccagaagatggagtagtgcttatgacttctggggccaaggcaccctggtcaccatctcttcagggcaacctaaggct

SEQ ID NO: 329 - DNA, 360 bp, Artificial Sequence
  Monoclonal antibody segment
acagatgccagatgcgagctcgatctgacccagacaccagcctctgtggaggtagctgtgggaggcacagtcaccatcaagtgccaggccagtcagagcattagtagctacttagcctggtatcagcagaaaccagggcagcctcccaagctcctgatctattatgcatccactctggcatctggggtcccatcgcggttcagcggcagtggatctgggacagagttcactctcaccatcagtggcgtgcagtgtgacgatgctgccacttactactgtcaacagggtgctagaagtagtaatgttgataatattttcggcggagggaccgaggtggtggtcaaaggtgatccagttgca

SEQ ID NO: 330 - DNA, 378 bp, Artificial Sequence
  Monoclonal antibody segment
acaggtgtccactcgcagtcgttggaggagtccgggggtcgcctggtaacgcctggaggatccctgacactcacctgcacagtctctggaatcgacctcagtagttatacaatgggctgggtccgccaggctccagggaaggggctggaatggatcggagtcattgctattaatggtggcacagtctacgcgagctgggcgaaaggccgattcaccatctccaaaacctcgaccacggtggatctgaaaatgaccagtccgacacccgaggacacggccacctatttctgtgccagaggtgctggtaaaacttggtcctcctttaacttgtggggccaaggcaccctggtcaccatctcctcagggcaacctaaggct

SEQ ID NO: 331 - DNA, 354 bp, Artificial Sequence
  Monoclonal antibody segment
acagatgccagatgcgagctcgtgctgacccagacaccagcctccgtgtctgaacctgtgggaggcacagtcaccatcaagtgccaggccagtcagagcattggtagtaatttagcctggtatcagcagaaaccagggcagcctcccaagctcctgatctacagggcatccactctggaatctggggtcccatcgcggttcagcggcagtggatctgggacagagttcactctcaccatcagcgacctggagtgtgccgatgctgccacttactactgtcaaagctattatggtagtagtactaatgctttcggcggagggaccgaggtggtggtcaaaggtgatccagttgca

SEQ ID NO: 332 - DNA, 378 bp, Artificial Sequence
  Monoclonal antibody segment
acaggtgtccactcgcagtcggtgaaggagtccgggggtcgcctggtcaagcctgacgaaaccctgacaatcacctgcacagtgtctggaatcgacctcagtcgctacagcatacagtgggtccgccaggctccagggggggggctgcaatggatcggtgtcatgagtatcggtagtagcgcatactacgcgaactgggcgaaaggccgattcaccatctccaaaacctcgtcgaccacggtggatctgacaatgaccagtctgacagccgcggacacggccacctatttctgtgccagaagtaatctctaccggggggcttttgatccctggggcccaggcaccctggtcaccatctcttcagggcaacctaaggct

SEQ ID NO: 333 - DNA, 370 bp, Artificial Sequence
  Monoclonal antibody segment
acagatgccagatgcgagctcgtgatgacccagacaccagcctccgtggaggcagctgtgggaggcacagtcaccatcaagtgccagtccagtgagagtgtttataagaataactacttatcctggtatcaacttaaaccagggcagcctcccaaactcctcttctattctgcttccactctggcatctagggtcccatcgcggttcagcggcagtggatctgggacacagttcactctcaccatcagcgacctggagtgtgacgatgctgccacttactactgtgcagggtattatggtagtagtagtgttgatcgttacactttcggcggagggaccgaggtggaaatcaaaggtgatccagttgcac

SEQ ID NO: 334 - DNA, 393 bp, Artificial Sequence
  Monoclonal antibody segment
acaggtgtccactcggagcagctgatggagtccgggggaggcctggtccagcctggtgcatccctgacactcacctgcaaagcctctggattctccttcagtagcagccaggacatgtgctgggtccgccaggctccagggaaggggcctgagtggatcggatgcattggttttggtagtggtaacacatggtacgcgagctgggtgaatggccgattcaccatctccagaagcaccagcctaaacacggtggagctgaaaatgaccggtctgacagccgcggacacggccacctatttctgtgcgagagatccatttactgttgctggtgatggtgataatttgtggggcccaggcaccctggtcaccgtctcctcagggcaacctaaggct

SEQ ID NO: 335 - DNA, 360 bp, Artificial Sequence
  Monoclonal antibody segment
acagatgccagatgcgagctcgatctgacccagacaccagcctccgtgtctgaacctgtgggaggcacagtcaccatcaagtgccaggccagtcagaccattaacaatgcattagcctggtatcagcagaaaccagggcagcctcccaagctcctgatctattctgcatccactctggcatctggggtctcatcgcggttcagcggcagtggatcagggacacagttcactctcaccatcagtggcgtggagtgtgacgatgctgccacttactactgtgcaggctatagaccttatattaatgatggtagtgctttcggcggagggaccgaggtggtggtcaaaggtgatccagttgca

SEQ ID NO: 336 - DNA, 390 bp, Artificial Sequence
  Monoclonal antibody segment
acaggtgtccactcggagcagtcggtggaggagtccgggggtcgcctggtcacgcctgggacacccctgacactcacctgcacagtctctggattctccctcagtagttatgcaatgagctgggtccgccaggctccagggaaggggctggagtggatcggagtcattgatgttactggtaggacatactacgcgagctgggcgaaaggccgattcaccatctccaaaacctcgaccacggtggatctgaaaatcaccagtccgacaaccgaggacacggccacctatttctgtggcagagatagctaccttgactatggtgaccccaatggtcttgatccctggggcccaggcaccctggtcaccatctcctcagggcaacctaaggct

SEQ ID NO: 337 - DNA, 367 bp, Artificial Sequence
  Monoclonal antibody segment
acagatgccagatgcgagctcgtgatgacccagacaccagcctccgtggaggcagctgtgggaggcacagtcaccatcaagtgccaggccagtcagagcattggtagtaatttagcctggtatcagcagaaaccagggcagcctcccaagctcctgatctatactgcatccaatctggcgcctggagtctcatcgcggttcaaaggcagtagatctgggacagagtccactctcatcatcagcgacctggagtgtgccgatgctgccacttactactgtcaatgtacttattatggtactagttatatggaattaggggctttcggcggagggaccgaggtggtggtcaaaggtgatccagttgcac

SEQ ID NO: 338 - DNA, 378 bp, Artificial Sequence
  Monoclonal antibody segment
acaggtgtccactcgcagtcggtgaaggagtccgggggtcgcctggtcacgcctgggacacccctgacactcacctgcacagtctctggattctccatcaatgacttccacatgtgctgggtccgccaggctccagggaaggggctggaatacatcggaatgattggtactggtggtagcgcatactacgcgacctggacaaaaggccgattcaccatctccagaacctcgaccacggtggatctaaagatcaccagtccgacaaccgaggacacggccacctatttctgtgccagacgggattggagtgatggtggttattcgctattatggggccaaggcaccctggtcaccgtctcttcagggcaacctaaggct

SEQ ID NO: 339 - DNA, 360 bp, Artificial Sequence
  Monoclonal antibody segment
acagatgccagatgcgagctcgatctgacccagacaccagcctccgtgtctgaacctgtgggaggcacagtcaccatcaagtgccaggccagtcagagcattcgcacctacttaaattggtatcagcagaaaccagggcagcctcccaaactcctgatctattctgcatccactctggcatctgggatcccatcgcggttcaaaggcagtggatctgggacacagttcactctcaccatcagcgacctggagtgtgccgatgctgccacttattactgtcaacagggatattattggagtgatgttgagaatactttcggcggagggaccgaggtggaaatcaacggtgatccagttgca

SEQ ID NO: 340 - DNA, 363 bp, Artificial Sequence
  Monoclonal antibody segment
acaggtgtccactcgcagtcggtgaaggagtccgggggtcgcctggtcacgcctgggacacccctgacactcacctgcaccgtctctggattctccctcagtatcaatgcaataaactgggtccgccaggctccagggaaggggctagaatggatcgggatcattactaatattggtaacacatactacgcgagctgggcaaaaggccgattcaccatctccagaacctcgaccacggtggatctgacaatgaccagtctgacaaccgaggacacggccacctatttctgtgccagaaactcttattttgatccctggggcccaggcaccctggtcaccatctcttcagggcaacctaaggct

SEQ ID NO: 341 - DNA, 360 bp, Artificial Sequence
  Monoclonal antibody segment
acagatgccagatgcgagctcgtgctgacccagacaccatcccccgtgtctgcagctgtgggaggcacagtcagcatcagttgccaggccagtcaaagtgttgatagtaataactactgttcctggtatcagcagaaaccagggcagcctcccaagctcctgatccacaaggcatccaaactggcatctggggtcccatcgcggttcaaaggcagtggatctgggacacagttcactctcaccatcaacgacctggagtgtgacgatgctgccacttactactgtgcaggcggtttgggtgataatatttatgctttcggcggagggaccgaggtggaaatcaacggtgatccagttgca

SEQ ID NO: 342 - DNA, 375 bp, Artificial Sequence
  Monoclonal antibody segment
acaggtgtccactcgcagtcggtgaaggagtccgggggtcgcctggtcacgcctgggacacccctgacactcacctgcacagtctctacaatcgacgtcagtaagtatgcagtgatctgggtccgccaggctccagggaaggggctggaatacatcggaatcattagtagtagtggtagcacatactacgcgagctgggcgaaaggccgattcaccatctccaaaacctcatcgaccacggtggatctgaaaatgaccagtccgacagccgaggacacggccacctatttctgtgccagagggggtaatggtgtcgcctttaaattatggggccaaggcaccctggtcaccatctcctcagggcaacctaaggct

SEQ ID NO: 343 - DNA, 360 bp, Artificial Sequence
  Monoclonal antibody segment
acagatgccagatgcgagctcgatctgacccagacaccagcctccgtggaggcagctgtgggaggcacagtcaccatcaattgccaggccagtcagagcatttacagctctttagcctggtatcagcagaaaccagggcagcctcccaaactcctgatctacgaaacatccaaagtggaatctggggtcccatcgcggttcagcggcagtggatctgggacacagttcactctcaccatcagcgacctggagtgtgccgatgctgccacttactactgtcaaagccatgattatattagtagtagtggtatgcctttcggcggagggaccgagctggagatcctaggtgatccagttgca

SEQ ID NO: 344 - DNA, 20 bp, Artificial Sequence
  Synthetic polynucleotide
ctgcttgttctcctgagccc

SEQ ID NO: 345 - DNA, 20 bp, Artificial Sequence
  Synthetic polynucleotide
tcaaagggtcagggttgagc

SEQ ID NO: 346 - DNA, 21 bp, Artificial Sequence
  Synthetic polynucleotide
gccctttggtgactttatgga

SEQ ID NO: 347 - DNA, 20 bp, Artificial Sequence
  Synthetic polynucleotide
gcagcaggttgtcttggatg

SEQ ID NO: 348 - DNA, 20 bp, Artificial Sequence
  Synthetic polynucleotide
gagaactgttatggggctat

SEQ ID NO: 349 - DNA, 20 bp, Artificial Sequence
  Synthetic polynucleotide
ttcaactggagaggcaaagg

SEQ ID NO: 350 - DNA, 25 bp, Artificial Sequence
  Synthetic polynucleotide
cgcataaccagtgaaacagcattgc

SEQ ID NO: 351 - DNA, 24 bp, Artificial Sequence
  Synthetic polynucleotide
ccctgtgctcagacagaaatgaga

SEQ ID NO: 352 - protein, 116 aa, Artificial sequence
  Synthetic antibody
QVQLVESGGGVVQPGRSLRLSCAASGIDLSDYYMTWVRQAPGKGLEYIGIIYASGNAYFASWAKGRFTISRDSSTNTLFLQMNSLRAEDTAVYYCARNIGGSSNLWGQGTLVTVSS

SEQ ID NO: 353 - protein, 116 aa, Artificial sequence
  Synthetic antibody
QVQLVESGGGVVQPGRSLRLSCAASGIDLSDYYMTWVRQAPGKGLEYIGIIYASGNAYFASWAKGRFTISRDNSTNTVFLQMNSLRAEDTAVYYCARNIGGSSNLWGQGTLVTVSS

SEQ ID NO: 354 - protein, 110 aa, Artificial sequence
  Synthetic antibody
AIQLTQSPSSLSASVGDRVTITCQASERIYNNLAWYQQKPGKAPKLLFYSASTLASGVPSRFSGSGSGTDFTLTISSLQPEDFATYYCQCTGYGTTYGLVFGGGTKVEIK

SEQ ID NO: 355 - protein, 110 aa, Artificial sequence
  Synthetic antibody
ELQLTQTPSSLSASVGDRVTIKCQASERIYNNLAWYQQKPGKAPKLLFYSASTLASGVPSRFSGSGSGTDFTLTISSLQPEDFATYYCQCTGYGTTYGLVFGGGTKVEIK

SEQ ID NO: 356 - DNA, 348 bp, Artificial sequence
  Synthetic antibody
caagtgcagctggtggagagcggcggaggcgtagtgcaacccggcagaagcctgagactgagctgcgccgctagcggcatcgacctgagcgactactacatgacctgggtgagacaagcccccggcaagggcctggagtacatcggcatcatctacgctagcggcaacgcctacttcgctagctgggccaagggcagattcaccatcagcagagacagcagcaccaacaccctgttcctgcagatgaacagcctgagagccgaggacaccgccgtgtactactgcgctagaaacatcggcggcagcagcaacctgtggggccaaggcaccctggtgaccgtgagcagc

SEQ ID NO: 357 - DNA, 348 bp, Artificial sequence
  Synthetic antibody
caagtgcagctggtggagagcggcggaggcgtagtgcaacccggcagaagcctgagactgagctgcgccgctagcggcatcgacctgagcgactactacatgacctgggtgagacaagcccccggcaagggcctggagtacatcggcatcatctacgctagcggcaacgcctacttcgctagctgggccaagggcagattcaccatcagcagagacaacagcaccaacaccgtgttcctgcagatgaacagcctgagagccgaggacaccgccgtgtactactgcgctagaaacatcggcggcagcagcaacctgtggggccaaggcaccctggtgaccgtgagcagc

SEQ ID NO: 358 - DNA, 330 bp, Artificial sequence
  Synthetic antibody
gccattcagctgacacagagccctagcagcctgagcgctagcgtgggcgacagagtgaccatcacctgccaagctagcgagagaatctacaacaacctggcctggtatcagcagaagcccggcaaggcccccaagctgctgttctacagcgctagcaccctggctagcggcgtgcctagcagattcagcggcagcggcagcggcaccgacttcaccctgaccatcagcagcctgcagcccgaggacttcgccacctactactgtcagtgcaccggctacggcaccacctacggcctggtgttcggcggcggcaccaaggtggagatcaag

SEQ ID NO: 359 - DNA, 330 bp, Artificial sequence
  Synthetic antibody
gagctgcagctgacacagacccctagcagcctgagcgctagcgtgggcgacagagtgaccatcaagtgccaagctagcgagagaatctacaacaacctggcctggtatcagcagaagcccggcaaggcccccaagctgctgttctacagcgctagcaccctggctagcggcgtgcctagcagattcagcggcagcggcagcggcaccgacttcaccctgaccatcagcagcctgcagcccgaggacttcgccacctactactgtcagtgcaccggctacggcaccacctacggcctggtgttcggcggcggcaccaaggtggagatcaag

SEQ ID NO: 360 - DNA, 754 bp, Artificial sequence
  Synthetic promoter
ataacaaatctgtatattggaccctctgcttagcagtgagaaagcaggtttgaagacaataaagccaggcctgtatgtggagattggcaccatgaccctggattatgttttatgggatatgcctccaagggcaactctgcatttatcccatggcctctggcttccaaattttctaggcacttccctctggggtaaaggaatcaaaattggcttatttcccttgcaaaccatccttggcgactaagtactgccttgtcttctactgagagatggccatatttttggcacctgcctttccttgtgggaggtgtttcctctgtttacatagcaaaggggctgtaggagagacagacggagctggacttacagcagaaccaagtcatgatagcctgtttctattttgttttcagcatttttccgccagttctggccacctcctttcctctggaacatgctgatttcagcaagtccagctctgtcagctctgcccccccagtctattggtttctgatcatctgataatgctttgcctgcactcaggacctgtctttgtccctcctcttaacatacttgcagctaaaactaaatattgctgcttggggacctccttctagccttaaatttcagctcatcaccttcacctgccttggtcatggctctgctattctccttgatccttggtgagtatctctgcacctgttggtttaggcttcagagttttctggcactttgattaggagaactttctccccgc

SEQ ID NO: 361 - DNA, 15 bp, Artificial sequence
  Synthetic promoter
gtcagctctgccccc

(19) United States
(12) Patent Application Publication
(10) Pub. No.: US 2024/0383999 A1
(43) Pub. Date: Nov. 21, 2024

(54) CD5L-BINDING ANTIBODIES AND USES FOR THE SAME

(71) Applicant: THE BOARD OF REGENTS OF THE UNIVERSITY OF TEXAS SYSTEM, Austin, TX (US)

(72) Inventors: Ningyan ZHANG, Houston, TX (US); Zhiqiang AN, Houston, TX (US); Zhiqiang KU, Houston, TX (US); Hui DENG, Houston, TX (US); Lingegowda S MANGALA, Houston, TX (US); Anil K. SOOD, Houston, TX (US)

(73) Assignee: THE BOARD OF REGENTS OF THE UNIVERSITY OF TEXAS SYSTEM, Austin, TX (US)

(21) Appl. No.: 17/995,214

(22) PCT Filed: Apr. 1, 2021
(86) PCT No.: PCT/US2021/025429

§ 371 Date: Sep. 30, 2022

Related U.S. Application Data
(60) Provisional application No. 63/004,149, filed on Apr. 2, 2020.

(51) Int. Cl. C07K 16/28; A61K 39/00

(52) U.S. Cl. CPC C07K 16/2896; A61K 2039/505; C07K 2317/24; C07K 2317/92

(57) ABSTRACT

Provided herein are anti-CD5L monoclonal antibodies. Further provided herein are methods of use thereof for the treatment of a disease or disorder, such as cancer.

### PRIORITY CLAIM

This application claims benefit of priority to U.S. Provisional Application Ser. No. 63/004,149, filed Apr. 2, 2020, the entire contents of which are hereby incorporated by reference.

### STATEMENT REGARDING FEDERALLY SPONSORED RESEARCH

This invention was made with government support under Grant No. CA217685 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 1, 2021, is named UTFCP1501WO_ST25.txt and is 183 kilobytes in size.

### BACKGROUND

### 1. Field

The present disclosure relates generally to the fields of cancer biology and medicine. More particularly, it concerns anti-CD5L monoclonal antibodies and methods of use thereof.

### 2. Description of Related Art

Angiogenesis is known to play an important role in tumor development and growth (Folkman, 1971). This complex process relies on the careful orchestration of many factors including vascular endothelial growth factor (VEGF) and its receptor (VEGFR), fibroblast growth factor (FGF), and others (Weis and Cheresh, 2011). Many anti-angiogenesis drugs, particularly focused on the VEGF/VEGFR pathway, have been developed and approved for cancer treatment. While many patients benefit from such therapies, virtually all patients will eventually develop relapse or progression of disease. Understanding and overcoming adaptive changes to anti-VEGF drugs represents an opportunity to further enhance the efficacy of these drugs and potentially delay or prevent adaptive resistance (Bergers and Hanahan, 2008).

### SUMMARY

In one embodiment, provided herein are monoclonal antibodies or antibody fragments, wherein the antibodies or antibody fragments are characterized by clone-paired heavy and light chain CDR sequences from Tables 1 and 2.

In some aspects, said antibodies or antibody fragments are encoded by light and heavy chain variable sequences according to clone-paired sequences from Table 4. In some aspects, said antibodies or antibody fragments are encoded by light and heavy chain variable sequences having at least 70%, 80%, or 90% identity to clone-paired sequences from Table 4. In some aspects, said antibodies or antibody fragments are encoded by light and heavy chain variable sequences having at least 95% identity to clone-paired sequences from Table 4.

In some aspects, said antibodies or antibody fragments comprise light and heavy chain variable sequences according to clone-paired sequences from Table 3. In some aspects, said antibodies or antibody fragments comprise light and heavy chain variable sequences having at least 70%, 80%, or 90% identity to clone-paired variable sequences from Table 3. In some aspects, said antibodies or antibody fragments comprise light and heavy chain variable sequences having 95% identity to clone-paired sequences from Table 3.

In some aspects, the antibodies or antibody fragments comprise:
a heavy chain variable region (VH) comprising a VHCDR1 amino acid sequence of SEQ ID NO: 81, a VHCDR2 amino acid sequence of SEQ ID NO: 82, and a VHCDR3 amino acid sequence of SEQ ID NO: 84; and a light chain variable region (VL) comprising a VLCDR1 amino acid sequence of SEQ ID NO: 4, a VLCDR2 amino acid sequence of SEQ ID NO: 2, and a VLCDR3 amino acid sequence of SEQ ID NO: 5;
a heavy chain variable region (VH) comprising a VHCDR1 amino acid sequence of SEQ ID NO: 87, a VHCDR2 amino acid sequence of SEQ ID NO: 88, and a VHCDR3 amino acid sequence of SEQ ID NO: 89; and a light chain variable region (VL) comprising a VLCDR1 amino acid sequence of SEQ ID NO: 1, a VLCDR2 amino acid sequence of SEQ ID NO: 2, and a VLCDR3 amino acid sequence of SEQ ID NO: 6;
a heavy chain variable region (VH) comprising a VHCDR1 amino acid sequence of SEQ ID NO: 160, a VHCDR2 amino acid sequence of SEQ ID NO: 161, and a VHCDR3 amino acid sequence of SEQ ID NO: 162; and a light chain variable region (VL) comprising a VLCDR1 amino acid sequence of SEQ ID NO: 67, a VLCDR2 amino acid sequence of SEQ ID NO: 10, and a VLCDR3 amino acid sequence of SEQ ID NO: 68; or
a heavy chain variable region (VH) comprising a VHCDR1 amino acid sequence of SEQ ID NO: 178, a VHCDR2 amino acid sequence of SEQ ID NO: 179, and a VHCDR3 amino acid sequence of SEQ ID NO: 180; and a light chain variable region (VL) comprising a VLCDR1 amino acid sequence of SEQ ID NO: 77, a VLCDR2 amino acid sequence of SEQ ID NO: 10, and a VLCDR3 amino acid sequence of SEQ ID NO: 78.

In some aspects, said antibodies or antibody fragments are encoded by a heavy chain variable sequence having at least 70%, 80%, or 90% identity to SEQ ID NO: 266 and a light chain variable sequence having at least 70%, 80%, or 90% identity to SEQ ID NO: 267. In some aspects, said antibodies or antibody fragments are encoded by a heavy chain variable sequence having at least 95% identity to SEQ ID NO: 266 and a light chain variable sequence having at least 95% identity to SEQ ID NO: 267. In some aspects, said antibodies or antibody fragments are encoded by a heavy chain variable sequence according to SEQ ID NO: 266 and a light chain variable sequence according to SEQ ID NO: 267.

In some aspects, said antibodies or antibody fragments comprise a heavy chain variable sequence having at least 70%, 80%, or 90% identity to SEQ ID NO: 185 and a light chain variable sequence having at least 70%, 80%, or 90% identity to SEQ ID NO: 186. In some aspects, said antibodies or antibody fragments comprise a heavy chain variable sequence having at least 95% identity to SEQ ID NO: 185 and a light chain variable sequence having at least 95% identity to SEQ ID NO: 186. In some aspects, said antibodies or antibody fragments comprise a heavy chain variable sequence having a sequence according to SEQ ID NO: 185 and a light chain variable sequence having a sequence according to SEQ ID NO: 186.

In some aspects, said antibodies or antibody fragments are encoded by a heavy chain variable sequence having at least 70%, 80%, or 90% identity to SEQ ID NO: 268 and a light chain variable sequence having at least 70%, 80%, or 90% identity to SEQ ID NO: 269. In some aspects, said antibodies or antibody fragments are encoded by a heavy chain variable sequence having at least 95% identity to SEQ ID NO: 268 and a light chain variable sequence having at least 95% identity to SEQ ID NO: 269. In some aspects, said antibodies or antibody fragments are encoded by a heavy chain variable sequence according to SEQ ID NO: 268 and a light chain variable sequence according to SEQ ID NO: 269.

In some aspects, said antibodies or antibody fragments comprise a heavy chain variable sequence having at least 70%, 80%, or 90% identity to SEQ ID NO: 188 and a light chain variable sequence having at least 70%, 80%, or 90% identity to SEQ ID NO: 189. In some aspects, said antibodies or antibody fragments comprise a heavy chain variable sequence having at least 95% identity to SEQ ID NO: 188 and a light chain variable sequence having at least 95% identity to SEQ ID NO: 189. In some aspects, said antibodies or antibody fragments comprises a heavy chain variable sequence having a sequence according to SEQ ID NO: 188 and a light chain variable sequence having a sequence according to SEQ ID NO: 189.

In some aspects, said antibodies or antibody fragments are encoded by a heavy chain variable sequence having at least 70%, 80%, or 90% identity to SEQ ID NO: 324 and a light chain variable sequence having at least 70%, 80%, or 90% identity to SEQ ID NO: 325. In some aspects, said antibodies or antibody fragments are encoded by a heavy chain variable sequence having at least 95% identity to SEQ ID NO: 324 and a light chain variable sequence having at least 95% identity to SEQ ID NO: 325. In some aspects, said antibodies or antibody fragments are encoded by a heavy chain variable sequence according to SEQ ID NO: 324 and a light chain variable sequence according to SEQ ID NO: 325.

In some aspects, said antibodies or antibody fragments comprise a heavy chain variable sequence having at least 70%, 80%, or 90% identity to SEQ ID NO: 244 and a light chain variable sequence having at least 70%, 80%, or 90% identity to SEQ ID NO: 245. In some aspects, said antibodies or antibody fragments comprise a heavy chain variable sequence having at least 95% identity to SEQ ID NO: 244 and a light chain variable sequence having at least 95% identity to SEQ ID NO: 245. In some aspects, said antibodies or antibody fragments comprise a heavy chain variable sequence having a sequence according to SEQ ID NO: 244 and a light chain variable sequence having a sequence according to SEQ ID NO: 245.

In some aspects, said antibodies or antibody fragments are encoded by a heavy chain variable sequence having at least 70%, 80%, or 90% identity to SEQ ID NO: 338 and a light chain variable sequence having at least 70%, 80%, or 90% identity to SEQ ID NO: 339. In some aspects, said antibodies or antibody fragments are encoded by a heavy chain variable sequence having at least 95% identity to SEQ ID NO: 338 and a light chain variable sequence having at least 95% identity to SEQ ID NO: 339. In some aspects, said antibodies or antibody fragments is encoded by a heavy chain variable sequence according to SEQ ID NO: 338 and a light chain variable sequence according to SEQ ID NO: 339.

In some aspects, said antibodies or antibody fragments comprise a heavy chain variable sequence having at least 70%, 80%, or 90% identity to SEQ ID NO: 258 and a light chain variable sequence having at least 70%, 80%, or 90% identity to SEQ ID NO: 259. In some aspects, said antibodies or antibody fragments comprise a heavy chain variable sequence having at least 95% identity to SEQ ID NO: 258 and a light chain variable sequence having at least 95% identity to SEQ ID NO: 259. In some aspects, said antibodies or antibody fragments comprises a heavy chain variable sequence having a sequence according to SEQ ID NO: 258 and a light chain variable sequence having a sequence according to SEQ ID NO: 259.

In some aspects of any of the present embodiments, said antibodies or antibody fragments are humanized antibodies such as wherein said antibody or antibody fragment comprises a heavy chain variable sequence having a sequence according to SEQ ID NO: 352 and a light chain variable sequence having a sequence according to SEQ ID NO: 354; a heavy chain variable sequence having a sequence according to SEQ ID NO: 352 and a light chain variable sequence having a sequence according to SEQ ID NO: 355; a heavy chain variable sequence having a sequence according to SEQ ID NO: 353 and a light chain variable sequence having a sequence according to SEQ ID NO: 354; or a heavy chain variable sequence having a sequence according to SEQ ID NO: 353 and a light chain variable sequence having a sequence according to SEQ ID NO: 355.

In some aspects of any of the present embodiments, the antibody fragments are monovalent scFv (single chain fragment variable) antibodies, divalent scFvs, Fab fragments, F(ab′)₂ fragments, F(ab′)₃ fragments, Fv fragments, or single chain antibodies. In some aspects of any of the present embodiments, said antibodies are chimeric antibodies or bispecific antibodies. In some aspects, said antibodies are IgG antibodies or recombinant IgG antibodies or antibody fragments. In some aspects of any of the present embodiments, the antibodies or antibody fragments are conjugated or fused to an imaging agent or a cytotoxic agent.

In one embodiment, provided herein are monoclonal antibodies or antigen binding fragments thereof, which compete for binding to the same epitope as the monoclonal antibodies or antigen-binding fragments thereof according to any one of the present embodiments. In some aspects, said antibody or antibody fragment is a humanized antibody. In some aspects, the antibody fragment is a monovalent scFv (single chain fragment variable) antibody, divalent scFv, Fab fragment, F(ab′)₂ fragment, F(ab′)₃ fragment, Fv fragment, or single chain antibody. In some aspects, said antibody is a chimeric antibody or bispecific antibody. In some aspects, said antibody is an IgG antibody or a recombinant IgG antibody or antibody fragment. In some aspects, the antibody is conjugated or fused to an imaging agent or a cytotoxic agent.

In one embodiment, provided herein are compositions comprising a therapeutically effective amount of an antibody or antibody fragment of any one of the present embodiments, and a pharmaceutically acceptable diluent or vehicle. In some aspects, the compositions are provided for use as a medicament for treating a cancer in a patient.

In one embodiment, provided herein are hybridomas or engineered cells encoding an antibody or antibody fragment of any one of the present embodiments.

In one embodiment, provided herein are methods of treating a patient having a cancer, the methods comprising administering an effective amount of an anti-CD5L antibody or antibody fragment. In some aspects, the anti-CD5L antibody or antibody fragment is the antibody or antibody fragment of any one of the present embodiments.

In some aspects, the cancer is breast cancer, colon cancer, prostate cancer, lung cancer, gastric cancer, ovarian cancer, endometrial cancer, renal cancer, hepatocellular cancer, thyroid cancer, uterine cancer, esophageal carcinoma, squamous cell carcinoma, leukemia, osteosarcoma, melanoma, glioblastoma or neuroblastoma. In some aspects, the cancer is ovarian cancer.

In some aspects, the patient is further administered a second anticancer therapy. The second anticancer therapy may be an anti-angiogenic therapy. The anti-angiogenic therapy may be an anti-VEGF therapy. In some aspects, the anti-CD5L antibody or antibody fragment results in increased efficacy of the anti-angiogenic therapy. In some aspects, the second anticancer therapy comprises chemotherapy, immunotherapy, surgery, radiotherapy, or biotherapy.

In some aspects, the anti-CD5L antibody or antibody fragment is administered intravenously. In some aspects, the patient's cancer has been determined to overexpress CD5L. In some aspects, the patient's serum or plasma has been determined to have an elevated level of CD5L. In some aspects, the patient's cancer has recurred following anti-angiogenic therapy. In some aspects, the patient's cancer has been determined to be resistant to anti-VEGF therapy.

In one embodiment, provided herein are methods for the detection of CD5L comprising (a) incubating a sample with an anti-CD5L antibody or antibody fragment of any of the present embodiments; and (b) measuring the binding of the anti-CD5L antibody or antibody fragment to the sample. In some aspects, the sample is blood, serum, plasma, saliva, biopsy, urine, or cerebrospinal fluid.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1M. Upregulation of CD5L in anti-VEGF Therapy Resistant Endothelial Cells Promotes Angiogenesis and Migration. (FIG. 1A) Schematic representing time points at which tumors isolated during course of B20 treatment. Tumor progression identified by increase in bioluminescence. (FIG. 1B) Heat map from gene expression profile demonstrating fold change in B20 resistant compared to sensitive tumors. (FIG. 1C) Fold change of RNA expression of select genes in both B20 sensitive and resistant tumors. (FIG. 1D) CD5L staining in endothelial cells from mouse tumors sensitive (L) and resistant (R) to B20. (FIG. 1E) CD5L protein expression in RF24 endothelial cells containing CD5L overexpressing plasmid versus empty vector (EV). (FIG. 1F) Cell proliferation in RF24 endothelial cells containing CD5L overexpressing plasmid versus EV. (FIGS. 1G&1H) Tube formation (FIG. 1G) and cell migration (FIG. 1H) in RF24 endothelial cells containing CD5L overexpressing plasmid versus EV. (FIG. 1I) Concentration of CD5L in media collected from RF24 endothelial cells containing CD5L overexpressing plasmid. (FIGS. 1J-1M) CD5L protein expression (FIG. 1J), cell proliferation (FIG. 1K), tube formation (FIG. 1L), and cell migration (FIG. 1M) in RF24 endothelial cells treated with siCD5L versus siControl. (*P<0.05, **P<0.01, ***P<0.001). Bars represent mean±sd. EV-empty vector.

FIGS. 2A-2N. CD5L is Upregulated Through Hypoxia-Induced PPAR-γ Overexpression. (FIGS. 2A&2B) CD5L mRNA (FIG. 2A) and protein expression (FIG. 2B) in RF24 endothelial cells containing PPAR-γ overexpressing plasmid versus (EV). (FIG. 2C) CD5L promoter construct activation using either RF24 endothelial cells containing PPAR-γ overexpressing plasmid versus (EV). (FIGS. 2D&2E) PPAR-γ and CD5L mRNA (FIG. 2D) and protein (FIG. 2E) expression in RF24 endothelial cells treated with siPPAR-γ versus siControl. (FIG. 2F) CD5L promoter construct activation using RF24 endothelial cells treated with siPPAR-γ versus siControl. (FIG. 2G) Luciferase expression in RF24 endothelial cells after co-transfection of PPAR-γ overexpressing plasmid and CD5L promoter construct harboring mutated PPAR-γ binding site. (FIGS. 2H&2I) PPAR-γ and CD5L mRNA (FIG. 2H) and protein (FIG. 2I) expression in RF24 endothelial cells cultured in hypoxic or normoxic conditions. (FIGS. 2J&2K) PPAR-γ and CD5L mRNA (FIG. 2J) and protein (FIG. 2K) expression in RF24 endothelial cells treated for 6 and 30 hours with cobalt chloride (HIF1α stabilizer). (FIG. 2L) PPAR-γ and CD5L mRNA expression in RF24 endothelial cells treated with YC-1 or topotecan under hypoxic conditions. (FIG. 2M) CD5L WT promoter construct activation in RF24 endothelial cells cultured in hypoxic and normoxic conditions. (FIG. 2N) Chromatin immunoprecipitation (ChIP) analysis of the CD5L promoter using an anti-PPAR-γ antibody under hypoxic and normoxic conditions. (*P<0.05, **P<0.01, ***P<0.001). Bars represent mean±sd.

FIGS. 3A-3J. Exogenous CD5L Treatment of RF24 Endothelial Cells Results in Upregulation of PI3K/AKT Signaling. (FIG. 3A) Reverse phase protein array (RPPA) analysis of RF24 endothelial cells treated with CD5L protein versus control. (FIG. 3B) AKT pathway activation measured by phospho-AKT/AKT in RF24 cells after exogenous CD5L protein treatment. (FIGS. 3C-3E) AKT pathway activation (FIG. 3C), tube formation (FIG. 3D), and cell migration (FIG. 3E) in RF24 cells treated with CD5L protein and either LY294002 (PI3K inhibitor) or DMSO. (FIG. 3F) CD36 mRNA expression in RF24 cells treated with CD5L protein. (FIG. 3G) AKT pathway activation in RF24 cells treated with siCD36. (FIG. 3H) PPAR-γ mRNA expression in RF24 cells treated with CD5L protein. (FIGS. 3I&3J) Cell viability of RF24 cells at increasing concentrations of bevacizumab with the additional of either CD5L protein (FIG. 3I) or siCD5L (FIG. 3J). (ns—not significant, *P<0.05, **P<0.01, ***P<0.001). Bars represent mean±sd.

FIGS. 4A-4G. PPAR-γ Silencing Inhibits Tumor Growth and Angiogenesis in ID8 Xenograft Model. (FIG. 4A) Photographs of representative mice from each group in wild-type (WT) and TIE2CrePPARγflox/flox (KO) mice. (FIGS. 4B&4C) Tumor weight (g) and number of tumor nodules (mean±sd., as denoted by error bars). Significant difference in tumor weight is denoted by the asterisk (*P<0.05, **P<0.01, ***P<0.001). (FIGS. 4D&4E) Statistical analysis of paraffin slides for the expression of Ki67 and CD31. Five fields per slide and at least three slides (5 slides for each group, WT and KO) were examined. Bars represent mean±sd. **P<0.01 and ***P<0.001. (FIG. 4F) Survival plot for B20, anti-VEGF treatment. B20 was injected intraperitoneal twice weekly at a dose of 5 mg/kg. (FIG. 4G) Expression of p-AKT relative to AKT in tumor samples from WT versus TIE2CrePPARγflox/flox (KO) mice (p-AKT to AKT ratio determined after normalization of AKT to vinculin).

FIGS. 5A-5I. Novel Antibodies Targeting CD5L Exhibit Anti-Tumor and Anti-Angiogenic Effects. (FIG. 5A) Photographs of representative mice from both wild-type (WT) and anti-CD5L antibody R-35 treated groups. Mice were treated intraperitoneally with either PBS or anti-CD5L antibody (10 mg/kg) starting on D8 after tumor injection until D35. (FIGS. 5B&5C) Tumor weight (g; FIG. 5B)) and number of tumor nodules (mean±SEM, as denoted by error bars; FIG. 5C). (FIG. 5D) CD31 immunofluorescence staining of tumors from control vs. anti-CD5L antibody treated groups. (FIG. 5E&5F) Tube formation (FIG. 5E) and cell migration (FIG. 5F) of RF24 cells treated with either control antibody alone, control antibody+CD5L protein, or R-35 antibody+CD5L protein. (*P<0.05, **P<0.01, ***P<0.001). Bars represent mean±sd, aside from FIGS. 5B and 5C as mentioned above. (FIGS. 5G&5H) For tumor cells injection, SKOV3ip1 cells (1×10⁶) were injected intraperitoneally. For the anti-CD5L antibody treatment, control or experimental antibody was injected intraperitoneally once weekly at a dose of 10 mg/kg body weight. After mice were euthanized with CO₂, total tumor weight (FIG. 5H) and number of tumor nodules (FIG. 5G) were recorded. (FIG. 5I) Mouse body weights were measured at the end of in vivo experiments by weighing individual mouse on a balance.

FIGS. 6A-6D. CD5L Overexpression is Associated with Bevacizumab Resistance and Worse Overall Survival in Ovarian Cancer Patients. (FIGS. 6A&6B) CD5L protein expression measured by immunohistochemistry (IHC) (FIG. 6A) and serum protein levels (FIG. 6B) in ovarian cancer patients classified as either responsive or non-responsive to bevacizumab (mean±sd, as denoted by error bars). (FIG. 6C) Representative images of low (upper) and high (lower) CD5L protein expression in tumor endothelial cells from human ovarian cancer patients. (FIG. 6D) Kaplan Meier curve of overall survival in high-grade serous ovarian cancer patients stratified according to CD5L protein expression level, as measured by IHC (see FIG. 6C).

FIG. 7. Mechanism of CD5L Induced AVA Resistance. Anti-VEGF treatment may initially cause tumor regression via decreased angiogenesis (lower tumor—single blood vessel); however, adaptive resistance frequently emerges after time leading to tumor growth and increased angiogenesis (larger tumor with many blood vessels). Inset demonstrates tumor endothelial cell showing that local tumor hypoxia leads to an increase in CD5L secretion via overexpression of the transcription factor PPAR-γ. Secreted CD5L binds to the CD36 receptor causing an activation of the AKT pathway leading ultimately to increased cell proliferation and angiogenesis.

FIG. 8. Promoter Sequence of CD5L. PPAR-γ binding site identified in red. Lower image represents CD5L promoter construct with critical base pairs in PPAR-γ binding highlighted (CTCT). (SEQ ID NOS: 360 and 361)

FIG. 9. Ingenuity Pathway Analysis (IPA) of AVA Resistant Mouse Tumor Endothelial Cells. Resistance to anti-VEGF therapy is associated with increased hypoxia signaling. IPA analysis performed on gene expression profile presented in FIG. 1B.

FIGS. 10A-10B. Effect of Exogenous CD5L Protein Treatment on RF24 Endothelial Cells. Tube formation (FIG. 10A) and cell migration (FIG. 10B) of RF24 cells alone or after addition of 400 ng/ml CD5L protein. (**P<0.01, ***P<0.001). Bars represent mean±sd.

FIG. 11. ELISA titration to determine EC₅₀ of CD5L mAbs. Human CD5L protein (Sino Biologicals) was coated on a 96-well high binding plate over night at 4° C. in PBS. Purified mAbs were added at a series of antibody concentrations in 3-fold titration for binding to CD5L antigen coated on the plate. Bound antibody was detected using a secondary antibody against rabbit IgG conjugated with HRP and TMB substrate for detection of A450 nm. EC₅₀ was estimated using GraphPad software by applying a 4-parameter fitting to calculate binding affinity of the antibodies. Experiments have 3 repeats and error bars indicate standard deviation.

FIG. 12. Kinetic binding sensorgrams of CD5L antibodies. Kinetic binding constant (KD) of CD5L antibodies was determined using Octet BLI instrument according to manufacturer's instruction.

FIG. 13. Effect of CD5L Ab on toxicity of C57/BL6 mice.

FIG. 14. H&E staining of vital organs of IgG control and CD5L Ab treated C57/BL6 mice.

FIG. 15. CD31 staining in organs of CD5L Ab treated mice.

FIG. 16. Effects of CD5L Ab on survival of OVCAR8 tumor bearing mice.

FIG. 17. Binding affinity of the humanized CD5L-35Hu mAbs to CD5L using Bilayer Inferometry (BLI) Octet method.

### DETAILED DESCRIPTION

Anti-angiogenic treatment targeting the vascular endothelial growth factor (VEGF) pathway is a powerful tool to combat tumor growth and progression; however, drug-resistance frequently occurs. CD5 antigen-like precursor (CD5L) is identified herein as an important protein in the response to anti-angiogenic treatment leading to the emergence of adaptive resistance. Using a newly developed monoclonal antibody targeting CD5L, the pro-angiogenic effects of CD5L overexpression was abated in both in vitro and in vivo settings. Additionally, CD5L overexpression in cancer patients was shown to be associated with bevacizumab resistance and worse overall survival. These findings implicate CD5L as a major driver of adaptive resistance to anti-angiogenic therapy, and modalities to target CD5L have important clinical utility.

Anti-angiogenic therapies are an integral component in the treatment of cancer. While drugs such as bevacizumab, an anti-VEGF monoclonal antibody, are approved by the FDA for cancer therapy, a major limitation of anti-VEGF drugs is the high frequency of adaptive resistance, rendering treatment ineffective. This is particularly important as the indications for use of this drug expand. Herein, it was show that increased endothelial CD5L expression is an important driver of resistance to anti-VEGF therapy and that modalities used to negate its effect (e.g., an antibody) can be employed to reverse the resistant phenotype. Incorporation of anti-CD5L agents into clinical trials may reveal a feasible approach for lengthening the efficacy of anti-VEGF therapies.

To examine potential mechanisms underlying resistance to anti-VEGF antibody (AVA) therapy, mouse models were used to identify tumors that demonstrated growth subsequent to a period of initial response. Specifically, orthotopic mouse models of ovarian cancer designed to develop adaptive resistance after treatment with the anti-VEGF antibody B20, which targets both mouse and human VEGF-A, were established. The genomic profiles of tumor-associated endothelial cells collected at pre-treatment, maximal response, and at tumor progression were examined, and substantially elevated CD5L levels were found at the time of progression. CD5L (or AIM-apoptosis inhibitor expressed by macrophages) was previously identified as a soluble protein secreted primarily from macrophages in lymphoid tissues during an inflammatory response (Sanjurjo et al., 2015). While additional roles of CD5L have been discovered since, those related specifically to endothelial cells and angiogenesis remain unknown. Here, data are presented implicating CD5L expression in adaptive resistance to bevacizumab. It was also demonstrated that neutralizing CD5L using an antibody targeting CD5L blocked adaptive resistance to anti-angiogenic therapy, indicating anti-CD5L is a potential new therapeutic strategy for overcoming resistance to bevacizumab and other anti-angiogenesis therapies.

### I. ASPECTS OF THE DISCLOSURE

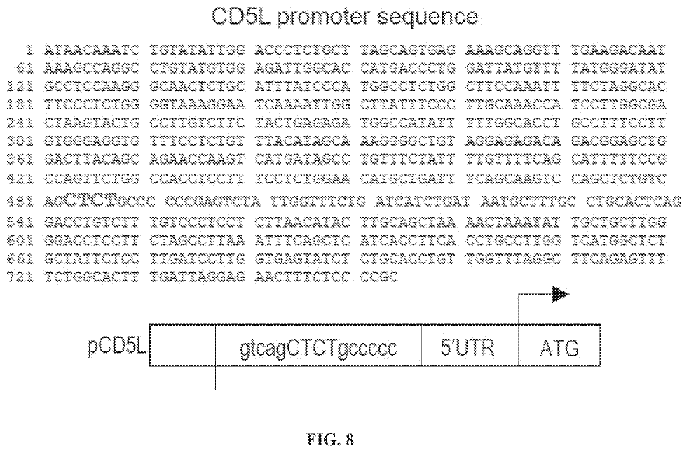

The data provided herein identify CD5L as an important mediator of AVA resistance, a previously unrecognized role which functions in promoting tumor angiogenesis. From a conceptual perspective, hypoxia incurred due to prolonged VEGF blockade ultimately drives the overexpression of CD5L through the upregulation of the transcription factor PPAR-γ. Through analysis of the downstream pathways, prominent activation of the PI3K/AKT pathway with increased CD5L signaling in tumor endothelial cells was demonstrated (FIG. 7). Importantly, blocking CD5L through multiple modalities could restore response to anti-VEGF therapy.

Anti-VEGF drugs are currently approved for the treatment of many different cancer types. Unfortunately, although the initial response rates are high with these therapies, most patients develop resistant disease within weeks-to-months. The mechanisms underlying such adaptive resistance are likely to be multi-factorial and are not fully understood. One such mechanism involves the upregulation of pro-angiogenic factors other than VEGF in response to AVA treatment, such as fibroblast growth factor 1 and ephrin A1 (Casanovas et al., 2005). Additionally, tumors treated with AVA compounds adapt by increasing their invasive potential without relying on novel vessel generation (Norden et al., 2008, Narayana et al., 2009). Regardless of the exact mechanism, the phenomenon that tumor endothelial cells adapt to their specific microenvironment is now well accepted. Therefore, a systematic approach was taken, whereby mRNA profiling from tumor endothelial cells from responder vs. non-responder mice was carried out and CD5L was identified as the mostly highly expressed mRNA in treatment-resistant endothelial cells.

Initially named for its anti-apoptotic role on leukocytes (Miyazaki et al., 1999), CD5L has since been implicated as a key regulator of inflammatory responses, particularly through its effect on macrophages. It has also been shown to be involved in a variety of cellular processes including atherosclerosis, infection, and cancer (Sanjurjo et al., 2015). Although CD5L is secreted primarily by macrophages, it has been shown to have diverse roles in the immune system. Mice deficient in CD5L have reduced lymphocytes in liver granulomas when challenged with heat-killed C. parvum compared to wild type mice (Kuwata et al., 2003). Additionally, in vitro studies using liver T- and natural killer T (NKT)-cells from mice exposed to C. parvum showed significant inhibition of apoptosis after treatment with recombinant CD5L (Kuwata et al., 2003). CD5L has also been shown to induce formation of bronchoalveolar adenocarcinoma in a transgenic mouse model with CD5L overexpressing myeloid cells (Qu et al., 2009). Interestingly, CD5L seems to have a protective role in mouse hepatocellular carcinoma through its interaction with CD55, CD59, and Crry, leading to subsequent complement activation and induced necrotic death of hepatocytes (Maehara et al., 2014). The scavenger receptor CD36 has been implicated as the primary cell-surface receptor for CD5L (Kurokawa et al., 2010) and although CD36 is expressed on endothelial cells, whether CD5L played an important role in endothelial survival was not known (Silverstein and Febbraio, 2009). Silencing CD36 prevented the pro-angiogenic phenotype associated with increased CD5L expression, iterating the necessity of both CD5L and intact CD36 to develop AVA resistance.

Through analysis of an ovarian cancer cohort, a clinical correlation between CD5L overexpression and bevacizumab resistance was demonstrated. This suggests that upregulation of CD5L by tumor endothelial cells is an integral component of the adaptive resistance mechanism against bevacizumab treatment. Concordantly, patients who had higher levels of CD5L also had worse overall survival, likely due to the decreased anti-tumor effect of bevacizumab seen in the resistant setting.

Adaptive resistance to anti-VEGF therapy is a complex mechanism programmed by tumors to allow for continued survival. As increasing data emerges regarding the molecular pathways responsible for this phenomenon, it will be important to carefully select the critical components for development of novel therapeutics and subsequent clinical trials. CD5L is herein identified as an integral protein in the adaptive response to anti-VEGF treatment and strategies aimed at targeting it could benefit patients being treated with anti-angiogenic drugs.

### II. DEFINITIONS

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv)), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (VH-CH1-VH-CH1) which, together with complementary light chain polypeptides, form a pair of antigen binding regions.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. For example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

A “constant region” of an antibody refers to the constant region of the antibody light chain or the constant region of the antibody heavy chain, either alone or in combination. The constant regions of the light chain (CL) and the heavy chain (CH1, CH2 or CH3, or CH4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody.

A “variable region” of an antibody refers to the variable region of the antibody light chain or the variable region of the antibody heavy chain, either alone or in combination. The variable regions of both the light (VL) and heavy (VH) chain portions determine antigen recognition and specificity. VL and VH each consist of four framework regions (FR) connected by three complementarity determining regions (CDRs) also known as hypervariable regions. The CDRs complement an antigen's shape and determine the antibody's affinity and specificity for the antigen. There are six CDRs in both VL and VH. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (the Kabat numbering scheme; see Kabat et al., Sequences of Proteins of Immunological Interest (5th ed., 1991, National Institutes of Health, Bethesda Md.)); and (2) an approach based on crystallographic studies of antigen-antibody complexes (the Chothia numbering scheme which corrects the sites of insertions and deletions (indels) in CDR-L1 and CDR-H1 suggested by Kabat; see Al-lazikani et al. (1997) J. Molec. Biol. 273:927-948)). Other numbering approaches or schemes can also be used. As used herein, a CDR may refer to CDRs defined by either approach or by a combination of both approaches or by other desirable approaches. In addition, a new definition of highly conserved core, boundary and hyper-variable regions can be used.

The term “heavy chain” as used herein refers to the larger immunoglobulin subunit which associates, through its amino terminal region, with the immunoglobulin light chain. The heavy chain comprises a variable region (VH) and a constant region (CH). The constant region further comprises the CH1, hinge, CH2, and CH3 domains. In the case of IgE, IgM, and IgY, the heavy chain comprises a CH4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4). It is the nature of this chain that determines the “class” of the antibody as IgG, IgM, IgA IgG, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (VL) and a constant region (CL). Light chains are classified as either kappa or lambda (κ, λ). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

### III. ANTIBODIES AND MODIFICATIONS OF ANTIBODIES

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

In certain embodiments, are antibody conjugates. The conjugate can be, for example, a specific binding agent (such as an antibody) of the disclosure conjugated to other proteinaceous, carbohydrate, lipid, or mixed moiety molecule(s). Such antibody conjugates include, but are not limited to, modifications that include linking it to one or more polymers. In certain embodiments, an antibody is linked to one or more water-soluble polymers. In certain such embodiments, linkage to a water-soluble polymer reduces the likelihood that the antibody will precipitate in an aqueous environment, such as a physiological environment. In certain embodiments, a therapeutic antibody is linked to a water-soluble polymer. In certain embodiments, one skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In further embodiments, the conjugate can be, for example, a cytotoxic agent. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, calechaemicin, etc.), radioconjugates, enzyme conjugates (e.g., RNase conjugates, granzyme antibody-directed enzyme/prodrug therapy), and the like. Protein cytotoxins can be expressed as fusion proteins with the specific binding agent following ligation of a polynucleotide encoding the toxin to a polynucleotide encoding the binding agent. In still another alternative, the specific binding agent can be covalently modified to include the desired cytotoxin.

In additional embodiments, antibodies, or fragments thereof, can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin). The disclosure accordingly provides a molecule comprising an antibody molecule, wherein the molecule preferably further comprises a reporter group selected from the group consisting of a radiolabel, a fluorescent label, an enzyme, a substrate, a solid matrix, and a carrier. Such labels are well known to those of skill in the art, e.g., biotin labels are particularly contemplated. The use of such labels is well known to those of skill in the art and is described in, e.g., U.S. Pat. Nos. 3,817,837; 3,850,752; 3,996,345 and 4,277,437, each incorporated herein by reference. Other labels that will be useful include but are not limited to radioactive labels, fluorescent labels and chemiluminescent labels. U.S. Patents concerning use of such labels include for example U.S. Pat. Nos. 3,817,837; 3,850,752; 3,939,350 and 3,996,345. Any of the peptides of the present disclosure may comprise one, two, or more of any of these labels.

### A. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

### B. Humanized Antibodies and Production Thereof

Where the antibodies or their fragments are intended for therapeutic purposes, it may desirable to “humanize” them in order to attenuate any immune reaction. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al., J. Steroid Biochem., 26(1):83-92, 1987; Wood et al., J. Clin. Lab. Immunol., 17(4):167-171, 1985; and Shaw et al., J. Natl. Cancer Inst., 80(19):1553-1559, 1988; all of which references are incorporated herein by reference. “Humanized” antibodies can alternatively be produced by CDR or CEA substitution. Jones et al., Nature, 321:522-525, 1986 and Beidler et al., J. Immunol., 141(11):4053-4060, 1988, each of which is incorporated herein by reference. For this, human VH and VL sequences homologous to the VH and VL frameworks of the mouse monoclonal antibody can be identified by searching within the GenBank database. The human sequences with the highest homology are then was chosen as an acceptor for humanization. The CDR sequences of mouse monoclonal are then transferred to the corresponding positions of selected human frameworks.

### C. General Methods

It will be understood that monoclonal antibodies of the present disclosure have several applications. These include the production of diagnostic kits for use in detecting CD5L, as well as for treating diseases associated with increased levels of CD5L. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce antigen-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

Methods for generating hybrids of antibody-producing cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

In another aspect, there are provided monoclonal antibodies having clone-paired CDRs from the heavy and light chains as illustrated in Table 2. Such antibodies may be produced using methods described herein.

In another aspect, the antibodies may be defined by their variable sequence, which include additional “framework” regions. These are provided in Tables 3 and 4, which sequences encode or represent full variable regions. Furthermore, the antibody sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 4 and the amino acid sequences of Table 3.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### IV. METHODS OF TREATMENT

The present disclosure provides methods of treating a cancer patient with an anti-CD5L antibody as provided herein. Such treatment may also be in combination with another therapeutic regime, such as chemotherapy or immunotherapy. In some cases, the patient's cancer may overexpress CD5L. In some cases, the level of CD5L in the patient's serum may be elevated. In some cases, the patient's cancer may be bevacizumab resistant. In some cases, the patient's cancer may have recurred following bevacizumab treatment.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

Where clinical application of a therapeutic composition containing an antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 g/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An antibody may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an anti-CD5L antibody is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present disclosure to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### B. Immunotherapy

The skilled artisan will understand that additional immunotherapies may be used in combination or in conjunction with methods of the disclosure. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and/or a checkpoint inhibitor. In one aspect, the adoptive T cell therapy comprises autologous and/or allogenic T-cells. In another aspect, the autologous and/or allogenic T-cells are targeted against tumor antigens. Checkpoint inhibitors are discussed in greater detail above.

Immunomodulatory agents include immune checkpoint inhibitors, agonists of co-stimulatory molecules, and antagonists of immune inhibitory molecules. The immunomodulatory agents may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

Co-stimulatory molecules are ligands that interact with receptors on the surface of the immune cells, e.g., CD28, 4-1BB, OX40 (also known as CD134), ICOS, and GITR. As an example, the complete protein sequence of human OX40 has Genbank accession number NP_003318. In some embodiments, the immunomodulatory agent is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another co-stimulatory molecule that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Yet another co-stimulatory molecule that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immunomodulatory agent is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HLA-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Other immune inhibitory molecules that can be targeted for immunomodulation include STAT3 and indoleamine 2,3-dioxygenase (IDO). By way of example, the complete protein sequence of human IDO has Genbank accession number NP_002155. In some embodiments, the immunomodulatory agent is a small molecule IDO inhibitor. Exemplary small molecules include BMS-986205, epacadostat (INCB24360), and navoximod (GDC-0919).

### C. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present disclosure, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present disclosure to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present disclosure to improve the treatment efficacy.

### V. KITS

In various aspects of the embodiments, a kit is envisioned containing therapeutic agents and/or other therapeutic and delivery agents. In some embodiments, the present embodiments contemplates a kit for preparing and/or administering a therapy of the embodiments. The kit may comprise one or more sealed vials containing any of the pharmaceutical compositions of the present embodiments. The kit may include, for example, at least one anti-CD5L antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

| | | |
|---|---|---|
| REAGENT or | | |
| RESOURCE | SOURCE | IDENTIFIER |

Experimental Models: Cell Lines

| | | |
|---|---|---|
| SKOV3ip1 | MDA Cell Line Core | N/A |
| ID8 | Dr. Roby at UKansas | N/A |
| RF24 | ABM (Applied Biological | Catalog Number T0003 |
| | Materials) | |
| HEK293T | ATCC | CRL-3216 |

Experimental Models: Organisms/Strains

| | | |
|---|---|---|
| Mouse: Pparγ^{fl/fl}; Tie²⁻Cre^{+/-} | Dr Yihong Wan, University | N/A |
| female and male | of Texas Southwestern, (10 | |
| | mice/group) | |
| uMouse: Female athymic | Taconic (10 mice/group) | N/A |
| nude mice (NCr-nu) | | |

Chemicals, Peptides, and Recombinant Proteins

| | | |
|---|---|---|
| OCT | Miles, Inc., Elkhart, IN | Catalog Number 25608-930 |
| CD31 | Pharmingen, San Diego, CA | Catalog Number 557355 |
| Ki67 | Zymed, San Francisco, CA | Catalog number sc-23900 |
| RNeasy mini kit | Qiagen | Catalog Number 74104 |
| SYBR Green ER qPCR | Invitrogen, Carlsbad, CA | 4368708 |
| SuperMix Universal | | |
| SIRNAs | Sigma-Aldrich | N/A |
| Lipofectamine 2000 | Lipofectamine | Catalog Number 11668027 |
| Human recombinant CD5L | Sino Biological, Beijing, | Catalog Number 10791- |
| protein | China | H08H |
| Bevacizumab | Genetech | N/A |
| MTT reagent | Sigma-Aldrich | M2128 |
| His-tagged VEGF-A | Abnova, Taiwan | Catalog Number P5816 |
| His-tagged CD19 | Life Technologies, Carlsbad, | Catalog Number |
| | CA | 11880H08H250 |
| 6-His Peptide tagged | BioLegend, San Diego, CA | Catalog Number 931601 |
| DNA library | TriLink Biotechnologies, San | Fitzwater T. and Polisky B. |
| | Diego, CA | (1996) |
| M-MuLV Reverse | Roche, Indianapolis, IN | Catalog Number |
| Transcriptase | | 11062603001 |
| Ni²⁺ NTA Magnetic Agarose | Qiagen, Hilden, Germany | Catalog Number 36111 |
| Beads | | |
| TRIzol Reagent | Life Technologies, Carlsbad, | Catalog Number 15596026 |
| | CA | |
| TOPO Cloning | Life Technologies, Carlsbad, | Catalog Number K4500- |
| | CA | 01SC, K4550-01SC, K4600- |
| | | 01SC |

Antibodies: Expressed in HEK293 suspension cells and purified
using protein A affinity resin to purity >95%.

| | | |
|---|---|---|
| AKT | Cell Signaling Technologies | Catalog Number 4691/9272 |
| Phospho-AKT | Cell Signaling Technologies | Catalog Number 4060 |
| PPARγ | Cell Signaling Technologies | Catalog Number 2430 |
| Anti-human CD5L | Santacruz Biotechnology | Catalog Number sc-514281 |
| | R&D Systems | Catalog Number AF2797 |
| β-actin | Sigma-Aldrich | A5441 |
| Vinculin | Sigma-Aldrich | V9131 |
| Anti-Rabbit secondary | Sigma-Aldrich | NA934 |
| antibodies conjugated with | | |
| horseradish peroxidase. | | |
| Anti-Mouse secondary | Sigma-Aldrich | NA931 |
| antibodies conjugated with | | |
| horseradish peroxidase. | | |
| pLenti-C-mGFP-human | Origene | RC206528L2 |
| CD5L vector | | |

Critical Commercial Assays

| | | |
|---|---|---|
| Click-iT EdU Assay Kit | Invitrogen | Catalogue number C10632 |
| CD5L ELISA Kit | Mybiosource, San Diego, | Catalogue Number |
| | CA, USA | MBS2024653 |
| Verso cDNA kit | Thermo Scientific | Catalogue number AB1453B |
| RNeasy mini kit | Qiagen | Catalogue number 74106 |
| EZ ChIPTM kit | Millipore, Temecula, CA | Catalogue number 17-371 |

Software and Algorithms

| | | |
|---|---|---|
| GraphPad Prism 7.0. | SPSS version 12 for | SPSS version 12 for |
| | Windows statistical software | Windows statistical software |
| Windows statistical software | SPSS version 12 for | SPSS, Inc., Chicago, IL |
| | Windows statistical software | |
| Kaplan-Meier survival | SPSS version 12 for | SPSS version 12 for |
| curves | Windows statistical software | Windows statistical software |
| ClustalW2 software | N/A | N/A |
| Tree VieX program | N/A | N/A |
| Monolith NT.115 | NanoTemper Technologies | Stoltenburg R et al. PLOS |
| | GmbH, Munchen, Germany | One. 2015 |

Cell Lines and Culture. Human epithelial ovarian cancer cell line SKOV3ip1 and mouse ovarian cancer cell line ID8 were grown as previously described (Pradeep et al., 2015). Human immortalized umbilical endothelial cells (RF24) were grown in MEM medium containing supplements (non-essential amino acids, sodium pyruvate, MEM vitamins, and glutamine; Life Technologies, Grand Island, NY). Cell culture was performed at 37° C. in a 5% CO₂ incubator with 95% humidity. For in vivo injections, cells were first washed with PBS twice, followed by trypsinization and centrifugation at 1,200 rpm for 5 min at 4° C. Cells were then reconstituted in serum-free Hank's balanced salt solution (Life Technologies). Only single-cell suspensions with >95% viability were used for in vivo experiments (as determined by trypan blue exclusion).

Quantitative Real-Time Reverse-Transcriptase PCR Validation. Quantitative real-time reverse-transcriptase PCR was carried out using 50 ng of total RNA isolated from cells using the RNeasy mini kit (Qiagen). Complementary DNA (cDNA) was synthesized from 0.5-1.0 μg of total RNA by using a Verso cDNA kit (Thermo Scientific). Quantitative PCR (qPCR) analysis was performed in triplicate by using the reported primers (Table 1) and the SYBR Green ER qPCR SuperMix Universal (Invitrogen, Carlsbad, CA) with the Bio-Rad Thermocycler (Bio-Rad Laboratories, Hercules, CA). Quantification was performed using the 2^{-ΔΔCT} method normalizing to control for percent fold changes (Donninger et al., 2004).

TABLE 1
Quantitative-PCR primer lists

| No. | Primers | Sequence(5'-3') |
|---|---|---|
| 1 | Human CD5L F | 5'-CTGCTTGTTCTCCTGAGCCC-3' |
| | | (SEQ ID NO: 344) |
| 2 | Human CD5L R | 5'-TCAAAGGGTCAGGGTTGAGC-3' |
| | | (SEQ ID NO: 345) |
| 3 | Human PPAR-γ F | 5'-GCCCTTTGGTGACTTTATGGA-3' |
| | | (SEQ ID NO: 346) |
| 4 | Human PPAR-γ R | 5'-GCAGCAGGTTGTCTTGGATG-3' |
| | | (SEQ ID NO: 347) |
| 5 | Human CD36 F | 5'-GAGAACTGTTATGGGGCTAT-3' |
| | | (SEQ ID NO: 348) |
| 6 | Human CD36 R | 5'-TTCAACTGGAGAGGCAAAGG-3' |
| | | (SEQ ID NO: 349) |
| 7 | Tie2 Cre | 5'-CGCATAACCAGTGAAACAGCATTGC-3' |
| | transgene F | (SEQ ID NO: 350) |
| 8 | Tie2 Cre | 5'-CCCTGTGCTCAGACAGAAATGAGA-3' |
| | transgene R | (SEQ ID NO: 351) |

siRNA Constructs and Delivery. siRNAs were ordered from Sigma-Aldrich (Woodlands, TX). Our siRNA control consisted of a non-silencing siRNA that did not share sequence homology with any known human mRNA based on a BLAST search. In vitro transient transfection was performed as previously described (Landen et al., 2005). In brief, siRNA (4 j g) was incubated with 10 μL of Lipofectamine 2000 transfection reagent (Lipofectamine) for 20 minutes at room temperature followed by the addition to cells cultured in 10 cm plates at 80% confluence.

Reverse Phase Protein Array (RPPA) and Western Blot Analysis. RF24 cells were cultured in the presence or absence of human recombinant CD5L protein (Sino Biological, Beijing, China). Corresponding cell lysate was then submitted for RPPA analysis. Western blot analysis was performed as previously reported (Pradeep et al., 2015, Haemmerle et al., 2017). Cell lysate of RF24 cells was collected after treatment with human recombinant CD5L protein and activation of AKT signaling was performed using anti-human CD5L, p-AKT, AKT, and PPAR-γ antibodies, followed by appropriate secondary antibodies conjugated with horseradish peroxidase. Experiments were done in duplicate and repeated at least twice.

Cell Proliferation Assay. Cell proliferation experiments were performed using the Click-iT EdU Assay Kit (Invitrogen). Cells were seeded into 6-well plates and cultured in a phenol-red free Opti-MEM for 48 hours. Cells were then harvested for the assessment of proliferation after CD5L protein or siRNA CD5L treatment.

Enzyme-Linked Immunosorbent Assay. CD5L expression levels were determined using a CD5L ELISA Kit (Mybiosource, San Diego, CA, USA) following the manufacturer's recommendations for both cell culture media as well as human blood samples.

Lentivirus-Mediated CD5L Overexpression. The pLenti-C-mGFP-human CD5L vector (RC206528L2) was purchased form Origene (Rockville, MD). HEK293T cells were co-transfected with the pLenti-C-mGFP-human CD5L vector and packaging plasmids. After 48 hours, supernatant containing infectious viral particles was collected, filtered using 0.45 μm filters, and stored in aliquots at −80° C. To generate cells that ectopically overexpressed CD5L, RF24 cells were incubated for 48 hours with viral particles. Cells were then washed with PBS and further incubated in culture medium. After 48 hours, GFP positive cells were collected using the FACS Aria II sorter.

Induction of Bevacizumab-Resistance in RF24 Cells. Bevacizumab-resistant cell line were derived from original parental RF24 cell line by continuous exposure to bevacizumab. RF24 cells were treated with bevacizumab (1 mg/ml, IC₅₀) for 72 h. This media was then removed and the cells were allowed to recover for 7 days. Cells were continuously maintained in the presence of bevacizumab at IC₅₀ concentrations.

Drug Sensitivity Assay (MTT). Cells (5×10³) were seeded in 96-well plates and allowed to adhere overnight at 37° C. Briefly, following treatment of cells with Bevacizumab for 72 h, MTT reagent [3-(4,5-Dimethylthiazol-2-yl)-2,5-diphenyltetrazoliumbromide] was added to each well and incubated for 4 h at 37° C. Dimethylsulphoxide (DMSO) was added to each well and mixed for 5 min on an orbital shaker. Absorbance was recorded at 450 nm and sensitivity to bevacizumab was calculated based on cell proliferation measurements at 72 h.

Cell Migration Assay. Migration of RF24 cells was examined in the presence or absence of hCD5L siRNA or anti-CD5L antibody using Transwell 0.4 μm pore polycarbonate membrane inserts coated with 0.1% gelatin. After post-transfection of 48 hours with hCD5L siRNAs or after treatment with anti-CD5L antibody (40 μg/ml), RF24 cells (1.0×10⁵) in MEM serum-free medium were seeded into the upper chamber of the Transwell 0.4 μm pore polycarbonate membrane insert (Corning, Lowell, MA). CD5L protein (400 ng/ml) was also added to the cell suspension prior to seeding, where indicated above. The insert was then placed in a 24-well plate containing MEM medium with 15% serum in the lower chamber as chemoattractant. After allowing cells to migrate for 6 hours in a humidified chamber, those that had migrated were stained with hematoxylin and counted by light microscopy in five random fields (×200 original magnification) per sample. Experiments were done in duplicate and repeated three times.

Tube Formation Assay. Matrigel (12.5 mg/mL) was thawed at 4° C., and either 50 μL was quickly added to each well of a 96-well plate or 10 μL to each well of a 15-well plate and then allowed to solidify for 30 min at 37° C. The wells were then incubated for 6 h at 37° C. with RF24 cells (5,000-6000 per well), which had previously been treated with either CD5L siRNA (for 48 h), PI3kinase inhibitor or AKT inhibitor (for 6 h), or anti-CD5L antibody (40 μg/ml, added at time of cell seeding). Where indicated above, CD5L protein (400 ng/ml) was also added at time of cell seeding. Experiments were performed in triplicate and repeated at least twice. Using an Olympus IX81 inverted microscope, five images per well were taken at ×100 magnification. The number of nodes (defined as at least three cells that formed a single point) and tubes (defined as a non-segmented circle formed from endothelial cells) per image was quantified. The highest and lowest values were removed from each group to account for cell clumping.

Promoter Analysis and Chromatin Immunoprecipitation (ChIP) Assay. RF24 cells were cultured in hypoxia condition for 16 h. After hypoxic culture, ChIP assays were performed by using EZ ChIP™ kit (Millipore, Temecula, CA) as described by the manufacturer. In brief, cross-linked cells were collected, lysed, sonicated, and subsequently subjected to immunoprecipitation with PPAR-γ (Cell signaling) antibody or IgG control. Immunocomplexes were collected with protein G agarose beads and eluted. Cross-links were reversed by incubating at 65° C. DNA was then extracted and purified for subsequent PCR amplification using gene-specific primers (Table 1).

Orthotopic in vivo Model of Ovarian Cancer. Female athymic nude mice (NCr-nu) were purchased from the NCI-Frederick Cancer Research and Development Center (Frederick, MD) and maintained as described previously (Landen et al., 2005). All mouse studies were approved by the Institutional Animal Care and Use Committee. Mice were cared for in accordance with guidelines set forth by the American Association for Accreditation of Laboratory Animal Care and the US Public Health Service Policy on Humane Care and Use of Laboratory Animals. For tumor cells injection, SKOV3ip1 cells (1×10⁶) were injected intraperitoneally. For the anti-CD5L antibody experiments, control or experimental antibody was injected intraperitoneally once weekly at a dose of 10 mg/kg body weight. After mice were euthanized with CO₂, their tumor weight and number and distribution of tumor nodules was recorded. Individuals who performed the necropsies were blinded to the treatment group assignments. Tissue specimens were either fixed using 10% buffered formalin, OCT (Miles, Inc., Elkhart, IN) or snap-frozen in liquid nitrogen.

Novel Anti-CD5L Antibody Generation. Monoclonal antibodies were screened and selected from two antibody sources. One source is from CD5L immunized rabbit single B cells. Briefly, two New Zealand white rabbits were immunized with 0.5 mg recombinantly expressed human CD5L protein (Sino Biological). After the initial immunization, animals were given 3 boosters in a three-week interval until serum titers reached to 10⁶. Single B cells were isolated and screened for CD5L binding antibodies using ELISA for initial positive hits. A human naïve scFv phage display library (10¹⁰ diversity) made in Drs. Z. An and N. Zhang joint laboratory was also used as another source for selection of monoclonal antibodies against CD5L. Solid phase panning method was used for selection of CD5L binders by ELISA (Bu et al., 2019). Antibody variable coding regions in the positive hits were cloned and sequenced using a method reported previously (John et al., 2018). Full length antibody heavy and light chain constructs were made with human IgG constant region sequences in fusion with cloned variable sequences for expression of monoclonal antibodies in human embryonic kidney freestyle 293 (HEK293F) cells (Life science Technologies). Antibodies were purified using protein A/G affinity resin to purity >95% using a method as we described previously (Noh et al., 2017).

Immunohistochemical and Immunofluorescence Staining of Xenografts. Immunohistochemical analyses for cell proliferation (Ki67, 1:200, Zymed, San Francisco, CA) and MVD (CD31, 1:500, Pharmingen, San Diego, CA) were performed as described previously (Thaker et al., 2006, Lu et al., 2010). For statistical analyses, sections from 5 randomly selected tumors per group were stained, and 5 random fields per tumor were scored. Pictures were taken at ×200 or ×100 magnification. To quantify MVD in the mouse tumor samples, the number of blood vessels staining positive for CD31 was recorded in 10 random 0.159-mm² fields at ×200 magnification. To quantify Ki67 expression, the number of positive cells was counted in 10 random 0.159-mm² fields at ×100 magnification (Thaker et al., 2006, Lu et al., 2010). All staining was quantified by two investigators in a blinded fashion. Analysis of CD5L expression in tumor endothelial cells from our ovarian cancer cohort was scored as either 1 (negative), 2 (low), or 3 (high).

Tie2-cre:PPAR-γ Knockout Mice. Pparg^{fl/fl}:Tie2-Cre^{+/-} female and male mice were generously provided by Dr Yihong Wan, (University of Texas Southwestern). To selectively delete PPAR-γ in endothelial cells, females were crossed with males to obtain the PPAR-γ conditional knock-out mice. Genomic DNA was isolated from tail biopsies of the mouse. Tie2-cre transgene and floxed PPAR-γ allele were distinguished by PCR using primers (Table 1). For tumor cells injection, ID8 cells (1×10⁶) were injected intraperitoneally in Tie2-cre:PPAR-γ KO mice. After mice were euthanized with CO₂, their tumor weight and number and distribution of tumor nodules was recorded. Individuals who performed the necropsies were blinded to the treatment group assignments. Tissue specimens were either fixed using either 10% buffered formalin, OCT (Miles, Inc., Elkhart, IN) or snap-frozen in liquid nitrogen. For B20 therapy experiments, B20 was give twice weekly at a dose of 5 mg/kg body weight after 7 days from ID8 tumor injection. Depending on the experimental design, B20 treatment continued until either the planned endpoint was reached, or until mice became moribund or deceased.

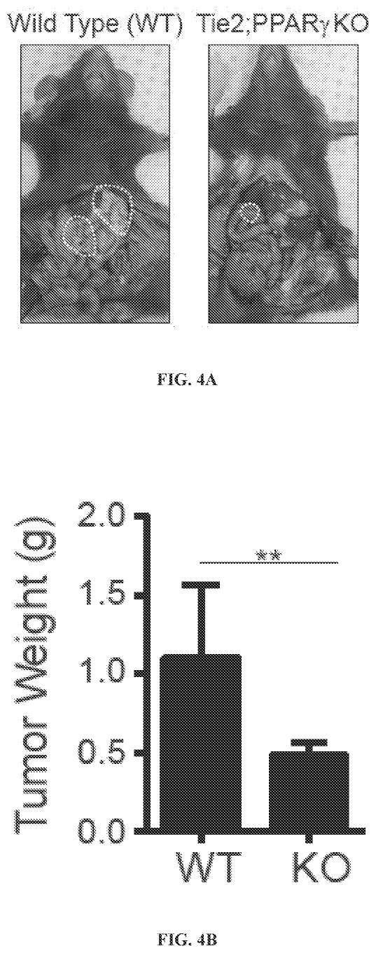

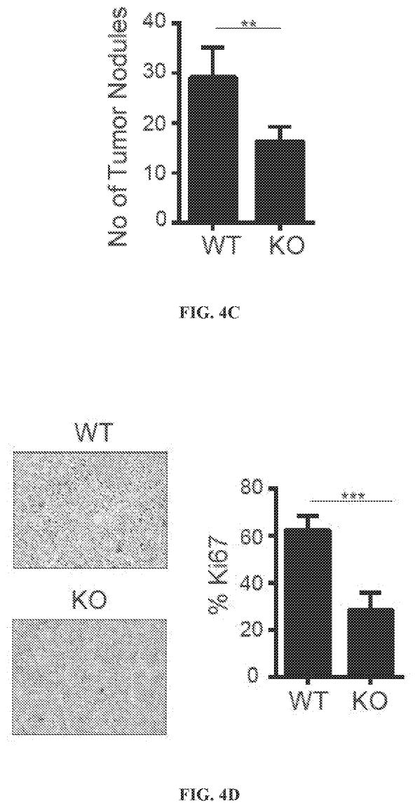

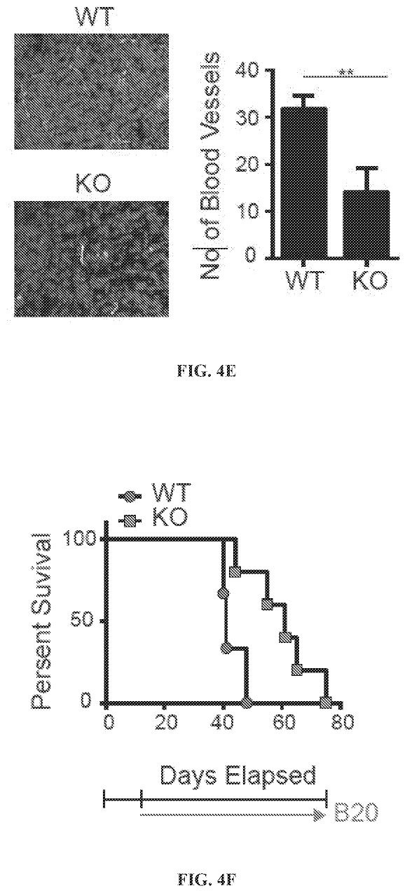

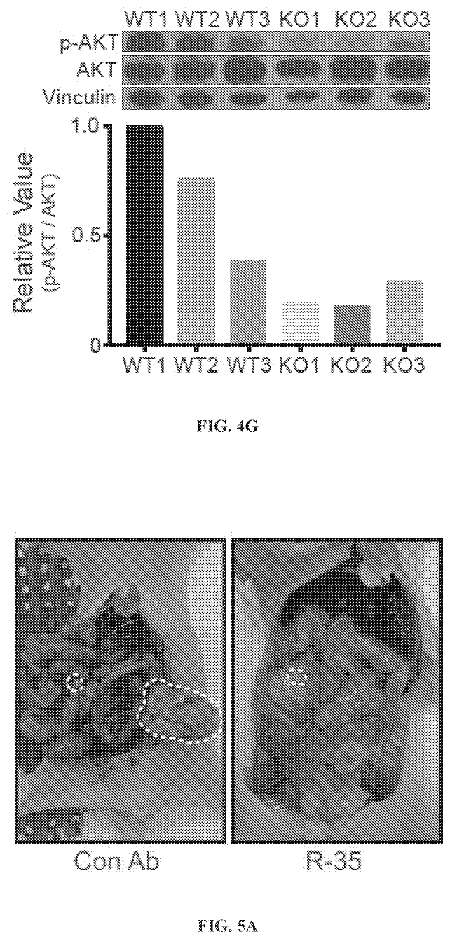

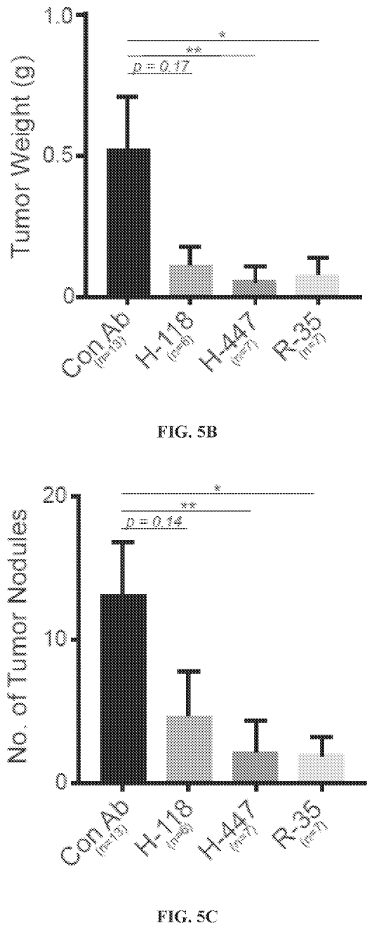

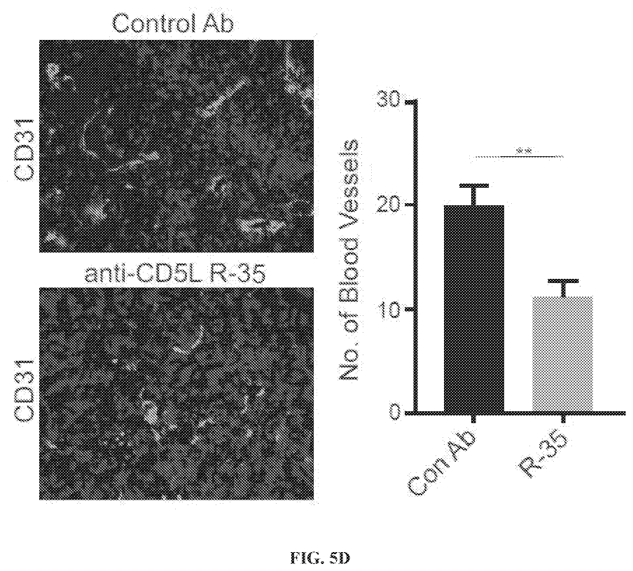

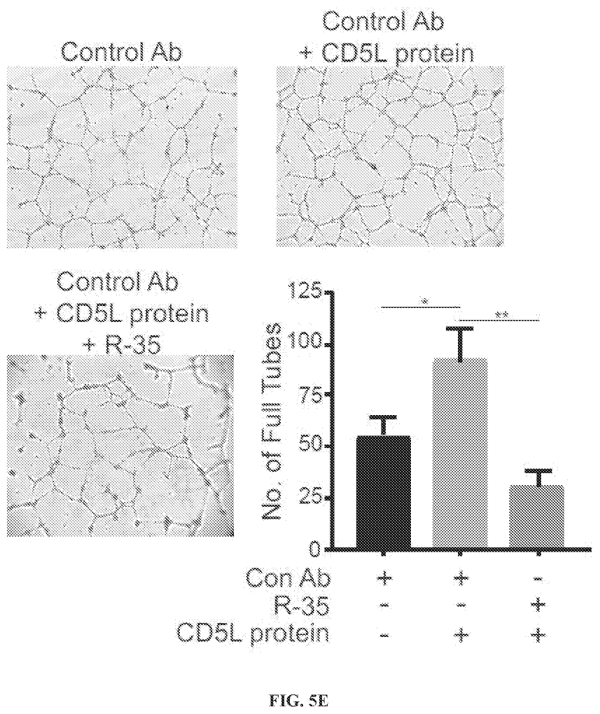

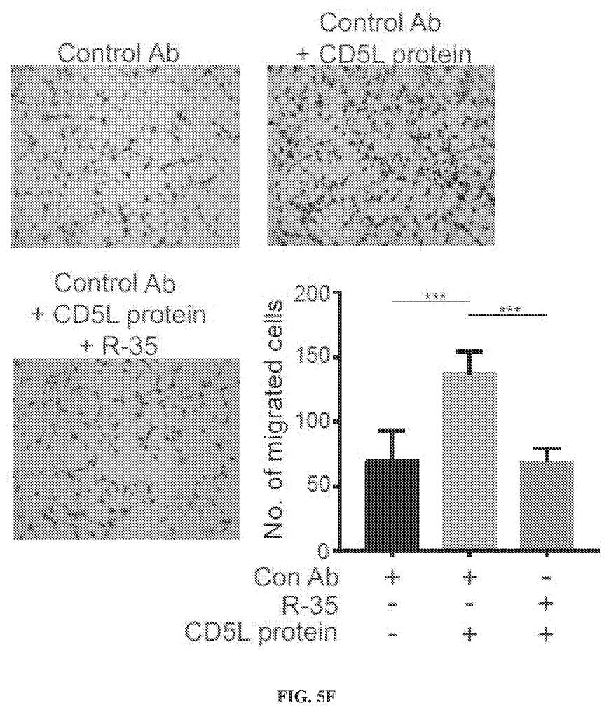

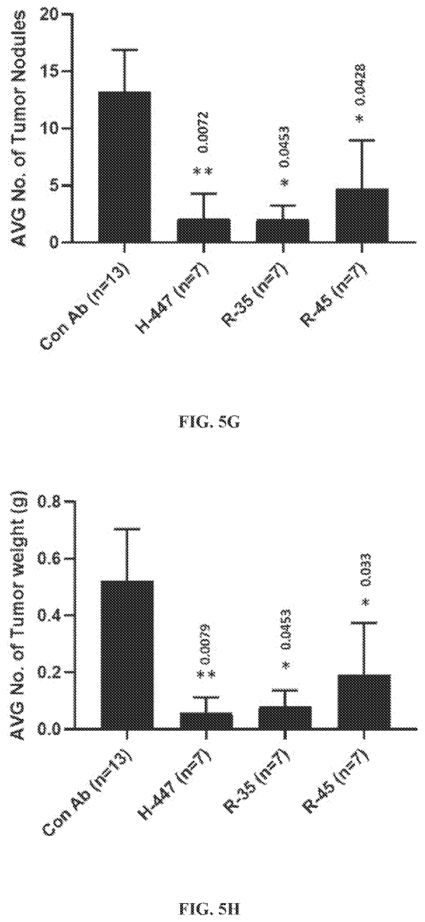

Statistical Analyses. Kaplan-Meier survival curves were generated and compared with the use of a log-rank statistic to assess the effect of tumor vascular CD5L expression on human overall survival, as well as to determine survival in our Tie2-cre; PPAR-γ KO mice model. For the animal experiments in FIGS. 4A-4G mice were assigned per treatment group. This sample size gave 80% power to detect a 50% reduction in tumor weight with a 95% confidence interval. As the in vivo experiment in FIG. 5 was designed to screen antibody candidates, only 7 mice were assigned per treatment group. Tumor weights and the number of tumor nodules for each group were compared using either the Student's t-test (for comparisons of two groups) if the distribution was normal, or Mann-Whitney if the distribution was non-normal. A P-value of less than 0.05 were deemed statistically significant. All statistical tests were two-sided and were performed using either SPSS version 12 for Windows statistical software (SPSS, Inc., Chicago, IL) or GraphPad Prism 7 for Windows (GraphPad Software, La Jolla, CA).

### Example 2—Adaptive Genomic Changes in Tumor Endothelial Cells

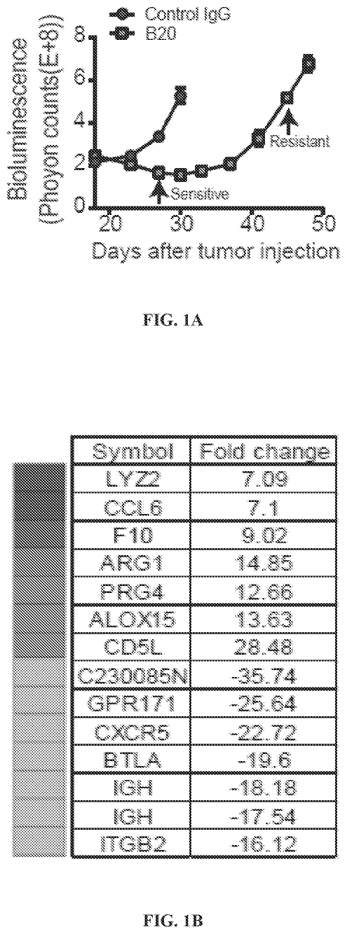

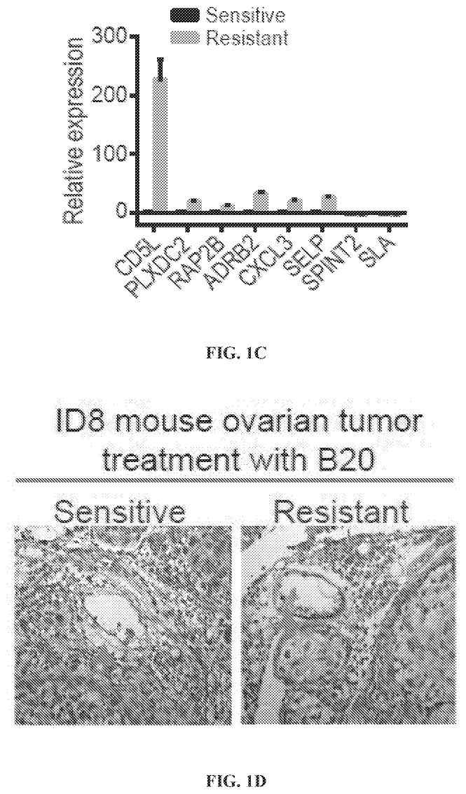

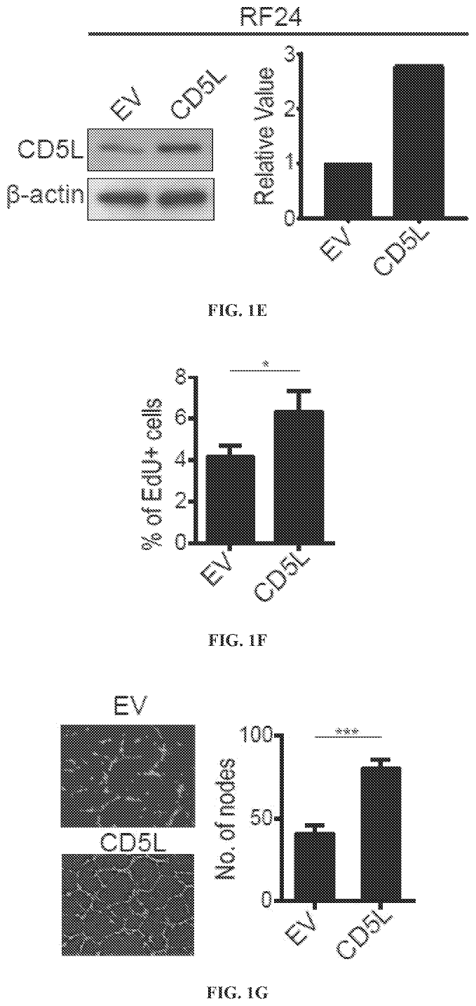

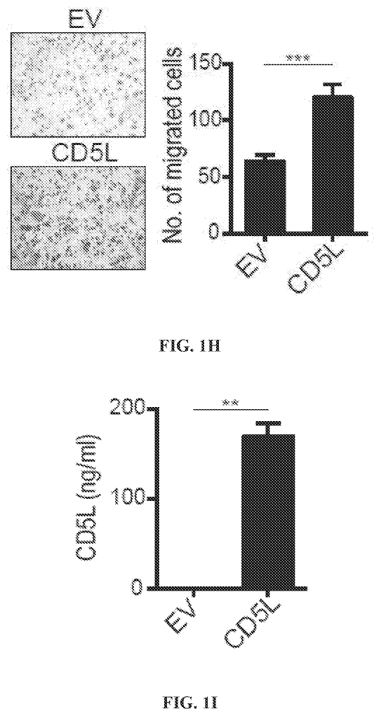

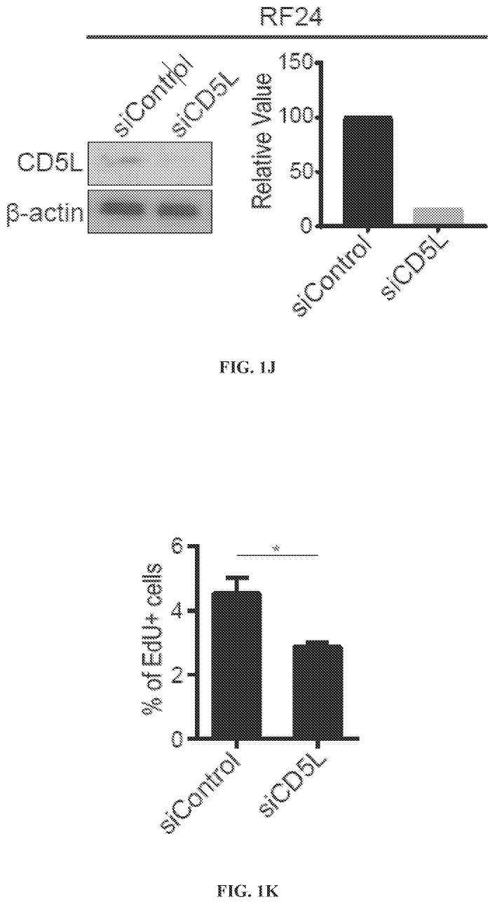

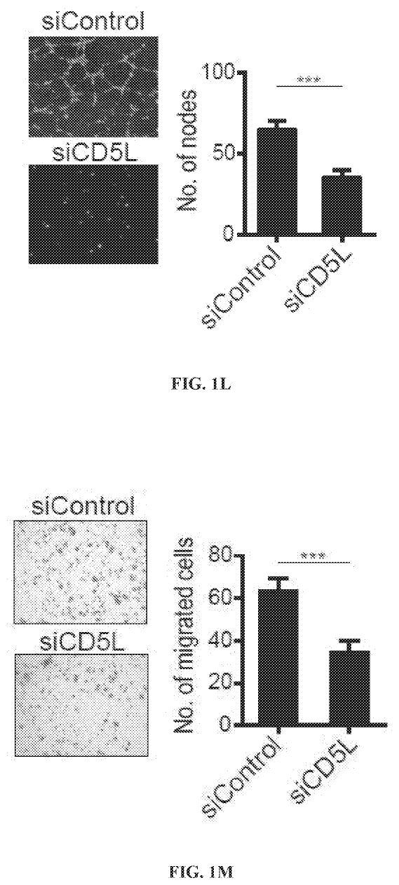

To identify possible targets involved in adaptive resistance, the syngeneic ID8 ovarian cancer mouse model was used. Mice were treated with the B20 antibody and tumors were obtained at time points demonstrating both sensitivity and resistance (FIG. 1A). Endothelial cells were then isolated from sensitive and resistant tumor samples and gene expression profiling was performed using isolated mRNA. A large number of genes displayed differential expression between the endothelial cells from sensitive versus resistant tumors, with CD5L demonstrating the largest difference of 28.48 fold higher in the resistant endothelial cells (FIG. 1B). A subset of genes was then selected from the array and subjected to qRT-PCR to confirm the observed increase in CD5L expression in the resistant setting (FIG. 1C). Using immunohistochemistry, CD5L protein expression was found to be significantly higher in the endothelial cells from resistant tumors compared to those from sensitive tumors (FIG. 1D).

Next, the biological effects of CD5L upregulation in tumor endothelial cells was examined. To determine the function of CD5L in tumor angiogenesis, CD5L overexpressing RF24 cells were generated (FIG. 1E). These cells displayed elevated proliferation, increased tube formation capacity, and increased cell migration compared to controls (FIGS. 1F and 1H). Consistent with CD5L being a primarily secreted protein, the concentration of CD5L in the media from RF24 cells overexpressing CD5L was shown to be significantly higher than media from control RF24 cells (empty vector) (FIG. 1I). To confirm that the overexpression of CD5L was the primary source of these observed effects, control RF24 cells were treated with CD5L siRNA. More than a 90% knockdown of CD5L protein levels was seen within 72 hours compared with a non-targeting siRNA (FIG. 1J). Notably, cells treated with CD5L siRNA showed significantly reduced proliferation, tube formation capacity, and cell migration compared to cells treated with control siRNA (FIGS. 1K and 1M).

### Example 2—CD5L is Upregulated Through Hypoxia-Induced PPAR-γ Overexpression

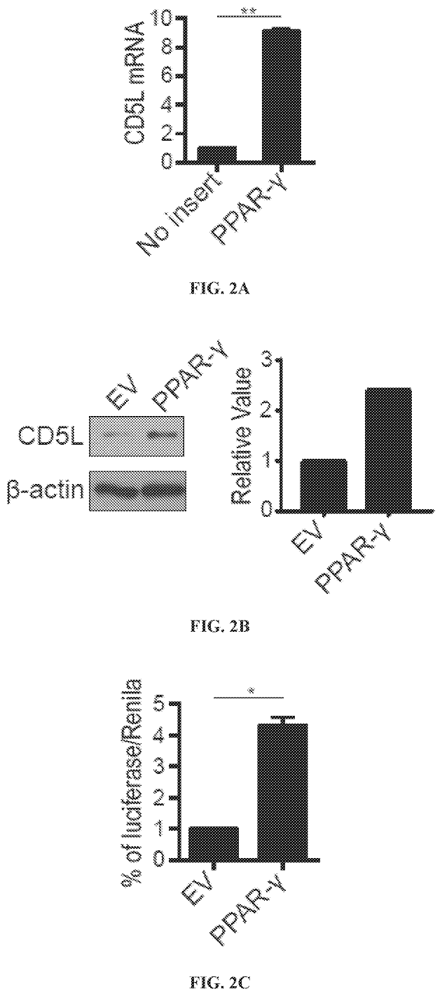

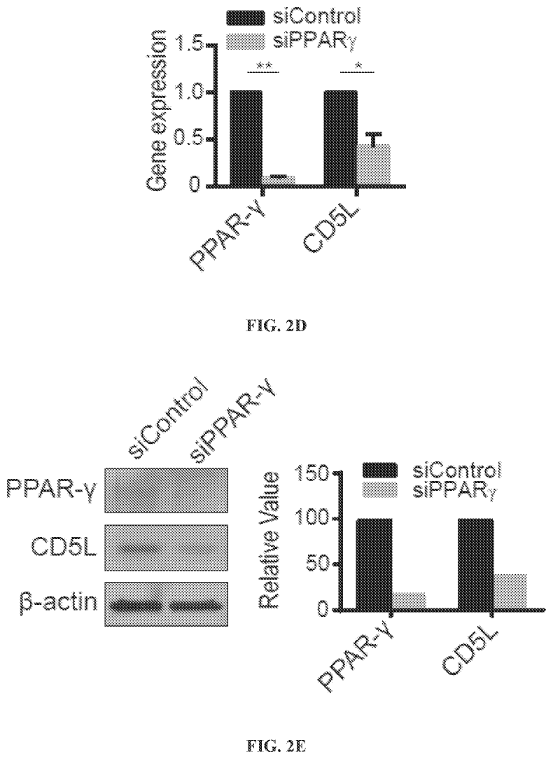

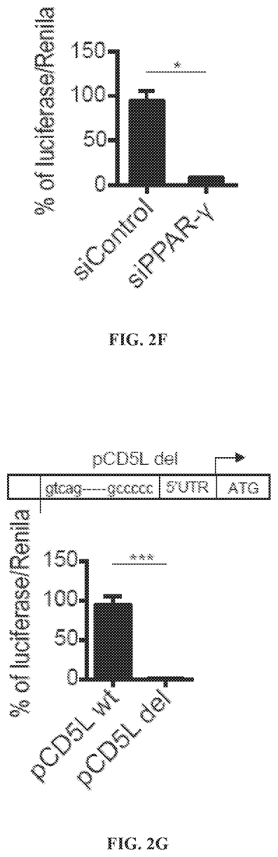

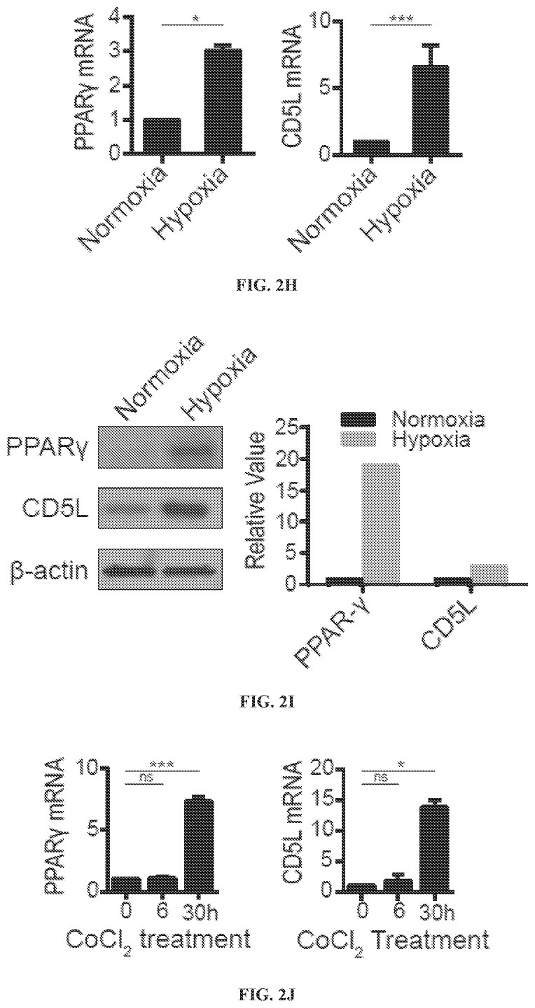

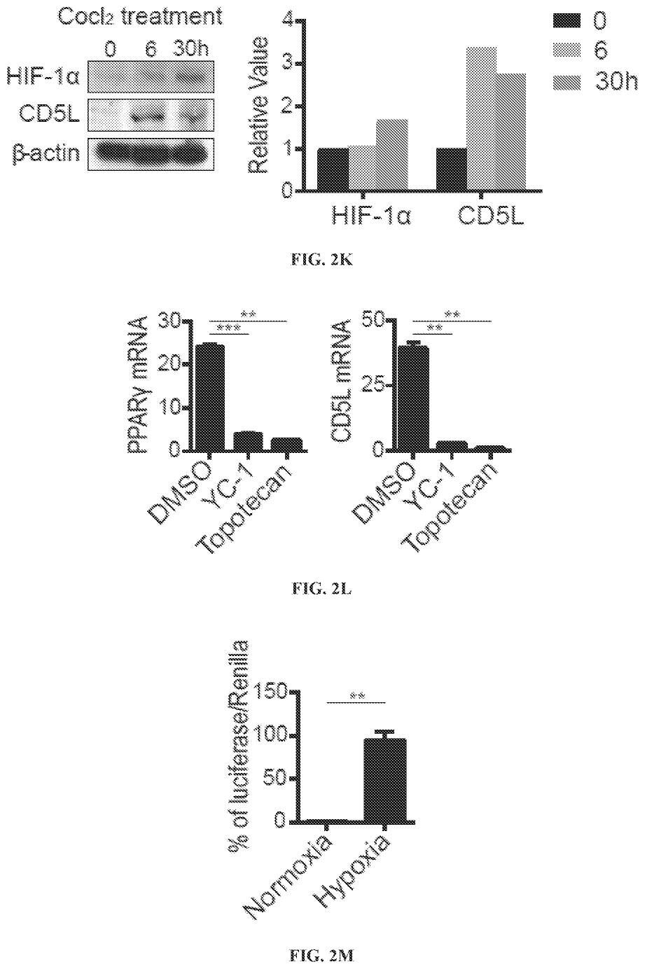

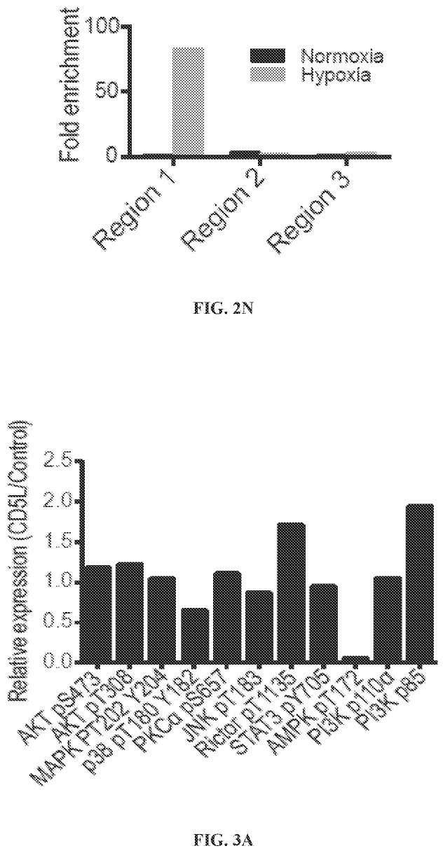

To determine possible mechanisms of CD5L elevation in tumor endothelial cells, regulation of CD5L gene transcription was examined. Upon analysis of the CD5L promoter sequence, a putative binding site (shown in red) for the transcription factor PPAR-γ was identified (FIG. 8). To test whether PPAR-γ may serve as an upstream regulator of CD5L, PPAR-γ was ectopically expressed in RF24 endothelial cells. Compared with controls, endothelial cells with elevated PPAR-γ demonstrated a significant increase in both CD5L mRNA and protein (FIGS. 2A and 2B). Additionally, a CD5L promoter construct (pCD5L WT) was generated and a significant increase in luciferase activity was found when transfected into PPAR-γ overexpressing RF24 cells compared to wild type RF24 cells (FIG. 2C). To further prove that PPAR-γ expression was responsible for the observed increases in CD5L expression and promoter activity, wild-type RF24 cells were treated with PPAR-γ siRNA. Cells that were treated with PPAR-γ siRNA showed a significant reduction in both CD5L mRNA and protein (FIGS. 2D and 2E), as well as decreased luciferase activity from the CD5L promoter construct (FIG. 2F). Next, the PPAR-γ binding site was deleted from the CD5L promoter construct (pCD5L del) to determine whether the CD5L increase observed was due to PPAR-γ specific binding. After co-transfecting RF24 cells with the PPAR-γ expression plasmid and either pCD5L WT or pCD5L del, it was found that the mutated PPAR-γ binding site resulted in significantly reduced luciferase activity compared to the non-mutated promoter, indicating that PPAR-γ directly regulates CD5L expression (FIG. 2G).

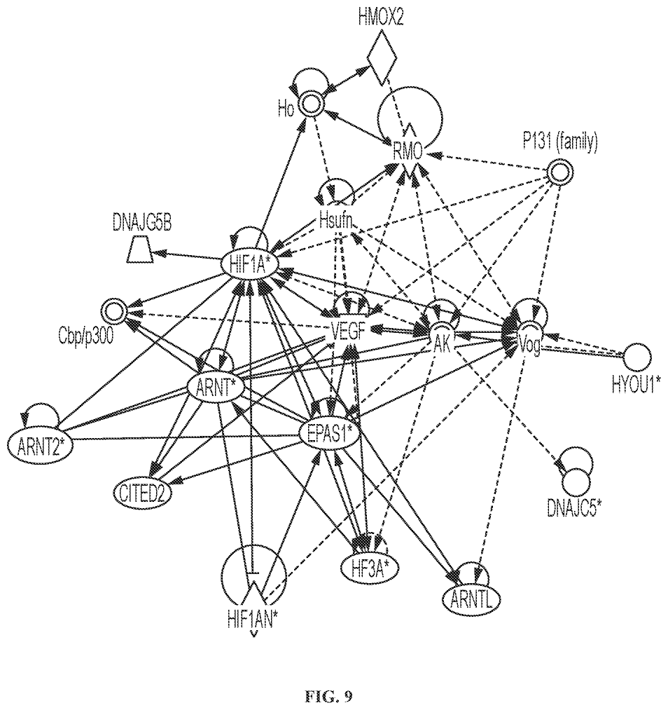

Next, whether any other factors upstream of PPAR-γ played a role in the upregulation of CD5L was sought to be determined. Using the initial gene expression dataset generated from anti-VEGF resistant endothelial cells, an ingenuity pathway analysis (IPA) was performed and a close correlation with hypoxia signaling proteins (HIF1A, EPAS1, ARNT) was found (FIG. 9). To confirm this relationship, WT RF24 cells were grown under hypoxic and normoxic conditions and a significant increase in both PPAR-γ and CD5L mRNA and protein levels was found in cells grown under hypoxic compared to normoxic conditions (FIGS. 2H and 2I). Extending this finding further, RF24 cells were incubated with a HIF1A stabilizing compound (cobalt chloride—CoCl₂) for 6 and 30 hours under normoxic conditions. Both PPAR-γ and CD5L mRNA expression was significantly higher after 30 hours of CoCl₂ incubation compared to 6 hours as well as no treatment, further demonstrating that hypoxia-like conditions lead to the upregulation of PPAR-γ and CD5L (FIG. 2J). In addition, HIF1A and CD5L protein expression were also increased at longer incubation times of CoCl₂, as expected (FIG. 2K). To determine whether HIF1A blockade resulted in the opposite effect on PPAR-γ and CD5L, two known HIF1A inhibitors, YC-1 and topotecan, were employed. After treatment of WT RF24 cells with either YC-1 or topotecan, a significant decrease in both PPAR-γ and CD5L mRNA expression was found compared to DMSO control (FIG. 2L). Furthermore, CD5L promoter activity (pCD5L WT) was significantly increased when exposed to hypoxic versus normoxic conditions (FIG. 2M). Lastly, chromatin immunoprecipitation (ChIP) analysis of the CD5L promoter was performed using an anti-PPAR-γ antibody under hypoxic and normoxic conditions. The PPAR-γ binding site (located in region 1 of the CD5L promoter) had a significantly higher fold-enrichment for PPAR-γ under hypoxic compared to normoxic conditions (FIG. 2N). Moreover, the selectivity of PPAR-γ for the specific promoter sequence in region 1 was identified, as there was only minimal binding in regions 2 or 3 under normoxic or hypoxic conditions.

### Example 3—Exogenous CD5L Increases PI3K/AKT Signaling in Endothelial Cells

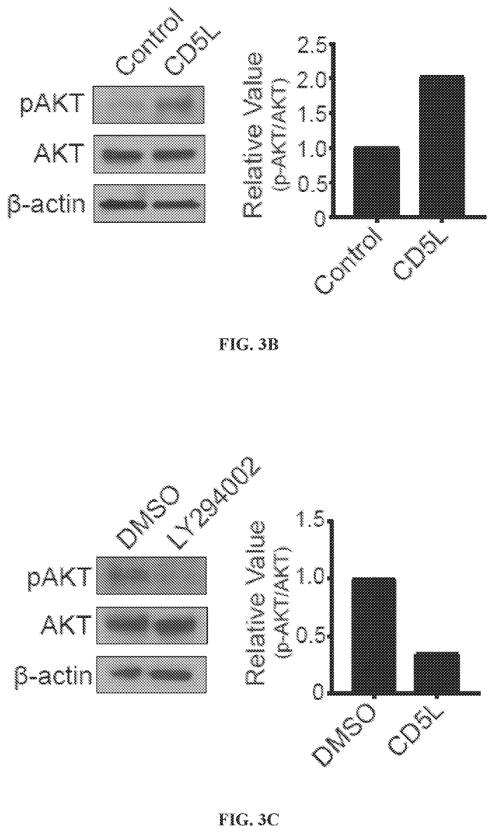

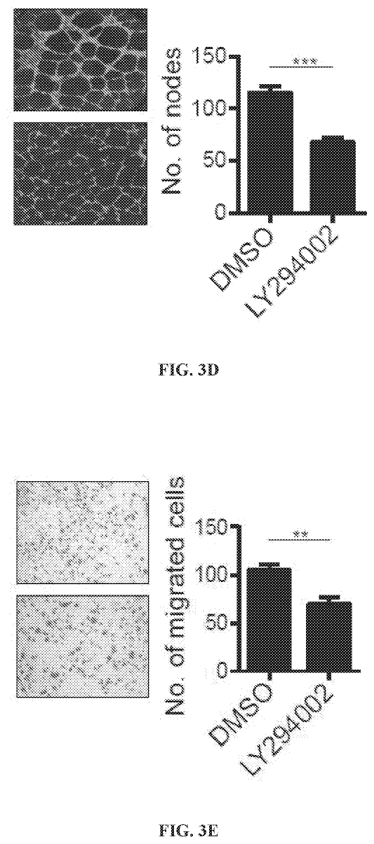

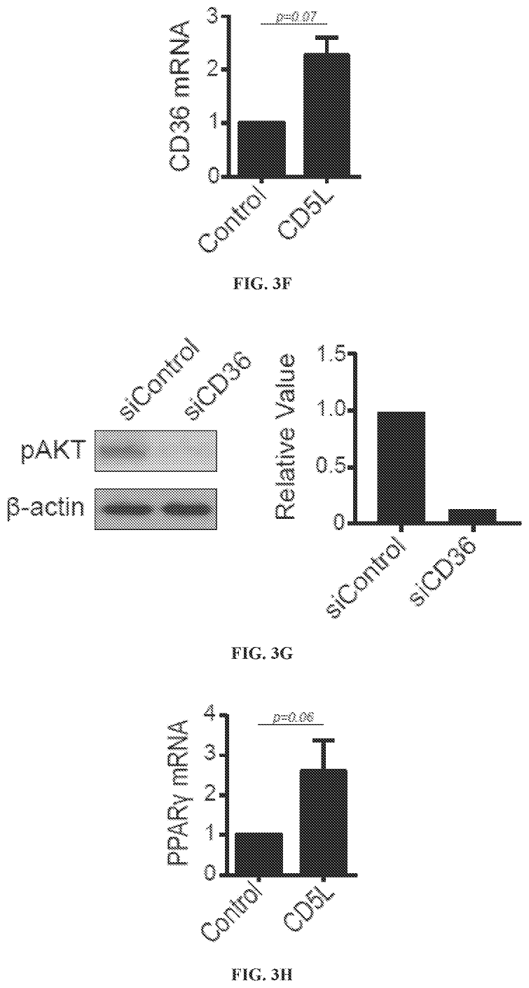

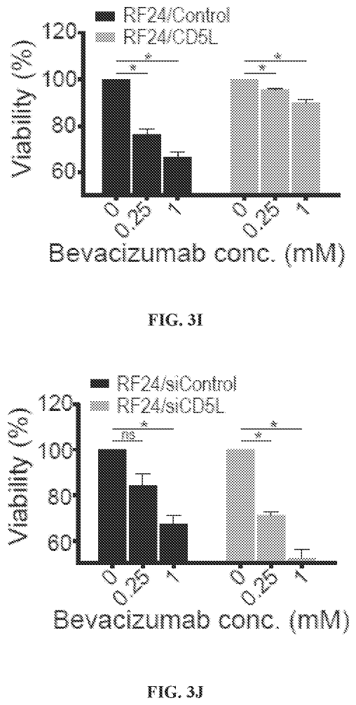

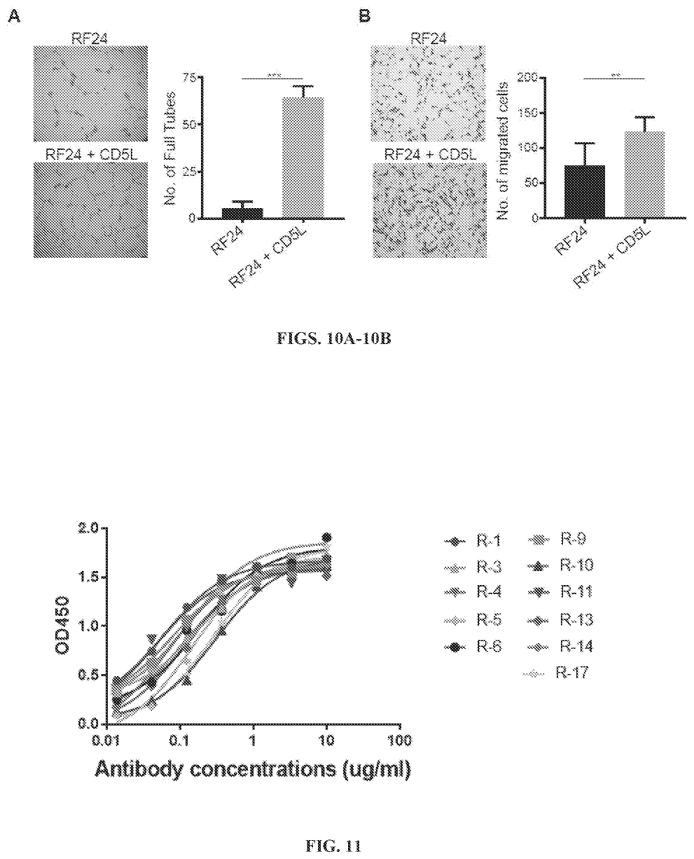

As CD5L is primarily a secreted protein shown to act in a paracrine fashion, RF24 endothelial cells were exogenously treated with CD5L to determine the downstream signaling effects. Reverse phase protein array (RPPA) analyses of both CD5L-treated and untreated RF24 endothelial cells were performed, and the CD5L-treated cells showed activation of PI3K/AKT signaling (FIG. 3A). To validate these results, the protein levels of AKT and phosho-AKT in CD5L-treated RF24 cells were measured compared with untreated controls. The RF24 cells, which were pre-treated with CD5L, had increased expression of p-AKT compared with the untreated cells (FIG. 3B). Furthermore, addition of the PI3K inhibitor LY294002 after CD5L pre-treatment mitigated these stimulating effects on p-AKT (FIG. 3C). Similarly, both tube formation and cell migration of RF24 cells increased significantly after CD5L treatment (FIGS. 10A-10B); however, both were significantly decreased with the co-addition of a PI3K inhibitor, confirming the key role of PI3K signaling in the downstream effects of CD5L binding (FIGS. 3D and 3E).

Next, as CD36 has been previously reported to be one of the major receptors for CD5L in macrophages, whether the effect of CD5L exogenous treatment on CD36 expression was sought to be determined. CD5L-treated RF24 cells had a significantly higher expression of CD36 than untreated RF24 cells (FIG. 3F), possibly through a positive feedback mechanism. To determine whether CD36 was implicated in the CD5L dependent upregulation of PI3K/AKT, RF24 cells were transfected with CD36 siRNA and again exogenously treated them with CD5L. Interestingly, the upregulation of p-AKT previously seen with exogenous CD5L treatment was negated under the conditions of CD36 receptor blockade (FIG. 3G). Also, exogenous CD5L treatment resulted in the same upregulation of PPAR-γ as previously demonstrated using the endogenous CD5L overexpressing construct (FIG. 3H). It is highly likely that the upregulation of the PI3K/AKT pathway is an integral component in the development of adaptive resistance to bevacizumab as RF24 cells exogenously treated with CD5L lost their sensitivity to bevacizumab compared to control cells (FIG. 3I). Moreover, treatment of RF24 cells with CD5L siRNA resulted in enhanced sensitivity to bevacizumab compared to siControl treated cells (FIG. 3J).

### Example 4—Silencing of PPAR-γ Inhibits Angiogenesis and Tumor Growth

To further explore the function of PPAR-γ and CD5L in tumor endothelial cells, murine ID8 ovarian cancer cells were injected intraperitoneally into C57BL/6 mice containing an endothelial cell-specific PPAR-γ knockout as well as C57BL/6 WT mice (FIG. 4A) (Guignabert et al., 2009). A 50% reduction in tumor weight and number of tumor nodules in the PPAR-γ KO mice compared with WT mice was observed (FIGS. 4B, 4C, and 4D). Moreover, immunohistochemical (IHC) analysis of tumor tissues from PPAR-γ KO versus WT mice revealed a significant decrease in cell proliferation and microvessel density in the PPAR-γ KO mice (FIGS. 4E and 4F). Next, a survival analysis of PPAR-γ KO mice vs WT mice during concurrent anti-VEGF treatment was performed. Mice harboring a PPAR-γ KO had significantly improved survival while receiving anti-VEGF treatment compared to the WT mice. Consistent with the in vitro data, tumor samples from PPAR-γ KO mice had lower p-AKT expression compared to tumors from WT mice (FIG. 4G).

### Example 5—Novel Antibodies Targeting CD5L Exhibit Anti-Tumor and Anti-Angiogenic Effect

Considering the finding that anti-VEGF therapy resistance is mediated in part by overexpression of CD5L, the inventors aimed to develop an antibody to specifically target CD5L and negate its downstream effects. To this end, a large panel (>350 binding hits) of anti-CD5L monoclonal antibodies was generated using two strategies by screening single B cells isolated from CD5L antigen immunized rabbit and by panning human antibody phage display libraries. Human CD5L protein was used for antibody generation and was expressed in HEK293 cells. The protein has a 6×HIS-tag and is purified to >95% purity using Ni-NTA resin (Sino Biologicals). Rabbits (NZW, Charles River) were immunized with the recombinantly produced CD5L using standard immunization procedures with 3 boost injections after primary priming immunization. Titer of anti-CD5L sera was determined by series of dilutions of serum in ELISA for binding on CD5L protein coated on 96-well plates (max-sorb plates, Nunc). When serum titer reached >10⁶, peripheral blood samples were collected from the immunized rabbits for B cell isolation from the freshly prepared peripheral blood mononuclear cells (PBMCs) using a fluorescence assisted cell sorting (FACS) instrument (BD FACSAria™ III, BD Biosciences). The sorted single B cells were collected in 96-well cell culture plates (Fisher Scientific) and were cultured for 7-10 days in a cell culture incubator with 5% CO₂ and 95% humidity in RPMI culture media with 10% FBS and added cytokins. The antibodies in the culture supernatants were assayed for CD5L bindings. Cells from the positives wells were lysed, total RNA was isolated, and cDNA was synthesized using a superscript reverse transcriptase II (Invitrogen) according to manufacturer's suggestion. DNA sequences of antibody variable regions from both heavy chains and light chains were amplified by polymerase chain reaction (PCR) using a set of designed primers and cloned into a vector for sequencing variable regions of each antibodies. Variable sequences of both DNA and amino acid sequences are listed in the Tables 3 and 4. CDRs of the anti-CD5L monoclonal antibodies were identified using the IMGT program (available on the world wide web at IMGT.org) and are listed in Table 2.

Selected CD5L binding hits were expressed as full-length human IgG or rabbit/human chimeric IgGs using a mammalian expression vector system in human embryonic kidney (HEK293) cells (Invitrogen). Antibodies were purified using protein A affinity resin by a fast protein liquid chromatography (FPLC). Purified CD5L binding antibodies were characterized for their biological properties.

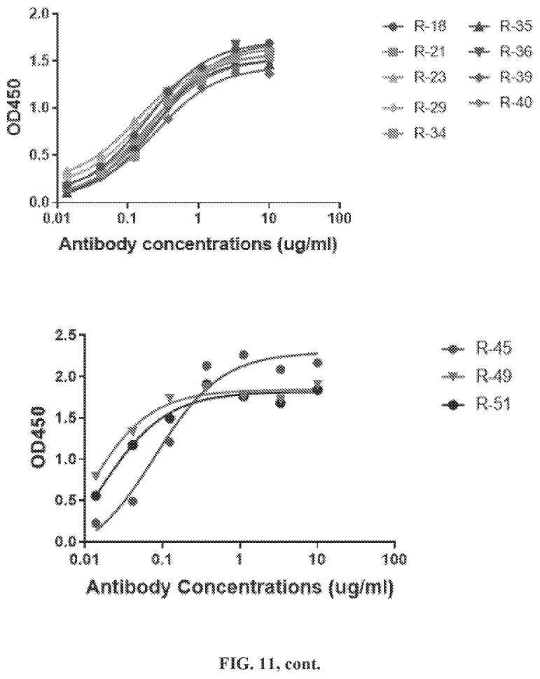

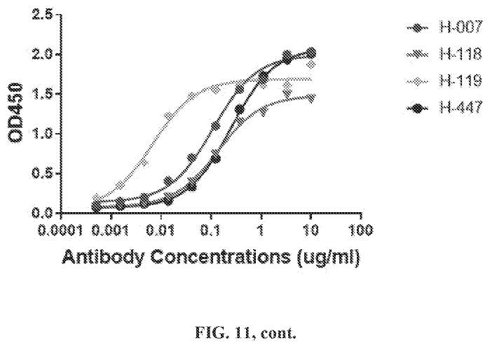

Binding of CD5L by monoclonal antibodies was first screened by ELISA using supernatants collected from the B cell cultures. ELISA concentration titration assay was used to determine the binding affinity of a panel of monoclonal antibodies to CD5L antigen (FIG. 11). Binding constants (EC₅₀) of a panel of monoclonal antibodies were estimated by titration ELISA, and the data were analyzed using the 4-parameter curve fitting with GraphPad Prism program (Table 6).

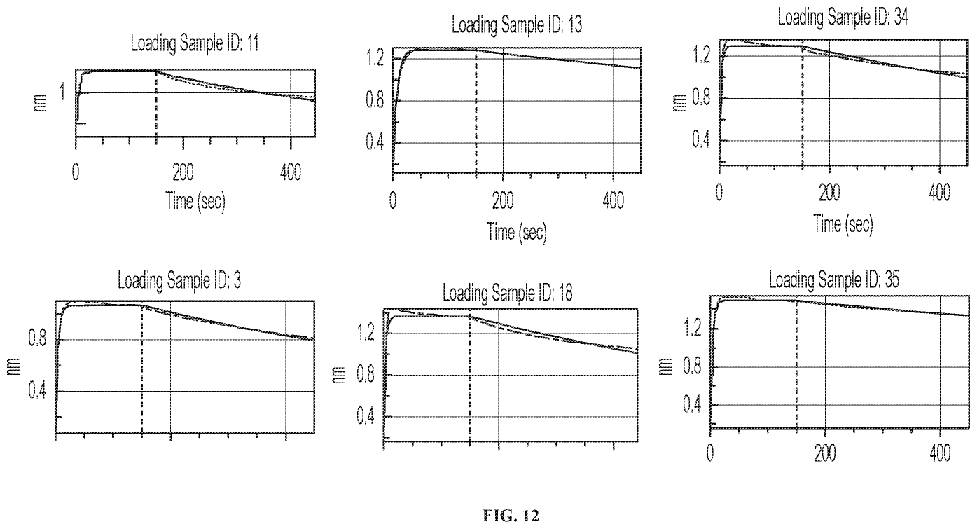

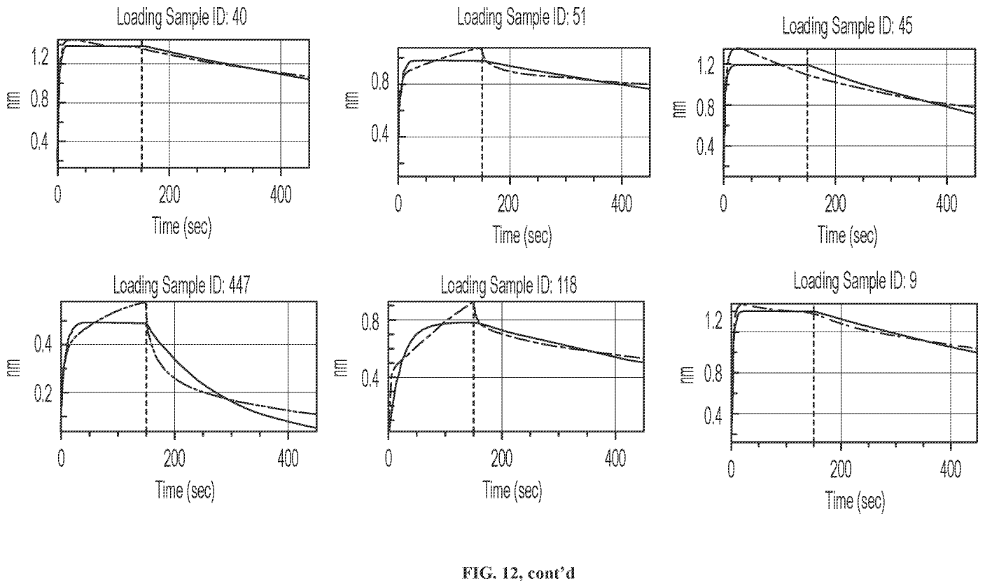

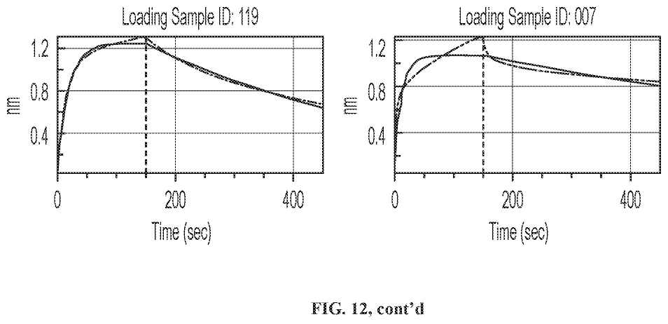

For antibody affinity measurement, antibody (30 μg/mL) was loaded onto the protein A biosensors for 4 min. Following a short baseline in kinetics buffer, the loaded biosensors were exposed to a series of recombinant CD5L protein at 0.1-200 nM and background subtraction was used to correct for sensor drifting. All experiments were performed with shaking at 1,000 rpm. Background wavelength shifts were measured from reference biosensors that were loaded only with antibody. Kinetic sensorgrams for each antibodies are shown in FIG. 12. ForteBio's data analysis software was used to fit the data to a 1:1 binding model to extract an association rate and dissociation rate. The K_{D} was calculated using the ratio of k_{off}/kₒₙ and the estimated values of K_{D} for CD5L mAbs in Table 7.

Pairwise binding competition among anti-CD5L mAbs was used to determine the binding epitopes of each mAbs using Octet instrument and protein A biosensors. The epitope bins are summarized in Table 8.

Based on in vitro characterization of binding affinity (KD), CD5L mouse cross reactivity, and binding epitopes, 10 antibodies were selected for further evaluation of antitumor efficacy. These 10 antibodies were screened in vivo using an orthotopic ovarian cancer mouse xenograft tumor model (SKOV3 ip1). Two monoclonal antibodies (H-447 and R-35) among those tested in vivo showed significant tumor reduction when compared with the isotype antibody control (FIGS. 5A, 5B, 5C, 5G, and 5H). Mice treated with isotype control antibody had a larger tumor burden than mice treated with an effective anti-CD5L antibody (FIG. 5A). Treatment with the two effective anti-CD5L antibodies resulted in significantly lower tumor weight and number of tumor nodules than control antibody (FIGS. 5B, 5C, 5G, and 5H). The anti-CD5L mAbs showed no effect on mouse body weight (FIG. 5I). Next, tumor blood vessel density from the in vivo specimens was interrogated, and it was found that treatment with the anti-CD5L antibody R-35 resulted in significantly fewer tumor blood vessels than treatment with control antibody (FIG. 5D). Similarly, the addition of R-35 antibody to RF24 cells treated with CD5L protein negated the observed increase in tube formation and cell migration when RF24 cells were treated with control antibody plus CD5L protein alone (FIGS. 5E and 5F).

TABLE 2
CDRs of light chain variable
sequences of anti-CD5L antibodies

| Antibody | Light Chains(LC) | | |
|---|---|---|---|
| Name | CDR1 | CDR2 | CDR3 |
| H-007 | QSISSY | AAS | QQSYSTPLT |
| | (SEQ ID NO: 1) | (SEQ ID | (SEQ ID NO: 3) |
| | | NO: 2) | |
| H-118 | QSISRY | AAS | QQSYRTPYT |
| | (SEQ ID NO: 4) | (SEQ ID | (SEQ ID NO: 5) |
| | | NO: 2) | |
| H-447 | QSISSY | AAS | QQSYSTLGT |
| | (SEQ ID NO: 1) | (SEQ ID | (SEQ ID NO: 6) |
| | | NO: 2) | |
| H-119 | QSINNY | AAS | QQSYSIPLT |
| | (SEQ ID NO: 7) | (SEQ ID | (SEQ ID NO: 8) |
| | | NO: 2) | |
| R-1 | QSVNKNNY | SAS(SEQ | QGTYYDSGWYNA |
| | (SEQ ID NO: 9) | ID NO: | (SEQ ID NO: 11) |
| | | 10) | |
| R-3 | ESIGNA | KAS | QRYYFNGVSVEIV |
| | (SEQ ID NO: 12) | (SEQ ID | (SEQ ID NO: 14) |
| | | NO: 13) | |
| R-4 | EDIYSY | EAS(SEQ | QNNFYMIGDDYGGT |
| | (SEQ ID NO: 15) | ID NO: | (SEQ ID NO: 17) |
| | | 16) | |
| R-5 | QSIGSN | TAS(SEQ | QCTYYGTSYMELGA |
| | (SEQ ID NO: 18) | ID NO: | (SEQ ID NO: 20) |
| | | 19) | |
| R-6 | ESINDR | ETS(SEQ | QSHDYISSSGMP |
| | (SEQ ID NO: 21) | ID NO: | (SEQ ID NO: 23) |
| | | 22) | |
| R-7 | QSLYNKKN | KAS | QGEFSCVAADCFS |
| | (SEQ ID NO: 24) | (SEQ ID | (SEQ ID NO: 25) |
| | | NO: 13) | |
| R-8 | ESIYSY | YTS(SEQ | QNTYGSSSNAFP |
| | (SEQ ID NO: 26) | ID NO: | (SEQ ID NO: 28) |
| | | 27) | |
| R-9 | EDIYSG | RAS(SEQ | QNNYGSSSNNYVFA |
| | (SEQ ID NO: 29) | ID NO: | ID NO: 31) |
| | | 30) | |
| R-10 | QSISSSY | KAS | LGGYFSFSNYDYG |
| | (SEQ ID NO: 32) | (SEQ ID | (SEQ ID NO: 33) |
| | | NO: 13) | |
| R-11 | QSIASR | RAS(SEQ | QSYYGSGYGKT |
| | (SEQ ID NO: 34) | ID NO: | (SEQ ID NO: 35) |
| | | 30) | |
| R-13 | ESISTY | RAS(SEQ | QQGYTYKNVDNA |
| | (SEQ ID NO: 36) | ID NO: | (SEQ ID NO: 37) |
| | | 30) | |
| R15 | HNINSA | KAS | QRHYFDGASADIA |
| | (SEQ ID NO: 38) | (SEQ ID | (SEQ ID NO: 39) |
| | | NO: 13) | |
| R-17 | QSIRSY | RAS(SEQ | QNVYYLSSSTYGA |
| | (SEQ ID NO: 40) | ID NO: | (SEQ ID NO: 41) |
| | | 30) | |
| R-18 | ESISVR | KAS | RNYYDISNNGAG |
| | (SEQ ID NO: 42) | (SEQ ID | (SEQ ID NO: 43) |
| | | NO: 13) | |

| | | | |
|---|---|---|---|
| R-20 | QSIGSS | RAS(SEQ | QNYISSSTEGND |
| | (SEQ ID NO: 44) | ID NO: | (SEQ ID NO: 45) |
| | | 30) | |
| R-21 | ESVYKSNY | AAA | QGTYYSSGWYNA |
| | (SEQ ID NO: 46) | (SEQ ID | (SEQ ID NO: 48) |
| | | NO: 47) | |
| R-22 | ESISSW | EAF(SEQ | QSAYYGGTYVGG |
| | (SEQ ID NO: 49) | ID NO: | (SEQ ID NO: 51) |
| | | 50) | |
| R-23 | HNINSA | KAS | QRHYFDGASADIA |
| | (SEQ ID NO: 38) | (SEQ ID | (SEQ ID NO: 39) |
| | | NO: 13) | |
| R-25 | QNNNNY | AAS | QCTDYGPTYLGT |
| | (SEQ ID NO: 52) | (SEQ ID | (SEQ ID NO: 53) |
| | | NO: 2) | |
| R-26 | QSIGSN | DAS | QQGDTYINVDNI |
| | (SEQ ID NO: 18) | (SEQ ID | (SEQ ID NO: 55) |
| | | NO: 54) | |
| R-28 | QSIDNY | RAS(SEQ | QSYYGSSTNA |
| | (SEQ ID NO: 56) | ID NO: | (SEQ ID NO: 57) |
| | | 30) | |
| R-29 | QSIYSS | GAS | QCSGYNSRV |
| | (SEQ ID NO: 58) | (SEQ ID | (SEQ ID NO: 60) |
| | | NO: 59) | |
| R-30 | QNIGSY | GAS | QCSGYNSRV: |
| | (SEQ ID NO: 61) | (SEQ ID | (SEQ ID NO: 60) |
| | | NO: 59) | |
| R-31 | ESIGTA | AAA | QFTYYGNSYGNA |
| | (SEQ ID NO: 62) | (SEQ ID | (SEQ ID NO: 63) |
| | | NO: 47) | |
| R-32 | ESINDR | ETS(SEQ | QSHDYISSSGMP |
| | (SEQ ID NO: 21) | ID NO: | (SEQ ID NO: 23) |
| | | 22) | |
| R-34 | QNIYNNR | QAS | AGGYSGTIFT |
| | (SEQ ID NO: 64) | (SEQ ID | (SEQ ID NO: 66) |
| | | NO: 65) | |
| R-35 | ERIYNN | SAS(SEQ | QCTGYGTTYGLV |
| | (SEQ ID NO: 67) | ID NO: | (SEQ ID NO: 68) |
| | | 10) | |
| R-36 | QTISSY | RAS(SEQ | QCPYYGSSHVGA |
| | (SEQ ID NO: 69) | ID NO: | (SEQ ID NO: 70) |
| | | 30) | |
| R-37 | QSISSY | YAS | QQGARSSNVDNI |
| | (SEQ ID NO: 1) | (SEQ ID | (SEQ ID NO: 72) |
| | | NO: 71) | |
| R-39 | QSIGSN | RAS(SEQ | QSYYGSSTNA |
| | (SEQ ID NO: 18) | ID NO: | (SEQ ID NO: 57) |
| | | 30) | |
| R-40 | ESVYKNNY | SAS(SEQ | AGYYGSSSVDRYT |
| | (SEQ ID NO: 73) | ID NO: | (SEQ ID NO: 74) |
| | | 10) | |
| R-41 | QTINNA | SAS(SEQ | AGYRPYINDGSA |
| | (SEQ ID NO: 75) | ID NO: | (SEQ ID NO: 76) |
| | | 10) | |
| R-42 | QSIGSN | TAS(SEQ | QCTYYGTSYMELGA |
| | (SEQ ID NO: 18) | ID NO: | (SEQ ID NO: 20) |
| | | 19) | |
| R-45 | QSIRTY | SAS(SEQ | QQGYYWSDVENT |
| | (SEQ ID NO: 77) | ID NO: | (SEQ ID NO: 78) |
| | | 10) | |
| R-49 | QSVDSNNY | KAS | AGGLGDNIYA |
| | N(SEQ ID O: 79) | (SEQ ID | (SEQ ID NO: 80) |
| | | NO: 13) | |
| R-51 | QSIYSS | ETS(SEQ | QSHDYISSSGMP |
| | (SEQ ID NO: 58) | ID NO: | (SEQ ID NO: 23) |
| | | 22) | |

TABLE 3
CDRs of heavy chain variable sequences of anti-CD5L antibodies

| Antibody | Heavy Chains(HC) | | |
|---|---|---|---|
| Name | CDR1 | CDR2 | CDR3 |
| H-007 | GYTFTGYY | INPNSGGT | AGGRIDAFDI |
| | (SEQ ID NO: 81) | (SEQ ID NO: 82) | (SEQ ID NO: 83) |
| H-118 | GYTFTGYY | INPNSGGT | ARRYSSSLRAFDI |
| | (SEQ ID NO: 81) | (SEQ ID NO: 82) | (SEQ ID NO: 84) |
| H-447 | GFTVSSNY | IYSGGST | ARESAVAGWGMDV |
| | (SEQ ID NO: 85) | (SEQ ID NO: 86) | (SEQ ID NO: 87) |
| H-119 | GYSFTSYW | IYPGDSDT | ARGLAASTRFDP |
| | (SEQ ID NO: 88) | (SEQ ID NO: 89) | (SEQ ID NO: 90) |
| R-1 | GIDLSNYA | ISSGDVT | ARFFAGRSESL |
| | (SEQ ID NO: 91) | (SEQ ID NO: 92) | (SEQ ID NO: 93) |
| R-3 | GIDLTSGT | IRGSGST | ARAFPGYNTFFNF |
| | (SEQ ID NO: 94) | (SEQ ID NO: 95) | (SEQ ID NO: 96) |
| R-4 | GFSLSSYY | ISDRGST | YRGVDGSGGYAAGDL |
| | (SEQ ID NO: 97) | (SEQ ID NO: 98) | (SEQ ID NO: 99) |
| R-5 | GFSLSSYA | IDVTGRT | GRDSYLDYGDPNGLDP |
| | (SEQ ID NO: 100) | (SEQ ID NO: 101) | (SEQ ID NO: 102) |
| R-6 | TIDVSKYA | ISSSGST | ARGGNGVAFKL |
| | (SEQ ID NO: 103) | (SEQ ID NO: 104) | (SEQ ID NO: 105) |
| R-7 | GFSLGRDA | INDDGDT | ARVGAEYLGWGNGFNL |
| | (SEQ ID NO: 106) | (SEQ ID NO: 107) | (SEQ ID NO: 108) |
| R-8 | GFSFSSSQY | IYGGSSGLT | ARAIGPTYYAFDP |
| | (SEQ ID NO: 109) | (SEQ ID NO: 110) | (SEQ ID NO: 111) |
| R-9 | GFYLSTHG | IVTSGNT | ARPGYKSVPL |
| | (SEQ ID NO: 112) | (SEQ ID NO: 113) | (SEQ ID NO: 114) |
| R-10 | GIDLSSYA | IGASGTT | ARAGYAGTGYPTRLDL |
| | (SEQ ID NO: 115) | (SEQ ID NO: 116) | (SEQ ID NO: 117) |
| R-11 | GFDFSSNT | ISYSGAT | ARDRGYVDGSYYGDFNL |
| | (SEQ ID NO: 118) | (SEQ ID NO: 119) | (SEQ ID NO: 120) |
| R-13 | GFSLSSYP | ITSGGSI | ARGYSSGTMMSSNL |
| | (SEQ ID NO: 121) | (SEQ ID NO: 122) | (SEQ ID NO: 123) |
| R-15 | GIDLSSNT | MRGGGST | ARAFPGYNSFFNF |
| | (SEQ ID NO: 124) | (SEQ ID NO: 125) | (SEQ ID NO: 126) |
| R-17 | GFSFSGGYN | IYAGSSGST | ARGAYAGGSYPPYFNL |
| | (SEQ ID NO: 127) | (SEQ ID NO: 128) | (SEQ ID NO: 129) |
| R-18 | GFSLSNYA | IVTSGST(SEQ ID | ARHAYSIGSSL |
| | (SEQ ID NO: 130) | (SEQ ID NO: 131) | (SEQ ID NO: 132) |
| R-20 | GIDLSSYA | ISSSGST | ARGSGGAVTYYGV |
| | (SEQ ID NO: 115) | (SEQ ID NO: 104) | (SEQ ID NO: 133) |
| R-21 | GIDLSSYA | ISSSDNT | ARFYAGRSYSL |
| | (SEQ ID NO: 115) | (SEQ ID NO: 134) | (SEQ ID NO: 135) |
| R-22 | GFSLSTYY | IRGGGEPV | ARRLDGFVRAYLDL |
| | (SEQ ID NO: 136) | (SEQ ID NO: 137) | (SEQ ID NO: 138) |
| R-23 | GIDLSSNT | MRGGGST | ARAFPGYNSFFNF |
| | (SEQ ID NO: 124) | (SEQ ID NO: 125) | (SEQ ID NO: 126) |
| R-25 | GFSLSNYA | SGYPGRT | ARHYGSTNGLNL |
| | (SEQ ID NO: 130) | (SEQ ID NO: 139) | (SEQ ID NO: 140) |
| R-26 | GFSFSSYYD | IYIGNSGNT | ARGASGYSGYGWFIL |
| | (SEQ ID NO: 141) | (SEQ ID NO: 142) | (SEQ ID NO: 143) |
| R-28 | GIDLSNYI | IASNGGT | ARGAGTTWSSFNL: |
| | (SEQ ID NO: 144) | (SEQ ID NO: 145) | (SEQ ID NO: 146) |
| R-29 | GFSLSSYA | IYASGTT | ARDSYSTGNL |
| | (SEQ ID NO: 100) | (SEQ ID NO: 147) | (SEQ ID NO: 148) |
| R-30 | GFSFSSGYW | IGARDSGSA(SEQ | ARSGASFHFKL |
| | (SEQ ID NO: 149) | ID NO: 150) | (SEQ ID NO: 151) |
| R-31 | GFSLSTYN | INGGGSA(SEQ ID | ARGYYAGADWNMPLDL |
| | (SEQ ID NO: 152) | NO: 153) | (SEQ ID NO: 154) |
| R-32 | GFSFSGGYD | IYAGSSGST(SEQ | ARGAYAGGGYPPYFNF |
| | (SEQ ID NO: 155) | ID NO: 128) | (SEQ ID NO: 156) |
| R-34 | GFSLSSSG | TFPSGSA(SEQ ID | AHVGATTGRDF |
| | (SEQ ID NO: 157) | NO: 158) | (SEQ ID NO: 159) |
| R-35 | GIDLSDYY | IYASGNA(SEQ ID | ARNIGGSSNL |
| | (SEQ ID NO: 160) | NO: 161) | (SEQ ID NO: 162) |
| R-36 | GFSLSKNYV | IYAGDGN(SEQ ID | ARWDYIGNNNL |
| | (SEQ ID NO: 163) | NO: 164) | (SEQ ID NO: 165) |
| R-37 | GIDLISYP | IATGGST(SEQ ID | ARRWSSAYDF |
| | (SEQ ID NO: 166) | NO: 167) | (SEQ ID NO: 168) |
| R-39 | GIDLSSYT | IAINGGT(SEQ ID | ARGAGKTWSSFNL |
| | (SEQ ID NO: 169) | NO: 170) | (SEQ ID NO: 171) |
| R-40 | GIDLSRYS | MSIGSSA(SEQ ID | ARSNLYRGAFDP |
| | (SEQ ID NO: 172) | NO: 173) | (SEQ ID NO: 174) |
| R-41 | GFSFSSSQD | IGFGSGNT(SEQ ID | ARDPFTVAGDGDNL |
| | (SEQ ID NO: 175) | NO: 176) | (SEQ ID NO: 177) |
| R-42 | GFSLSSYA | IDVTGRT(SEQ ID | GRDSYLDYGDPNGLDP |
| | (SEQ ID NO: 100) | NO: 101) | (SEQ ID NO: 102) |
| R-45 | GFSINDFH | IGTGGSA(SEQ ID | ARRDWSDGGYSLL |
| | (SEQ ID NO: 178) | NO: 179) | (SEQ ID NO: 180) |
| R-49 | GFSLSINA | ITNIGNT(SEQ ID | ARNSYFDP |
| | (SEQ ID NO: 181) | NO: 182) | (SEQ ID NO: 183) |
| R-51 | TIDVSKYA | ISSSGST(SEQ ID | ARGGNGVAFKL |
| | (SEQ ID NO: 103) | NO: 104) | (SEQ ID NO: 105) |

TABLE 4
Protein sequences for anti-CD5L antibody variable regions

| Antibody | Chain | Variable Sequence (5′ to 3′) | SEQ ID NO: |
|---|---|---|---|
| H-007 | Heavy | EVQLVQSGAEVKKPGASVKVSCKASGYTFTGYYMHWVRQ | 184 |
| | | APGQGLEWMGWINPNSGGTNYAQKFQGRVTMTRDTSIST | |
| | | AYMELSRLRSDDTAVYYCAGGRIDAFDIWGQGTTVSVSS | |
| | Light | DIQMTQSPSSLSASVGDRVTITCRASQSISSYLNWYQQK | 185 |
| | | PGKAPKLLIYAASSLQSGVPSRFSGSGSGTDFTLTISSL | |
| | | QPEDFATYYCQQSYSTPLTFGGGTKVDIK | |
| H-118 | Heavy | EVQLVQSGAEVKKPGASVKVSCKASGYTFTGYYMHWVRQ | 186 |
| | | APGQGLEWMGRINPNSGGTNYAQKFQGRVTMTRDTSIST | |
| | | AYMELSRLXSDDTAVYYCARRYSSSLRAFDIWGQGTMVS | |
| | | VSS | |
| | Light | DIQMTQSPSSLSASVGDRVSITCRASQSISRYLNWYQQK | 187 |
| | | PGKAPKLLIYAASSLQSGVPSRFSGSGSGTDFTLTISSL | |
| | | QPEDFATYYCQQSYRTPYTFSQGTKLEIK | |
| H-447 | Heavy | EVQLVQSGGGLVQPGGSLRLSCAASGFTVSSNYMSWVRQ | 188 |
| | | APGKGLEWVSVIYSGGSTYYADSVKGRFTISRHNSKNTL | |
| | | YLQMNSLRAEDTAVYYCARESAVAGWGMDVWGKGTTVTV | |
| | | SS | |
| | Light | DIQMTQSPSSLSASVGDRVTITCRASQSISSYLNWYQQK | 189 |
| | | PGKAPKLLIYAASSLQSGVPSRFSGSGSGTDFTLTISSL | |
| | | QPEDFATYYCQQSYSTLGTFGQGTKLEIK | |
| H-119 | Heavy | EVQLVQSGAEVKKPGESLKISCKGSGYSFTSYWIGWVRQ | 190 |
| | | MPGKGLEWMGIIYPGDSDTRYSPSFQGQVTISADKSIST | |
| | | AYLQWSSLKASDTAMYYCARGLAASTRFDPWGQGTLVTV | |
| | | SS | |
| | Light | DVQMTQSPSSLSASVGDRVTITCRPSQSINNYLNWYQQK | 191 |
| | | PGEAPKLLIYAASNLQSGVPSRFSGSGSGTDFTLTISSL | |
| | | QPEDFGTYYCQQSYSIPLTFGGGTTVEIK | |
| R-1 | Heavy | QSVEESGGRLVTPGTPLTLTCTVSGIDLSNYAINWVRQA | 192 |
| | | PGKGLEWIGMISSGDVTYYASWAKGRFIISRTSSTTVDL | |
| | | KMTSPTIEDTATYFCARFFAGRSESLWGQGTLVTVSS | |
| | Light | ELDLTQTPSSVSAAVGGTVTINCQSSQSVNKNNYLSWYQ | 193 |
| | | QKPGQPPKLLIYSASALASGVSSRFSGSGSGTEFTLTIS | |
| | | DVQCDDAATYYCQGTYYDSGWYNAFGGGTEVEIK | |
| R-3 | Heavy | QSLKESGGRLVKPDETLTITCTASGIDLTSGTVGWFRQA | 194 |
| | | PGKGLEWIGIIRGSGSTYYATWAKGRFTISKTSTTVDLK | |
| | | LTSPTTEDTATYLCARAFPGYNTFFNFWGQGTLVTISS | |
| | Light | ELDLTQTPSSVEAAVGGTVTIKCQASESIGNALAWYQQK | 195 |
| | | PGQPPKLLIWKASNLESGVPPRFKGSGSGTEFTLTISDL | |
| | | ECADAATYYCQRYYFNGVSVEIVFGGGTEVVVKG | |
| R-4 | Heavy | QSVEESGGRLVTPGTPLTLTCTASGFSLSSYYMTWVRQA | 196 |
| | | PGKGLEYIGIISDRGSTYYASWAKGRFTIAKTSSTTVDL | |
| | | KLTSLTTEDTATYFCYRGVDGSGGYAAGDLWGQGTLVTI | |
| | | SS | |
| | Light | ELVLTQTPASVEAAVGGTVTIKCQASEDIYSYFAWYQQK | 197 |
| | | PGQRPRLLIYEASKVASGVPSRFKGGGSGTQFTLTISDL | |
| | | ECADAATYYCQNNFYMIGDDYGGTFGGGTEVEIK | |
| R-5 | Heavy | QSVKESGGRLVTPGTPLTLTCTVSGFSLSSYAMSWVRQA | 198 |
| | | PGKGLEWIGVIDVTGRTYYASWAKGRFTISKTSTTVDLK | |
| | | ITSPTTEDTATYFCGRDSYLDYGDPNGLDPWGPGTLVTI | |
| | | SS | |
| | Light | ELVMTQTPASVEAAVGGTVTIKCQASQSIGSNLAWYQQK | 199 |
| | | PGQPPKLLIYTASNLAPGVSSRFKGSRSGTESTLIISDL | |
| | | ECADAATYYCQCTYYGTSYMELGAFGGGTELEIK | |
| R-6 | Heavy | QSLEESGGRLVTPGTPLTLTCTVSTIDVSKYAVIWVRQA | 200 |
| | | PGKGLEYIGIISSSGSTYYASWAKGRFTISKTSSTTVDL | |
| | | KMTSPTAEDTATYFCARGGNGVAFKLWGQGTLVTISS | |
| | Light | ELDMTQTPASVEAAVGGTVTINCQASESINDRLAWYQLK | 201 |
| | | PGQPPKLLIYETSQVESGVPSRFSGSGSGTQFTLTISDL | |
| | | ECADAATYYCQSHDYISSSGMPFGGGTEVVVK | |
| R-7 | Heavy | QSVKESGGRLVTPGTPLTLTCTMSGFSLGRDALTWVRQA | 202 |
| | | PGKGLQWIAFINDDGDTDYPNWARGRFTISKTSSTTVDL | |
| | | KITSPTTEDTATYFCARVGAEYLGWGNGFNLWGQGTLVT | |
| | | ISS | |
| | Light | ELDMTQTPASVSAAVGGTVTINCQASQSLYNKKNLAWYQ | 203 |
| | | QKPGQPPKLLIYKASTLASGVPSRFEGSGSGTEYALTIS | |
| | | GLQCDDAATYYCQGEFSCVAADCFSFGGGTEVVVK | |
| R-8 | Heavy | QSVKESGGDLVKPGASLTLTCTASGFSFSSSQYMCWVRQ | 204 |
| | | APGKGLEWIACIYGGSSGLTNYATWAKGRFTVSKTSSTT | |
| | | VTLQMTSLTDADTATYFCARAIGPTYYAFDPWGPGTLVT | |
| | | VSS | |
| | Light | ELDLTQTPSSVSAAVGDTVTIKCQASESIYSYLAWYQQK | 205 |
| | | PGQPPKLLIYYTSTLASGVPSRFKGSGSGTEFTLTISDL | |
| | | ECADAATYYCQNTYGSSSNAFPFGGGTEVEIK | |
| R-9 | Heavy | QSVKESGGRLVTPGTPLTLTCTVSGFYLSTHGMTWVRQA | 206 |
| | | PGKGLEYIGVIVTSGNTYYATWATGRFTISKTSTTVDLK | |
| | | ITSPTTEDTATYFCARPGYKSVPLWGQGTLVTISS | |
| | Light | ELDLTQTPASVSAAVGGTVTINCQASEDIYSGLTWYQQK | 207 |
| | | PGQPPKLLIYRASTLASGVPPRFKGSGSGTEFTLTISDL | |
| | | ECDDAATYYCQNNYGSSSNNYVFAFGGGTEVEIK | |
| R-10 | Heavy | QSVKESGGRLVTPGTPLTLTCTLSGIDLSSYAMGWVRQA | 208 |
| | | PGKGLEYIGIIGASGTTYYANWAKGRFTISRTSTTVDLK | |
| | | ITSPTTEDTATYFCARAGYAGTGYPTRLDLWGQGTLVTI | |
| | | SS | |
| | Light | ELDMTQTPASVEAAVGGTVTIKCQASQSISSSYLAWYQQ | 209 |
| | | KPGQPPKLLIYKASTLESGVPSRFSGSGSGTEFTLTISG | |
| | | VQCDDAATYYCLGGYFSFSNYDYGFGGGTEVEIK | |
| R-11 | Heavy | QSVKESGGDLVKPGASLTLTCTASGFDFSSNTMCWVRQA | 210 |
| | | PGKGLEYIGYISYSGATHYASWAKGRFTISKTLSTTVTL | |
| | | QMTSLTDADTATYFCARDRGYVDGSYYGDFNLWGPGTLV | |
| | | TVSS | |
| | Light | ELVMTQTPSSASEPVGGTVTIKCQASQSIASRLAWFQQK | 211 |
| | | PGQPPKLLIYRASTLESGVSSRFKGSGFGTEFTLTISDL | |
| | | ECADAATYYCQSYYGSGYGKTFGGGTDVVVK | |
| R-13 | Heavy | QSLEESGGRLVTPGTPLTLTCTVSGFSLSSYPMSWVRQA | 212 |
| | | PGKGLEWIGIITSGGSIFYATWANGRFTISKTSTTVDLK | |
| | | ITSPTTEDTATYFCARGYSSGTMMSSNLWGQGTLVTVSS | |
| | Light | ELVLTQTPASVSEPVGGTVTIKCQASESISTYLSWYQQK | 213 |
| | | PGQRPKLLIYRASTLESGVPSRFKGSGSGTEYTLTISGV | |
| | | QCADAATYYCQQGYTYKNVDNAFGGGTEVVVK | |
| R-15 | Heavy | QSLEESGGRLVMPGGSLTLTCTVSGIDLSSNTMGWFRQA | 214 |
| | | PGKGLEWIGIMRGGGSTYYATWAKGRFTISKTSTTVDLK | |
| | | LTSPTTEDTATYFCARAFPGYNSFFNFWGQGTLVTISS | |
| | Light | TQTPASVSEPVGGTVTIKCQASHNINSALAWYQQKPGQP | 215 |
| | | PKLLIYKASTLASGVPPRFKGSGSGTEFTLTISDLECAD | |
| | | AATYYCQRHYFDGASADIAFGGGTEVEIK | |
| R-17 | Heavy | QSVXESGGGLVQPEGSLTLTCTASGFSFSGGYNMCWVRQ | 216 |
| | | APGKGLEWIACIYAGSSGSTYYASWAKGRFTISKTSSTT | |
| | | VTLQMTSLTAADTATYFCARGAYAGGSYPPYFNLWGPGT | |
| | | LVTISS | |
| | Light | EXXXTQTPASVSEPVGGTVTIKCQASQSIRSYLAWYQQK | 217 |
| | | AGQPPKLLIYRASTLASGVPSRFKGSGSGTEYTLTISDL | |
| | | ECADAATYYCQNVYYLSSSTYGAFGGGTEVVVK | |
| R-18 | Heavy | QSVKESGGRLVTPGTPLTLTCTVSGFSLSNYAMSWVRQA | 218 |
| | | PGKGLEWIGIIVTSGSTYYASWAKGRFTISKTSTTVDLK | |
| | | MTSLTAADTATYFCARHAYSIGSSLWGQGTLVTISS | |
| | Light | ELDLTQTPPSASEPVGGTVTIKCQASESISVRLAWYQQK | 219 |
| | | PGQPPKLLIYKASNLPSGVPSRFKGSGSGTEFTLTISDL | |
| | | ECDDAATYYCRNYYDISNNGAGFGGGTEVEIK | |
| R-20 | Heavy | QSVKESGGGLFKPTDTLTLTCTVSGIDLSSYAMTWVRQA | 220 |
| | | PGEGLEWIGTISSSGSTYYASWAKGRFTISKTSSTTVDL | |
| | | KMTSLTAADTATYFCARGSGGAVTYYGVWGQGTLVTVSS | |
| | Light | ELDLTQTPASVSEPVGGTVTIKCQASQSIGSSLAWYQQK | 221 |
| | | PGQPPKLLIYRASTLASGVPSRFSGSGSGTEFTLTISDL | |
| | | ECAHAATYYCQNYISSSTFGNDFGGGTEVVVK | |
| R-21 | Heavy | QSVEESGGRLVTPGTPLTLTCTVSGIDLSSYAINWVRQA | 222 |
| | | PGEGLEWIGMISSSDNTYYANWAKGRFTISRDSTTVDLK | |
| | | ITSPTTEDTATYFCARFYAGRSYSLWGQGTLVTISS | |
| | Light | ELDLTQTPASVSAAVGGTVTISCQSSESVYKSNYLSWYQ | 223 |
| | | QKAGQPPKLLIYAAATLASGVPSRFKGSGSGTQFTLTIS | |
| | | GVQCDDAATYYCQGTYYSSGWYNAFGGGTEVVVK | |
| R-22 | Heavy | EQSVEESGGRLVTPGTPLTLTCTASGFSLSTYYMSWVRQ | 224 |
| | | APGKGLEWIGIIRGGGEPVWYADWAKGRFTISRISTTVD | |
| | | LKITSPTPEDTATYFCARRLDGFVRAYLDLWGQGTLVTV | |
| | | SS | |
| | Light | TQTPASVEAAVGGTVTIKCQASESISSWLAWYQQKPGQP | 225 |
| | | PKLLIYEAFSLASGVPSRFSGSGSGTEFTLTISDLECDD | |
| | | AAIYYCQSAYYGGTYVGGFGGGTEVVVK | |
| R-23 | Heavy | QSVEESGGRLVMPGGSLTLTCTVSGIDLSSNTMGWFRQA | 226 |
| | | PGKGLEWIGIMRGGGSTYYATWAKGRFTISKTSTTVDLK | |
| | | LTSPTTEDTATYFCARAFPGYNSFFNFWGQGTLVTISS | |
| | Light | ELDLTQTPASVSEPVGGTVTIKCQASHNINSALAWYQQK | 227 |
| | | PGQPPKLLIYKASTLASGVPPRFKGSGSGTEFTLTISDL | |
| | | ECADAATYYCQRHYFDGASADIAFGGGTEVVVK | |
| R-25 | Heavy | AAVKESGGRLVTPGTPLTLTCTVSGFSLSNYAMGWVRQP | 228 |
| | | PGKGLEWIGFSGYPGRTYYASWAKGRFTISKTSTTVDLK | |
| | | ITSPTAEDTATYFCARHYGSTNGLNLWGQGTLVTVSS | |
| | Light | ELDLTQTPASVSEPVGGTVTIKCQASQNNNNYLAWYQQK | 229 |
| | | PGQPPKLLIYAASSLASGVPSRFKGSGSGTQFTLTISDL | |
| | | ECDDAATYYCQCTDYGPTYLGTFGGGTEVEIK | |
| R-26 | Heavy | QSLEESGGDLVKPGASLTLTCTASGFSFSSYYDMCWVRQ | 230 |
| | | APGKGLEWIACIYIGNSGNTYYASWAKGRFTISRTSSTT | |
| | | VDLKMSSLTAADTASYFCARGASGYSGYGWFILWGPGTL | |
| | | VTISS | |
| | Light | ELVMTQTPASVSAAVGGTVTIKCQASQSIGSNLAWYQQK | 231 |
| | | PGQRPKLLIYDASDLASGVPSRFKGSGSGPEYTLTISGV | |
| | | ECDDAATYYCQQGDTYINVDNIFGGGTEVEIK | |
| R-28 | Heavy | QSVEESGGRLVTPGTPLTLTCTVSGIDLSNYIMGWVRQA | 232 |
| | | PGKGLEWIGIIASNGGTVYANWAKGRFTISKTSTTVDLK | |
| | | MTSPTPEDTATYFCARGAGTTWSSFNLWGQGTLVTISS | |
| | Light | ELVLTQTPASVSEPVGGTVTINCQVSQSIDNYLNWYHQK | 233 |
| | | PGQPPKRLIYRASTLASGVPSRFRGSGSGTQFTLTISDL | |
| | | ECADAATYYCQSYYGSSTNAFGGGTEVVVK | |
| R-29 | Heavy | QSVEESGGRLVTPGTPLTLTCTVSGFSLSSYAMNWVRQA | 234 |
| | | PGKGLEWIGLIYASGTTHYASWVNGRFTISKTSTTVTLK | |
| | | MTSLTAADTATYFCARDSYSTGNLWGQGTLVTISS | |
| | Light | ELVLTQTPASVSEPVGGTVTIKCQASQSIYSSLVWYQQK | 235 |
| | | PGQPPKLLIYGASNLESGVPSRFKGSGSGTEYTLTISGV | |
| | | QCADAATYYCQCSGYNSRVFGGGTEVEIK | |
| R-30 | Heavy | SSVEESGGGLVKPGASLTLTCTASGFSFSSGYWMCWVRQ | 236 |
| | | APGKELEWIACIGARDSGSAYYATWAKGRFTISKISSTT | |
| | | VTLQMTSLTAADTATYFCARSGASFHFKLWGPGTLVTIS | |
| | | S | |
| | Light | ELVLTQTPASVSEPVGGTVTIKCQASQNIGSYLAWYQQK | 237 |
| | | PGQPPKLLIYGASNLESGVPSRFKGSGSGTEYTLTISGV | |
| | | QCADAATYYCQCSGYNSRVFGGGTEVVVK | |
| R-31 | Heavy | QSLEESGGDLVTPGTPLTLTCTVSGFSLSTYNIQWVRQA | 238 |
| | | PGKGLECIGFINGGGSAYYASWVNGRFTVSKASTTVTLK | |
| | | ITAPTTEDTATYFCARGYYAGADWNMPLDLWGQGTLVTV | |
| | | SS | |
| | Light | ELVLTQTPSPVSAGVGGTVTIKCQSSESIGTALAWYQQK | 239 |
| | | LGQPPKLLIYAAALLASGVSSRFKGSGSGTEYTLTISDL | |
| | | ECDDAATYYCQFTYYGNSYGNAFGGGTEVEIK | |
| R-32 | Heavy | QSVKESGGDLVKPEGSLTLTCTASGFSFSGGYDMCWVRQ | 240 |
| | | APGKGLEWIACIYAGSSGSTYYASWAKGRFTISKTSSTT | |
| | | VTLQMTSLTAADTATYFCARGAYAGGGYPPYFNFWGPGT | |
| | | LVTISS | |
| | Light | ELVMTQTPASVEAAVGGTVTINCQASESINDRLAWYQLK | 241 |
| | | PGQPPKLLIYETSKVESGVPSRFSGSGSGTQFTLTISDL | |
| | | ECADAATYYCQSHDYISSSGMPFGGGTELEIK | |
| R-34 | Heavy | QSVKESGGRLVTPGTPLTLTCTVSGFSLSSSGMSWVRQA | 242 |
| | | PGKGLEWIGLTFPSGSASYASWAKGRFTVSRISTTVDLN | |
| | | LTSPTTEDTATYFCAHVGATTGRDFWGQGTLVTVSS | |
| | Light | ELVMTQTASSVSAAVGGTVTISCQSSQNIYNNRWSWYQQ | 243 |
| | | KPGQPPKLLIYQASTLASGVPSRFKGSGSGTQFTLTISG | |
| | | VQCDDAATYYCAGGYSGTIFTFGGGTELEIK | |
| R-35 | Heavy | AAVKESGGRLVTPGTPLTLTCTVSGIDLSDYYMTWVRQA | 244 |
| | | PGKGLEYIGIIYASGNAYFASWAKGRFTISRTSSTTVDL | |
| | | NVASPTTEDTATYFCARNIGGSSNLWGQGTLVTVSS | |
| | Light | ELDMTQTPASVSEPVGGTVTIKCQASERIYNNLAWYQQK | 245 |
| | | PGQPPKLLFYSASTLASGVPSRFKGSGSGTEFTLTISDL | |
| | | ECADAAIYYCQCTGYGTTYGLVFGGGTEVEI | |
| R-36 | Heavy | QSVKESGGGLFQPGGSLALTCKASGFSLSKNYVMCWVRQ | 246 |
| | | APGKGLEWIACIYAGDGNTYYVSWAKGRFTISKTSSTTV | |
| | | TLQMTSLTAADTATYFCARWDYIGNNNLWGPGTLVTISS | |
| | Light | ELVLTQTPASVEAAVGGTVTIKCQASQTISSYLAWYQQK | 247 |
| | | PGQPPKLLIYRASTLASGVPSRFKGSGSGTQFTLTISDL | |
| | | ECADGATYYCQCPYYGSSHVGAFGGGTEVVVK | |
| R-37 | Heavy | QSVKESGGRLVTPGTPLTLTCTVSGIDLISYPMGWVRQA | 248 |
| | | PGKGLEYIGIIATGGSTYYASWAKGRFTISTSSTTVDLK | |
| | | MTSLTAADTATYFCARRWSSAYDFWGQGTLVTISS | |
| | Light | ELDLTQTPASVEVAVGGTVTIKCQASQSISSYLAWYQQK | 249 |
| | | PGQPPKLLIYYASTLASGVPSRFSGSGSGTEFTLTISGV | |
| | | QCDDAATYYCQQGARSSNVDNIFGGGTEVVVK | |
| R-39 | Heavy | QSLEESGGRLVTPGGSLTLTCTVSGIDLSSYTMGWVRQA | 250 |
| | | PGKGLEWIGVIAINGGTVYASWAKGRFTISKTSTTVDLK | |
| | | MTSPTPEDTATYFCARGAGKTWSSFNLWGQGTLVTISS | |
| | Light | ELVLTQTPASVSEPVGGTVTIKCQASQSIGSNLAWYQQK | 251 |
| | | PGQPPKLLIYRASTLESGVPSRFSGSGSGTEFTLTISDL | |
| | | ECADAATYYCQSYYGSSTNAFGGGTEVVVK | |
| R-40 | Heavy | QSVKESGGRLVKPDETLTITCTVSGIDLSRYSIQWVRQA | 252 |
| | | PGGGLQWIGVMSIGSSAYYANWAKGRFTISKTSSTTVDL | |
| | | TMTSLTAADTATYFCARSNLYRGAFDPWGPGTLVTISS | |
| | Light | ELVMTQTPASVEAAVGGTVTIKCQSSESVYKNNYLSWYQ | 253 |
| | | LKPGQPPKLLFYSASTLASRVPSRFSGSGSGTQFTLTIS | |
| | | DLECDDAATYYCAGYYGSSSVDRYTFGGGTEVEIK | |
| R-41 | Heavy | QLMESGGGLVQPGASLTLTCKASGFSFSSSQDMCWVRQA | 254 |
| | | PGKGPEWIGCIGFGSGNTWYASWVNGRFTISRSTSLNTV | |
| | | ELKMTGLTAADTATYFCARDPFTVAGDGDNLWGPGTLVT | |
| | | VSS | |
| | Light | ELDLTQTPASVSEPVGGTVTIKCQASQTINNALAWYQQK | 255 |
| | | PGQPPKLLIYSASTLASGVSSRFSGSGSGTQFTLTISGV | |
| | | ECDDAATYYCAGYRPYINDGSAFGGGTEVVVK | |
| R-42 | Heavy | QSVEESGGRLVTPGTPLTLTCTVSGFSLSSYAMSWVRQA | 256 |
| | | PGKGLEWIGVIDVTGRTYYASWAKGRFTISKTSTTVDLK | |
| | | ITSPTTEDTATYFCGRDSYLDYGDPNGLDPWGPGTLVTI | |
| | | SS | |
| | Light | ELVMTQTPASVEAAVGGTVTIKCQASQSIGSNLAWYQQK | 257 |
| | | PGQPPKLLIYTASNLAPGVSSRFKGSRSGTESTLIISDL | |
| | | ECADAATYYCQCTYYGTSYMELGAFGGGTEVVVK | |
| R-45 | Heavy | QSVKESGGRLVTPGTPLTLTCTVSGFSINDFHMCWVRQA | 258 |
| | | PGKGLEYIGMIGTGGSAYYATWTKGRFTISRTSTTVDLK | |
| | | ITSPTTEDTATYFCARRDWSDGGYSLLWGQGTLVTVSS | |
| | Light | ELDLTQTPASVSEPVGGTVTIKCQASQSIRTYLNWYQQK | 259 |
| | | PGQPPKLLIYSASTLASGIPSRFKGSGSGTQFTLTISDL | |
| | | ECADAATYYCQQGYYWSDVENTFGGGTEVEIN | |
| R-49 | Heavy | QSVKESGGRLVTPGTPLTLTCTVSGFSLSINAINWVRQA | 260 |
| | | PGKGLEWIGIITNIGNTYYASWAKGRFTISRTSTTVDLT | |
| | | MTSLTTEDTATYFCARNSYFDPWGPGTLVTISS | |
| | Light | ELVLTQTPSPVSAAVGGTVSISCQASQSVDSNNYCSWYQ | 261 |
| | | QKPGQPPKLLIHKASKLASGVPSRFKGSGSGTQFTLTIN | |
| | | DLECDDAATYYCAGGLGDNIYAFGGGTEVEIN | |
| R-51 | Heavy | QSVKESGGRLVTPGTPLTLTCTVSTIDVSKYAVIWVRQA | 262 |
| | | PGKGLEYIGIISSSGSTYYASWAKGRFTISKTSSTTVDL | |
| | | KMTSPTAEDTATYFCARGGNGVAFKLWGQGTLVTISS | |
| | Light | ELDLTQTPASVEAAVGGTVTINCQASQSIYSSLAWYQQK | 263 |
| | | PGQPPKLLIYETSKVESGVPSRFSGSGSGTQFTLTISDL | |
| | | ECADAATYYCQSHDYISSSGMPFGGGTELEIL | |

TABLE 5
Nucleotide sequence for anti-CD5L antibody variable regions

| Antibody | Chain | Variable Sequence (5′ to 3′) | SEQ ID NO: |
|---|---|---|---|
| H-007 | Heavy | ACAGGTGTCCACTCGGAGGTGCAGCTGGTGCAGTCTGGG | 264 |
| | | GCTGAGGTGAAGAAGCCTGGGGCCTCAGTGAAGGTCTCC | |
| | | TGCAAGGCTTCTGGATACACCTTCACCGGCTACTATATG | |
| | | CACTGGGTGCGACAGGCCCCTGGACAAGGGCTTGAGTGG | |
| | | ATGGGATGGATCAACCCTAACAGTGGTGGCACAAACTAT | |
| | | GCACAGAAGTTTCAGGGCAGGGTCACCATGACCAGGGAC | |
| | | ACGTCCATCAGCACAGCCTACATGGAGCTGAGCAGGCTG | |
| | | AGATCTGACGACACGGCCGTGTATTACTGTGCGGGCGGA | |
| | | AGGATTGATGCTTTTGATATCTGGGGCCAAGGGACCACG | |
| | | GTCAGCGTCTCCTCAGCATCCACCAAGGGC | |
| | Light | ACAGACGCTCGCTGCGACATCCAGATGACCCAGTCTCCA | 265 |
| | | TCCTCCCTGTCTGCATCTGTAGGAGACAGAGTCACCATC | |
| | | ACTTGCCGGGCAAGTCAGAGCATTAGCAGCTATTTAAAT | |
| | | TGGTATCAGCAGAAACCAGGGAAAGCCCCTAAGCTCCTG | |
| | | ATCTATGCTGCATCCAGTTTGCAAAGTGGGGTCCCATCA | |
| | | AGGTTCAGTGGCAGTGGATCTGGGACAGATTTCACTCTC | |
| | | ACCATCAGCAGTCTGCAACCTGAAGATTTTGCAACTTAC | |
| | | TACTGTCAACAGAGTTACAGTACCCCGCTCACTTTCGGC | |
| | | GGAGGGACTAAAGTGGATATCAAACGTACGGTGGCTGCA | |
| H-118 | Heavy | ACAGGTGTCCACTCGGAAGTGCAGCTGGTGCAGTCTGGG | 266 |
| | | GCTGAGGTGAAGAAGCCTGGGGCCTCAGTGAAGGTCTCC | |
| | | TGCAAGGCTTCTGGATACACCTTCACCGGCTACTATATG | |
| | | CACTGGGTGCGACAGGCCCCTGGACAAGGGCTTGAGTGG | |
| | | ATGGGACGGATCAACCCTAACAGTGGTGGCACAAACTAT | |
| | | GCACAGAAGTTTCAGGGCAGGGTCACCATGACCAGGGAC | |
| | | ACGTCCATCAGCACAGCCTACATGGAGCTGAGCAGGCTG | |
| | | ANATCTGACGACACGGCCGTGTATTACTGTGCGAGAAGG | |
| | | TATAGCAGCAGCTTACGCGCTTTTGATATCTGGGGCCAA | |
| | | GGGACAATGGTCAGCGTCTCCTCAGCATCCACCAAGGGC | |
| | Light | ACAGACGCTCGCTGCGACATCCAGATGACCCAGTCTCCA | 267 |
| | | TCCTCCCTGTCTGCATCTGTAGGAGACAGAGTCTCCATC | |
| | | ACTTGCCGGGCAAGTCAGAGCATTAGTAGATATTTAAAT | |
| | | TGGTATCAGCAGAAACCAGGGAAAGCCCCTAAGCTCCTG | |
| | | ATCTATGCTGCATCCAGTTTGCAAAGTGGGGTCCCATCA | |
| | | AGGTTCAGTGGCAGTGGATCTGGGACAGATTTCACTCTC | |
| | | ACCATCAGTAGTCTGCAACCTGAAGATTTTGCAACTTAT | |
| | | TACTGTCAACAGAGTTACAGGACCCCCTACACTTTTAGC | |
| | | CAGGGGACCAAGCTGGAGATCAAACGTACGGTGGCTGCA | |
| H-447 | Heavy | ACAGGTGTCCACTCGGAAGTGCAGCTGGTGCAGTCTGGA | 268 |
| | | GGAGGCTTGGTCCAGCCTGGGGGGTCCCTGAGACTCTCC | |
| | | TGTGCAGCCTCTGGGTTCACCGTCAGTAGCAACTACATG | |
| | | AGCTGGGTCCGCCAGGCTCCAGGGAAGGGGCTGGAGTGG | |
| | | GTCTCAGTTATTTATAGCGGTGGTAGCACATACTACGCA | |
| | | GACTCCGTGAAGGGCCGATTCACCATCTCCAGACACAAT | |
| | | TCCAAGAACACGCTGTATCTTCAAATGAACAGCCTGAGA | |
| | | GCTGAGGACACGGCCGTGTATTACTGTGCGAGAGAATCG | |
| | | GCAGTGGCCGGGTGGGGTATGGACGTCTGGGGCAAAGGG | |
| | | ACCACGGTCACCGTCTCTTCAGCATCCACCAAGGGC | |
| | Light | ACAGACGCTCGCTGCGACATCCAGATGACCCAGTCTCCA | 269 |
| | | TCCTCCCTGTCTGCATCTGTAGGAGACAGAGTCACCATC | |
| | | ACTTGCCGGGCAAGTCAGAGCATTAGCAGCTATTTAAAT | |
| | | TGGTATCAGCAGAAACCAGGGAAAGCCCCTAAGCTCCTG | |
| | | ATCTATGCTGCATCCAGTTTGCAAAGTGGGGTCCCATCA | |
| | | AGGTTCAGTGGCAGTGGATCTGGGACAGATTTCACTCTC | |
| | | ACCATCAGCAGTCTGCAACCTGAAGATTTTGCAACTTAC | |
| | | TACTGTCAACAGAGTTACAGTACCCTCGGGACGTTCGGC | |
| | | CAAGGGACCAAGCTGGAAATCAAACGTACGGTGGCTGCA | |
| H-119 | Heavy | ACAGGTGTCCACTCGGAGGTGCAGCTGGTGCAGTCTGGA | 270 |
| | | GCAGAGGTGAAAAAGCCCGGGGAGTCTCTGAAGATCTCC | |
| | | TGTAAGGGTTCTGGATACAGCTTTACCAGCTACTGGATC | |
| | | GGCTGGGTGCGCCAGATGCCCGGGAAAGGCCTGGAGTGG | |
| | | ATGGGGATCATCTATCCTGGTGACTCTGATACCAGATAC | |
| | | AGCCCGTCCTTCCAAGGCCAGGTCACCATCTCAGCCGAC | |
| | | AAGTCCATCAGCACCGCCTACCTGCAGTGGAGCAGCCTG | |
| | | AAGGCCTCGGACACCGCCATGTATTACTGTGCGAGAGGT | |
| | | CTCGCAGCATCGACTCGGTTCGACCCCTGGGGCCAGGGC | |
| | | ACCCTGGTCACCGTCTCCTCAGCATCCACCAAGGGC | |
| | Light | ACAGACGCTCGCTGCGACGTCCAGATGACCCAGTCTCCA | 271 |
| | | TCCTCCCTGTCTGCATCTGTCGGAGACAGAGTCACCATC | |
| | | ACTTGCCGGCCAAGTCAGAGCATTAACAACTATTTAAAT | |
| | | TGGTATCAGCAGAAACCAGGGGAAGCCCCTAAGCTCCTG | |
| | | ATCTATGCTGCATCCAATTTGCAAAGTGGGGTCCCATCA | |
| | | AGGTTCAGTGGCAGTGGATCCGGGACAGATTTCACTCTC | |
| | | ACCATCAGCAGTCTGCAACCTGAAGATTTTGGAACTTAC | |
| | | TACTGTCAACAGAGTTACAGTATCCCTCTCACTTTCGGC | |
| | | GGAGGGACTACAGTGGAAATCAAACGTACGGTGGCTGCA | |
| R-1 | Heavy | ACAGGTGTCCACTCGGAGCAGTCGGTGGAGGAGTCCGGG | 272 |
| | | GGTCGCCTGGTCACGCCGGGGACACCCCTGACACTCACC | |
| | | TGCACAGTCTCTGGAATCGACCTCAGTAATTATGCAATA | |
| | | AACTGGGTCCGCCAGGCTCCAGGGAAGGGGCTGGAATGG | |
| | | ATCGGAATGATTAGTAGTGGTGATGTCACATACTACGCG | |
| | | AGCTGGGCGAAAGGCCGATTCATCATCTCCAGAACCTCG | |
| | | TCGACCACGGTGGATCTGAAAATGACCAGTCCGACAATC | |
| | | GAGGACACGGCCACCTATTTCTGTGCCAGATTTTTCGCT | |
| | | GGTAGGAGTGAATCCTTGTGGGGCCAAGGCACCCTGGTC | |
| | | ACCGTCTCTTCAGGGCAACCTAAGGCT | |
| | Light | ACAGATGCCAGATGCGAGCTCGATCTGACCCAGACACCA | 273 |
| | | TCCTCCGTGTCTGCCGCTGTGGGAGGCACAGTCACCATC | |
| | | AACTGCCAGTCCAGTCAGAGTGTTAATAAGAACAACTAC | |
| | | TTATCCTGGTATCAACAAAAACCAGGGCAGCCTCCCAAG | |
| | | CTCCTAATCTATTCTGCATCCGCTCTGGCATCTGGGGTC | |
| | | TCATCGCGGTTCAGCGGCAGTGGATCTGGGACAGAGTTC | |
| | | ACTCTCACCATCAGCGACGTGCAGTGTGACGATGCTGCC | |
| | | ACTTACTACTGTCAAGGCACTTATTATGATAGTGGTTGG | |
| | | TACAATGCTTTCGGCGGAGGGACCGAGGTGGAAATCAAA | |
| | | GGTGATCCAGTTGCAC | |
| R-3 | Heavy | ACAGGTGTCCACTCGCAGTCGTTGAAGGAGTCCGGGGGT | 274 |
| | | CGCCTGGTCAAGCCTGACGAAACCCTGACAATCACCTGC | |
| | | ACAGCCTCTGGAATCGACCTCACTAGCGGTACAGTGGGC | |
| | | TGGTTCCGCCAGGCTCCAGGGAAGGGACTAGAATGGATC | |
| | | GGAATTATTCGTGGTAGTGGTAGCACATATTACGCGACC | |
| | | TGGGCGAAAGGCCGATTCACCATCTCCAAAACCTCGACC | |
| | | ACGGTGGATCTGAAGCTCACCAGTCCGACAACCGAGGAC | |
| | | ACGGCCACCTATCTCTGTGCCAGAGCCTTCCCTGGTTAT | |
| | | AATACTTTCTTTAACTTCTGGGGCCAAGGCACCCTGGTC | |
| | | ACCATCTCTTCAGGGCAACCTAAGGCT | |
| | Light | ACAGATGCCAGATGCGAGCTCGATCTGACCCAGACACCA | 275 |
| | | TCCTCCGTGGAGGCAGCTGTGGGAGGCACAGTCACCATC | |
| | | AAGTGTCAGGCCAGTGAGAGCATTGGCAATGCATTAGCC | |
| | | TGGTATCAGCAGAAACCAGGGCAGCCTCCCAAACTCCTG | |
| | | ATCTGGAAGGCATCCAATCTAGAATCTGGGGTCCCCCCG | |
| | | CGGTTCAAAGGCAGTGGATCTGGGACAGAGTTCACTCTC | |
| | | ACCATTAGCGACCTGGAATGTGCCGATGCTGCCACTTAC | |
| | | TATTGTCAAAGGTATTATTTCAATGGTGTTAGTGTTGAG | |
| | | ATAGTTTTCGGCGGAGGGACCGAGGTGGTGGTCAAAGGT | |
| | | GATCCAGTTGCAC | |
| R-4 | Heavy | ACAGGTGTCCACTCGGAGCAGTCGGTGGAGGAGTCCGGG | 276 |
| | | GGTCGCCTGGTCACGCCTGGGACACCCCTGACACTCACC | |
| | | TGCACAGCCTCTGGATTCTCCCTCAGTAGCTACTACATG | |
| | | ACCTGGGTCCGCCAGGCTCCAGGGAAGGGGCTGGAATAC | |
| | | ATCGGAATCATTAGTGATCGTGGTAGCACATACTACGCG | |
| | | AGCTGGGCGAAAGGCCGATTCACCATCGCCAAAACCTCG | |
| | | TCGACCACGGTGGATCTGAAACTGACCAGTCTGACAACC | |
| | | GAGGACACGGCCACCTATTTCTGTTATAGAGGTGTTGAT | |
| | | GGTAGTGGTGGGTATGCTGCCGGTGACTTGTGGGGCCAA | |
| | | GGCACCCTGGTCACCATCTCTTCAGGGCAACCTAAGGCT | |
| | Light | ACAGATGCCAGATGCGAGCTCGTGCTGACCCAGACACCA | 277 |
| | | GCCTCCGTGGAGGCAGCTGTGGGAGGCACAGTCACCATC | |
| | | AAGTGTCAGGCCAGTGAGGACATTTATAGCTACTTCGCC | |
| | | TGGTATCAGCAGAAACCAGGGCAGCGTCCCAGGCTCCTG | |
| | | ATCTATGAAGCATCCAAAGTGGCATCTGGGGTCCCATCG | |
| | | CGGTTCAAAGGCGGTGGATCTGGGACACAATTCACTCTC | |
| | | ACCATCAGCGACCTGGAGTGTGCCGATGCTGCCACTTAC | |
| | | TACTGTCAAAACAATTTTTATATGATTGGTGATGATTAT | |
| | | GGTGGTACTTTCGGCGGAGGGACCGAGGTGGAAATCAAA | |
| | | GGTGATCCAGTTGCAC | |
| R-5 | Heavy | ACAGGTGTCCACTCGCAGTCGGTGAAGGAGTCCGGGGGT | 278 |
| | | CGCCTGGTCACGCCTGGGACACCCCTGACACTCACCTGC | |
| | | ACAGTCTCTGGATTCTCCCTCAGTAGTTATGCAATGAGC | |
| | | TGGGTCCGCCAGGCTCCAGGGAAGGGGCTGGAGTGGATC | |
| | | GGAGTCATTGATGTTACTGGTAGGACATACTACGCGAGC | |
| | | TGGGCGAAAGGCCGATTCACCATCTCCAAAACCTCGACC | |
| | | ACGGTGGATCTGAAAATCACCAGTCCGACAACCGAGGAC | |
| | | ACGGCCACCTATTTCTGTGGCAGAGATAGCTACCTTGAC | |
| | | TATGGTGACCCCAATGGTCTTGATCCCTGGGGCCCAGGC | |
| | | ACCCTGGTCACCATCTCCTCAGGGCAACCTAAGGCT | |
| | Light | ACAGATGCCAGATGCGAGCTCGTGATGACCCAGACACCA | 279 |
| | | GCCTCCGTGGAGGCAGCTGTGGGAGGCACAGTCACCATC | |
| | | AAGTGCCAGGCCAGTCAGAGCATTGGTAGTAATTTAGCC | |
| | | TGGTATCAGCAGAAACCAGGGCAGCCTCCCAAGCTCCTG | |
| | | ATCTATACTGCATCCAATCTGGCGCCTGGAGTCTCATCG | |
| | | CGGTTCAAAGGCAGTAGATCTGGGACAGAGTCCACTCTC | |
| | | ATCATCAGCGACCTGGAGTGTGCCGATGCTGCCACTTAC | |
| | | TACTGTCAATGTACTTATTATGGTACTAGTTATATGGAA | |
| | | TTAGGGGCTTTCGGCGGAGGGACCGAGCTGGAAATCAAA | |
| | | GGTGATCCAGTTGCAC | |
| R-6 | Heavy | ACAGGTGTCCACTCGCAGTCGTTGGAGGAGTCCGGGGGT | 280 |
| | | CGCCTGGTCACGCCTGGGACACCCCTGACACTCACCTGC | |
| | | ACAGTCTCTACAATCGACGTCAGTAAGTATGCAGTGATC | |
| | | TGGGTCCGCCAGGCTCCAGGGAAGGGGCTGGAATACATC | |
| | | GGAATCATTAGTAGTAGTGGTAGCACATACTACGCGAGC | |
| | | TGGGCGAAAGGCCGATTCACCATCTCCAAAACCTCATCG | |
| | | ACCACGGTGGATCTGAAAATGACCAGTCCGACAGCCGAG | |
| | | GACACGGCCACCTATTTCTGTGCCAGAGGGGGTAATGGT | |
| | | GTCGCCTTTAAATTATGGGGCCAAGGCACCCTGGTCACC | |
| | | ATCTCTTCAGGGCAACCTAAGGCT | |
| | Light | ACAGATGCCAGATGCGAGCTCGATATGACCCAGACACCA | 281 |
| | | GCCTCCGTGGAGGCAGCTGTGGGAGGCACAGTCACCATC | |
| | | AATTGCCAGGCCAGTGAGAGCATTAACGATAGATTAGCC | |
| | | TGGTATCAGCTGAAACCAGGGCAGCCTCCCAAACTCCTG | |
| | | ATCTACGAAACATCCCAAGTGGAATCTGGGGTCCCATCG | |
| | | CGGTTCAGCGGCAGTGGATCTGGGACACAGTTCACTCTC | |
| | | ACCATCAGCGACCTGGAGTGTGCCGATGCTGCCACTTAC | |
| | | TACTGTCAAAGCCATGATTATATTAGTAGTAGTGGTATG | |
| | | CCTTTCGGCGGAGGGACCGAGGTGGTGGTCAAAGGTGAT | |
| | | CCAGTTGCAC | |
| R-7 | Heavy | ACAGGTGTCCACTCGCAGTCGGTGAAGGAGTCCGGGGGT | 282 |
| | | CGCCTGGTCACGCCTGGGACACCCCTGACACTCACCTGC | |
| | | ACAATGTCTGGATTCTCCCTCGGTAGGGATGCGCTGACC | |
| | | TGGGTCCGCCAGGCTCCAGGAAAGGGGCTACAATGGATC | |
| | | GCATTCATCAATGATGATGGTGACACTGACTATCCAAAC | |
| | | TGGGCGAGAGGCCGATTCACCATCTCCAAAACCTCGTCG | |
| | | ACCACGGTGGATCTGAAAATCACCAGTCCTACAACCGAG | |
| | | GACACGGCCACCTATTTCTGTGCCAGAGTGGGGGCTGAA | |
| | | TATCTTGGTTGGGGTAATGGTTTTAATTTGTGGGGCCAA | |
| | | GGCACCCTGGTCACCATCTCCTCAGGGCAACCTAAGGCT | |
| | Light | ACAGATGCCAGATGCGAGCTCGATATGACCCAGACACCA | 283 |
| | | GCCTCCGTGTCTGCAGCTGTGGGAGGCACAGTCACCATC | |
| | | AACTGCCAGGCCAGTCAGAGTCTTTATAATAAGAAAAAT | |
| | | TTAGCCTGGTATCAGCAGAAACCAGGGCAGCCTCCCAAG | |
| | | CTCCTGATCTACAAGGCTTCCACTCTGGCATCTGGGGTC | |
| | | CCATCGCGGTTCGAAGGCAGTGGATCTGGGACAGAGTAC | |
| | | GCTCTCACCATCAGCGGCTTGCAGTGTGACGATGCTGCC | |
| | | ACTTACTACTGTCAAGGCGAATTTAGTTGTGTTGCTGCT | |
| | | GATTGTTTTTCTTTCGGCGGAGGGACCGAGGTGGTGGTC | |
| | | AAAGGTGATCCAGTTGCAC | |
| R-8 | Heavy | ACAGGTGTCCACTCGCAGTCGGTGAAGGAGTCCGGGGGA | 284 |
| | | GACCTGGTCAAGCCTGGGGCATCCCTGACACTCACCTGT | |
| | | ACAGCCTCTGGATTCTCCTTCAGTAGCAGCCAGTACATG | |
| | | TGCTGGGTCCGCCAGGCTCCAGGGAAGGGGCTGGAGTGG | |
| | | ATCGCATGCATTTATGGTGGTAGTAGTGGTCTCACAAAC | |
| | | TACGCGACCTGGGCGAAAGGCCGATTCACCGTCTCCAAA | |
| | | ACCTCGTCGACCACGGTGACTCTGCAAATGACCAGTCTG | |
| | | ACAGACGCAGACACGGCCACGTATTTCTGTGCGAGAGCT | |
| | | ATTGGTCCTACTTATTATGCTTTTGATCCCTGGGGCCCA | |
| | | GGCACCCTGGTCACCGTCTCTTCAGGGCAACCTAAGGCT | |
| | Light | ACAGATGCCAGATGCGAGCTCGATCTGACCCAGACTCCA | 285 |
| | | TCCTCCGTGTCTGCAGCTGTGGGAGACACAGTCACCATC | |
| | | AAGTGCCAGGCCAGTGAGAGCATTTACAGTTACTTAGCC | |
| | | TGGTATCAGCAGAAACCAGGGCAGCCTCCCAAGCTCCTG | |
| | | ATCTATTATACATCCACTCTGGCATCTGGGGTCCCATCG | |
| | | CGGTTCAAAGGCAGTGGATCTGGGACAGAGTTCACTCTC | |
| | | ACCATCAGCGACCTGGAGTGTGCCGATGCTGCCACTTAC | |
| | | TACTGTCAAAACACTTATGGTAGTAGTAGTAATGCTTTT | |
| | | CCTTTTGGCGGAGGGACCGAGGTGGAAATCAAAGGTGAT | |
| | | CCAGTTGCAC | |
| R-9 | Heavy | ACAGGTGTCCACTCGCAGTCGGTGAAGGAGTCCGGGGGT | 286 |
| | | CGCCTGGTCACGCCTGGGACACCCCTGACACTCACCTGC | |
| | | ACAGTCTCTGGATTCTACCTCAGTACCCATGGAATGACC | |
| | | TGGGTCCGCCAGGCTCCAGGGAAGGGGCTGGAATACATC | |
| | | GGAGTCATTGTTACTAGTGGTAACACATACTACGCGACC | |
| | | TGGGCGACTGGCCGATTCACCATCTCCAAAACCTCGACC | |
| | | ACGGTGGATCTGAAAATCACCAGTCCGACAACCGAGGAC | |
| | | ACGGCCACCTATTTCTGTGCCAGACCTGGTTATAAAAGT | |
| | | GTCCCCTTGTGGGGCCAAGGCACCCTGGTCACCATCTCT | |
| | | TCAGGGCAACCTAAGGCT | |
| | Light | ACAGATGCCAGATGCGAGCTCGATCTGACCCAGACACCA | 287 |
| | | GCCTCCGTGTCTGCAGCTGTGGGAGGCACAGTCACCATC | |
| | | AATTGCCAGGCCAGTGAGGACATTTACAGTGGTTTGACC | |
| | | TGGTATCAGCAGAAACCAGGGCAGCCTCCCAAGCTCCTG | |
| | | ATCTACAGGGCATCCACTCTGGCATCTGGGGTCCCACCG | |
| | | CGGTTCAAAGGCAGTGGATCTGGGACAGAATTCACTCTC | |
| | | ACCATCAGCGACCTGGAGTGTGACGATGCTGCCACTTAC | |
| | | TACTGTCAAAACAATTATGGTAGTAGTAGTAATAATTAT | |
| | | GTTTTTGCTTTCGGCGGAGGGACCGAGGTGGAAATCAAA | |
| | | GGTGATCCAGTTGCAC | |
| R-10 | Heavy | ACAGGTGTCCACTCGCAGTCGGTGAAGGAGTCCGGGGGT | 288 |
| | | CGCCTGGTCACGCCTGGGACACCCCTGACACTCACCTGT | |
| | | ACACTCTCTGGAATCGACCTCAGTAGCTATGCAATGGGC | |
| | | TGGGTCCGCCAGGCTCCAGGGAAGGGGCTGGAATACATC | |
| | | GGAATCATTGGTGCTAGTGGTACCACTTACTACGCGAAC | |
| | | TGGGCGAAAGGCCGATTCACCATCTCCAGAACCTCGACC | |
| | | ACGGTGGATCTGAAAATCACCAGTCCGACAACCGAGGAC | |
| | | ACGGCCACCTATTTCTGTGCCAGAGCTGGTTATGCTGGT | |
| | | ACTGGTTATCCTACTCGTTTGGATCTCTGGGGCCAGGGC | |
| | | ACCCTGGTCACCATCTCTTCAGGGCAACCTAAGGCT | |
| | Light | ACAGATGCCAGATGCGAGCTCGATATGACCCAGACACCA | 289 |
| | | GCCTCCGTGGAGGCAGCTGTGGGAGGCACAGTCACCATC | |
| | | AAGTGCCAGGCCAGTCAGAGTATTAGTAGTAGCTACTTA | |
| | | GCCTGGTATCAGCAGAAACCAGGGCAGCCTCCCAAGCTC | |
| | | CTGATCTACAAGGCATCCACTCTGGAATCTGGGGTCCCA | |
| | | TCGCGGTTCAGCGGCAGTGGATCTGGGACAGAGTTCACT | |
| | | CTCACCATCAGCGGCGTGCAGTGTGACGATGCTGCCACT | |
| | | TACTACTGTCTAGGCGGTTATTTTAGTTTTAGTAATTAT | |
| | | GATTATGGTTTCGGCGGAGGGACCGAGGTGGAAATCAAA | |
| | | GGTGATCCAGTTGCAC | |
| R-11 | Heavy | ACAGGTGTCCACTCGCAGTCGGTGAAGGAGTCCGGGGGA | 290 |
| | | GACCTGGTCAAGCCTGGGGCATCCCTGACACTCACCIGC | |
| | | ACAGCCTCTGGATTCGACTTCAGTAGCAATACAATGTGC | |
| | | TGGGTCCGCCAGGCTCCAGGGAAAGGGCTGGAATATATT | |
| | | GGATACATTAGTTATAGTGGTGCTACGCATTACGCGAGC | |
| | | TGGGCGAAAGGCCGATTCACCATCTCCAAAACCTTGTCG | |
| | | ACCACGGTGACTCTGCAAATGACCAGTCTGACAGACGCG | |
| | | GACACGGCCACCTATTTCTGTGCGAGAGATCGGGGTTAT | |
| | | GTTGATGGTAGTTATTATGGTGACTTTAACTTGTGGGGC | |
| | | CCAGGCACCCTGGTCACCGTCTCTTCAGGGCAACCTAAG | |
| | | GCT | |
| | Light | ACAGATGCCAGATGCGAGCTCGTGATGACCCAGACTCCA | 291 |
| | | TCTTCCGCGTCTGAACCTGTGGGAGGCACAGTCACCATC | |
| | | AAGTGCCAGGCCAGTCAGAGCATTGCTAGTAGATTAGCC | |
| | | TGGTTTCAGCAGAAACCAGGGCAGCCTCCCAAGCTCCTA | |
| | | ATATACAGGGCATCCACTCTGGAATCTGGGGTCTCATCG | |
| | | CGGTTCAAAGGCAGTGGATTTGGGACAGAGTTCACTCTC | |
| | | ACCATCAGCGACCTGGAGTGTGCCGATGCTGCCACTTAC | |
| | | TACTGTCAAAGCTATTATGGTAGTGGTTATGGCAAGACT | |
| | | TTCGGCGGAGGGACCGACGTGGTGGTCAAAGGTGATCCA | |
| | | GTTGCAC | |
| R-13 | Heavy | ACAGGTGTCCACTCGCAGTCGCTGGAGGAGTCCGGGGGT | 292 |
| | | CGCCTGGTCACGCCTGGGACACCCCTGACACTCACCTGC | |
| | | ACAGTCTCTGGATTCTCCCTCAGTAGCTATCCAATGAGC | |
| | | TGGGTCCGCCAGGCTCCAGGGAAGGGGCTGGAATGGATC | |
| | | GGAATCATTACTAGTGGTGGTAGTATATTTTACGCGACC | |
| | | TGGGCGAATGGTCGATTCACCATCTCCAAAACCTCGACC | |
| | | ACGGTGGATCTGAAAATCACCAGTCCGACAACCGAGGAC | |
| | | ACGGCCACCTATTTCTGTGCCAGAGGTTATAGTAGTGGT | |
| | | ACTATGATGTCTTCTAACTTGTGGGGCCAAGGCACCCTG | |
| | | GTCACCGTCTCCTCAGGGCAACCTAAGGCT | |
| | Light | ACAGATGCCAGATGCGAGCTCGTGCTGACCCAGACACCA | 293 |
| | | GCCTCCGTGTCTGAACCTGTGGGAGGCACAGTCACCATC | |
| | | AAGTGCCAGGCCAGTGAGAGCATTAGTACTTACTTATCC | |
| | | TGGTATCAGCAGAAACCAGGGCAGCGTCCCAAGCTCCTG | |
| | | ATCTACAGGGCATCCACTCTGGAATCTGGGGTCCCATCG | |
| | | CGGTTCAAAGGCAGTGGATCTGGGACAGAGTACACTCTC | |
| | | ACCATTAGCGGCGTGCAGTGTGCCGATGCTGCCACTTAC | |
| | | TACTGTCAACAGGGTTATACTTATAAAAATGTTGATAAT | |
| | | GCTTTCGGCGGAGGGACCGAGGTGGTGGTCAAAGGTGAT | |
| | | CCAGTTGCAC | |
| R-15 | Heavy | ACAGGTGTCCACTCGGAGCAGTCGTTGGAGGAGTCCGGG | 294 |
| | | GGTCGTCTGGTCATGCCTGGAGGATCCCTGACACTCACC | |
| | | TGCACAGTCTCTGGAATCGACCTCAGTAGCAATACAATG | |
| | | GGCTGGTTCCGCCAGGCTCCAGGGAAGGGGCTGGAATGG | |
| | | ATCGGGATTATGCGTGGTGGTGGTAGCACATACTACGCG | |
| | | ACCTGGGCGAAAGGCCGATTCACCATCTCCAAAACCTCG | |
| | | ACCACGGTGGATCTGAAACTCACCAGTCCGACAACCGAG | |
| | | GACACGGCCACCTATTTCTGTGCCAGAGCCTTCCCTGGT | |
| | | TATAATTCTTTCTTTAACTTCTGGGGCCAAGGCACCCTG | |
| | | GTCACCATCTCTTCAGGGCAACCTAAGGCT | |
| | Light | ACAGATGCCAGATGCACCCAGACACCAGCCTCCGTGTCT | 295 |
| | | GAACCTGTGGGAGGCACAGTCACCATCAAGTGCCAGGCC | |
| | | AGTCACAACATTAATAGTGCTTTGGCCTGGTATCAGCAG | |
| | | AAACCAGGGCAGCCTCCCAAGCTCCTGATCTACAAGGCA | |
| | | TCCACTCTGGCATCTGGGGTCCCACCGCGGTTCAAAGGC | |
| | | AGTGGATCTGGGACAGAGTTCACTCTCACCATCAGCGAC | |
| | | CTGGAGTGTGCCGATGCTGCCACTTACTACTGTCAAAGG | |
| | | CATTATTTCGATGGTGCCAGTGCTGATATAGCTTTCGGC | |
| | | GGAGGGACCGAGGTGGAAATCAAAGGTGATCCAGTTGCA | |
| | | C | |
| R-17 | Heavy | ACAGGTGTCCACTCGCAGTCGGTGNAGGAGTCCGGGGGA | 296 |
| | | GGCCTGGTCCAGCCTGAGGGATCCCTGACACTCACCTGC | |
| | | ACAGCCTCTGGATTCTCCTTCAGTGGCGGCTACAACATG | |
| | | TGCTGGGTCCGCCAGGCTCCAGGGAAGGGGCTGGAGTGG | |
| | | ATCGCATGCATTTATGCTGGTAGTAGTGGTAGCACTTAC | |
| | | TACGCGAGCTGGGCGAAAGGCCGATTCACCATCTCCAAA | |
| | | ACCTCGTCAACCACGGTGACTCTGCAAATGACCAGTCTG | |
| | | ACAGCCGCGGACACGGCCACCTATTTCTGTGCGAGAGGG | |
| | | GCTTATGCTGGTGGTAGTTACCCCCCCTACTTTAACTTG | |
| | | TGGGGCCCAGGCACCCTGGTCACCATCTCCTCAGGGCAA | |
| | | CCTAAGGTT | |
| | Light | ACAGATGCCAGATGCGAGCNCCTNNNGACCCAGACACCA | 297 |
| | | GCCTCCGTGTCTGAGCCTGTGGGAGGCACAGTCACCATC | |
| | | AAGTGCCAGGCCAGTCAGAGCATTAGAAGTTACTTAGCC | |
| | | TGGTATCAGCAGAAAGCAGGGCAGCCTCCCAAGCTCCTG | |
| | | ATCTACAGGGCATCCACTCTGGCATCIGGGGTCCCATCG | |
| | | CGGTTCAAAGGCAGTGGATCTGGGACAGAGTACACTCTC | |
| | | ACCATCAGCGACCTGGAGTGTGCCGATGCTGCCACTTAC | |
| | | TACTGCCAAAATGTTTATTATCTTAGTAGTAGTACATAT | |
| | | GGTGCTTTCGGCGGAGGGACCGAGGTGGTGGTCAAAGGT | |
| | | GATCCAGTTGCAC | |
| R-18 | Heavy | ACAGGTGTCCACTCGCGAGCAGTCGGTGAAGGAGTCCGG | 298 |
| | | GGGTCGCCTGGTCACGCCTGGGACACCCCTGACACTCAC | |
| | | CTGCACAGTCTCTGGATTCTCCCTCAGTAACTATGCAAT | |
| | | GAGCTGGGTCCGCCAGGCTCCAGGGAAGGGGCTGGAATG | |
| | | GATCGGAATCATTGTTACTAGTGGTAGCACATACTACGC | |
| | | GAGCTGGGCAAAAGGCCGATTCACCATCTCCAAAACCTC | |
| | | GACCACGGTGGATCTGAAAATGACCAGTCTGACAGCCGC | |
| | | GGACACGGCCACCTATTTCTGTGCCAGACATGCGTATAG | |
| | | TATTGGTTCGAGCTTGTGGGGCCAAGGCACCCTGGTCAC | |
| | | CATCTCTTCAGGGCAACCTAAGGCT | |
| | Light | ACAGATGCCAGATGCGAGCTCGATCTGACCCAGACACCA | 299 |
| | | CCTTCTGCGTCTGAACCTGTGGGAGGCACAGTCACCATC | |
| | | AAGTGCCAGGCCAGTGAGAGCATCAGTGTTAGATTAGCC | |
| | | TGGTATCAGCAGAAACCAGGGCAGCCTCCCAAGCTCCTG | |
| | | ATCTACAAGGCATCCAATCTGCCATCTGGGGTCCCATCG | |
| | | CGGTTCAAAGGCAGTGGATCTGGGACAGAGTTCACTCTC | |
| | | ACCATCAGCGACCTGGAGTGTGACGATGCTGCCACTTAT | |
| | | TACTGTCGCAACTATTATGATATTAGTAATAATGGTGCT | |
| | | GGTTTTGGCGGAGGGACCGAGGTGGAAATCAAAGGTGAT | |
| | | CCAGTTGCAC | |
| R-20 | Heavy | ACAGGTGTCCACTCGGAGCAGTCGGTGAAGGAGTCCGGG | 300 |
| | | GGAGGCCTCTTCAAGCCAACGGATACCCTGACACTCACC | |
| | | TGCACAGTCTCTGGAATCGACCTCAGTAGCTATGCAATG | |
| | | ACCTGGGTCCGCCAGGCTCCAGGGGAGGGGCTGGAGTGG | |
| | | ATCGGAACCATTAGTAGTAGTGGTAGCACATACTACGCG | |
| | | AGCTGGGCGAAAGGCCGATTCACCATCTCCAAAACCTCG | |
| | | TCGACCACGGTGGATCTGAAAATGACCAGTCTGACAGCC | |
| | | GCGGACACGGCCACCTATTTCTGTGCCAGAGGATCTGGT | |
| | | GGTGCTGTTACTTATTATGGCGTGTGGGGCCAAGGCACC | |
| | | CTGGTCACCGTCTCTTCAGGGCAACCTAAGGCT | |
| | Light | ACAGATGCCAGATGCGAGCTCGATCTGACCCAGACACCA | 301 |
| | | GCCTCCGTGTCTGAACCTGTGGGAGGCACAGTCACCATC | |
| | | AAGTGCCAGGCCAGTCAGAGCATTGGTAGTAGCTTAGCC | |
| | | TGGTATCAGCAGAAACCAGGGCAGCCTCCCAAGCTCCTG | |
| | | ATCTACAGGGCATCCACTCTGGCATCTGGGGTCCCATCG | |
| | | CGGTTCAGTGGCAGTGGATCTGGGACAGAGTTCACTCTC | |
| | | ACCATCAGCGACCTGGAGTGTGCCCATGCTGCCACTTAC | |
| | | TACTGTCAAAACTATATTAGTAGTAGTACTTTTGGGAAT | |
| | | GATTTCGGCGGAGGAACCGAGGTGGTGGTCAAAGGTGAT | |
| | | CCAGTTGCAC | |
| R-21 | Heavy | ACAGGTGTCCACTCGCAGTCGGTGGAGGAGTCCGGGGGT | 302 |
| | | CGCCTGGTCACGCCTGGGACACCCCTGACACTCACCTGC | |
| | | ACAGTCTCTGGAATCGACCTCAGTAGCTATGCAATCAAC | |
| | | TGGGTCCGCCAGGCTCCAGGGGAGGGGCTGGAATGGATC | |
| | | GGAATGATTAGTAGTAGTGATAACACATACTACGCGAAC | |
| | | TGGGCGAAAGGCCGATTCACCATCTCCAGAGACTCGACC | |
| | | ACGGTGGATCTGAAAATCACCAGTCCGACAACCGAGGAC | |
| | | ACGGCCACCTATTTCTGTGCCAGATTTTACGCTGGTAGA | |
| | | AGTTATTCCTTGTGGGGCCAAGGCACCCTGGTCACCATC | |
| | | TCCTCAGGGCAACCTAAGGCT | |
| | Light | ACAGATGCCAGATGCGAGCTCGATCTGACCCAGACACCA | 303 |
| | | GCCTCCGTGTCTGCAGCTGTGGGAGGCACAGTCACCATC | |
| | | AGTTGCCAGTCCAGTGAGAGTGTTTATAAGAGCAACTAT | |
| | | TTATCTTGGTATCAGCAAAAAGCAGGGCAGCCTCCCAAG | |
| | | CTCCTAATCTATGCTGCAGCCACTCTGGCATCTGGGGTC | |
| | | CCATCGCGGTTCAAAGGCAGTGGATCTGGGACACAGTTC | |
| | | ACTCTCACCATCAGCGGCGTGCAGTGTGACGATGCTGCC | |
| | | ACTTACTACTGTCAAGGCACTTATTATAGTAGTGGTTGG | |
| | | TACAATGCTTTCGGCGGAGGGACCGAGGTGGTGGTCAAA | |
| | | GGTGATCCAGTTGCA | |
| R-22 | Heavy | ACAGGTGTCCACTCGGAGCAGTCGGTGGAGGAGTCCGGG | 304 |
| | | GGTCGCCTGGTCACGCCTGGGACACCCCTGACACTCACC | |
| | | TGCACAGCCTCTGGATTCTCCCTCAGTACTTACTACATG | |
| | | AGTTGGGTCCGCCAGGCTCCAGGGAAGGGGCTGGAATGG | |
| | | ATCGGGATCATTCGTGGTGGTGGTGAACCTGTATGGTAC | |
| | | GCGGACTGGGCAAAAGGCCGATTCACCATCTCCAGAACC | |
| | | TCGACCACGGTGGATCTGAAAATCACCAGTCCGACGCCC | |
| | | GAGGACACGGCCACCTATTTCTGTGCCAGAAGGCTGGAT | |
| | | GGTTTTGTTAGGGCTTATCTGGACTTGTGGGGCCAGGGC | |
| | | ACCCTGGTCACCGTCTCTTCAGGGCAACCTAAGGCT | |
| | Light | ACAGATGCCAGATGCACCCAGACACCAGCCTCCGTGGAG | 305 |
| | | GCAGCTGTGGGAGGCACAGTCACCATCAAATGCCAGGCC | |
| | | AGTGAGAGTATTAGTAGTTGGTTGGCCTGGTATCAGCAG | |
| | | AAACCAGGGCAGCCTCCCAAGCTCCTGATCTATGAAGCA | |
| | | TTCAGTCTGGCCTCTGGGGTCCCATCGCGGTTCAGCGGC | |
| | | AGTGGATCTGGGACAGAGTTCACTCTCACCATCAGCGAC | |
| | | CTGGAGTGTGACGATGCTGCCATTTATTACTGTCAAAGT | |
| | | GCTTATTATGGTGGTACTTATGTTGGAGGTTTCGGCGGA | |
| | | GGGACCGAGGTGGTGGTCAAAGGTGATCCAGTTGCATCC | |
| | | AGTTGCA | |
| R-23 | Heavy | ACAGGTGTCCACTCGGAGCAGTCGGTGGAGGAGTCCGGG | 306 |
| | | GGTCGTCTGGTCATGCCTGGAGGATCCCTGACACTCACC | |
| | | TGCACAGTCTCTGGAATCGACCTCAGTAGCAATACAATG | |
| | | GGCTGGTTCCGCCAGGCTCCAGGGAAGGGGCTGGAATGG | |
| | | ATCGGGATTATGCGTGGTGGTGGTAGCACATACTACGCG | |
| | | ACCTGGGCGAAAGGCCGATTCACCATCTCCAAAACCTCG | |
| | | ACCACGGTGGATCTGAAACTCACCAGTCCGACAACCGAG | |
| | | GACACGGCCACCTATTTCTGTGCCAGAGCCTTCCCTGGT | |
| | | TATAATTCTTTCTTTAACTTCTGGGGCCAAGGCACCCTG | |
| | | GTCACCATCTCTTCAGGGCAACCTAAGGCT | |
| | Light | ACAGATGCCAGATGCGAGCTCGATCTGACCCAGACACCA | 307 |
| | | GCCTCCGTGTCTGAACCTGTGGGAGGCACAGTCACCATC | |
| | | AAGTGCCAGGCCAGTCACAACATTAATAGTGCTTTGGCC | |
| | | TGGTATCAGCAGAAACCAGGGCAGCCTCCCAAGCTCCTG | |
| | | ATCTACAAGGCATCCACTCTGGCATCTGGGGTCCCACCG | |
| | | CGGTTCAAAGGCAGTGGATCTGGGACAGAGTTCACTCTC | |
| | | ACCATCAGCGACCTGGAGTGTGCCGATGCTGCCACTTAC | |
| | | TACTGTCAAAGGCATTATTTCGATGGTGCCAGTGCTGAT | |
| | | ATAGCTTTCGGCGGAGGGACCGAGGTGGTGGTCAAAGGT | |
| | | GATCCAGTTGCA | |
| R-25 | Heavy | ACAGGTGTCCACTCGGCAGCGGTGAAGGAGTCCGGGGGT | 308 |
| | | CGCCTGGTCACGCCTGGGACACCCCTGACACTCACCTGC | |
| | | ACAGTCTCTGGATTCTCCCTCAGTAACTATGCAATGGGG | |
| | | TGGGTCCGCCAGCCTCCAGGGAAGGGGCTGGAGTGGATC | |
| | | GGATTCAGTGGTTATCCTGGTCGCACATACTATGCGAGT | |
| | | TGGGCGAAAGGCCGATTCACCATCTCCAAAACCTCGACC | |
| | | ACGGTGGATCTGAAAATCACCAGTCCGACAGCCGAGGAC | |
| | | ACGGCCACCTATTTCTGCGCCAGACATTATGGTAGCACT | |
| | | AACGGGCTTAACTTGTGGGGCCAAGGCACCCTGGTCACC | |
| | | GTCTCTTCAGGGCAACCTAAGGCT | |
| | Light | ACAGATGCCAGATGCGAGCTCGATCTGACCCAGACACCA | 309 |
| | | GCCTCCGTGTCTGAACCTGTGGGAGGCACAGTCACCATC | |
| | | AAGTGCCAGGCCAGTCAGAACAATAATAACTACTTAGCC | |
| | | TGGTATCAACAGAAACCAGGGCAGCCTCCCAAGCTCCTG | |
| | | ATCTATGCTGCATCCAGTCTGGCATCTGGGGTCCCATCG | |
| | | CGGTTCAAAGGCAGTGGATCTGGGACACAGTTCACTCTC | |
| | | ACCATCAGCGACCTGGAGTGTGACGATGCTGCCACTTAC | |
| | | TACTGTCAATGTACTGATTATGGTCCTACTTATCTTGGT | |
| | | ACTTTCGGCGGAGGGACCGAGGTGGAAATCAAAGGTGAT | |
| | | CCAGTTGCA | |
| R-26 | Heavy | ACAGGTGTCCACTCGCAGTCGTTGGAGGAGTCCGGGGGA | 310 |
| | | GACCTGGTCAAGCCTGGGGCATCCCTGACACTCACCTGC | |
| | | ACAGCCTCTGGATTCTCCTTCAGTAGCTACTACGACATG | |
| | | TGCTGGGTCCGCCAGGCTCCAGGGAAGGGGCTGGAGTGG | |
| | | ATCGCATGTATTTATATTGGTAATAGTGGTAACACTTAC | |
| | | TACGCGAGCTGGGCGAAAGGCCGATTCACCATCTCCAGA | |
| | | ACCTCGTCGACCACGGTGGATCTGAAAATGTCCAGTCTG | |
| | | ACAGCGGCGGACACGGCCAGTTATTTCTGTGCGAGAGGT | |
| | | GCTAGTGGTTATAGTGGTTATGGTTGGTTTATTTTGTGG | |
| | | GGCCCAGGCACTCTGGTCACCATCTCTTCAGGGCAACCT | |
| | | AAGGCT | |
| | Light | ACAGATGCCAGATGCGAGCTCGTGATGACCCAGACTCCA | 311 |
| | | GCCTCCGTGTCTGCAGCTGTGGGAGGCACAGTCACCATC | |
| | | AAGTGCCAGGCCAGTCAGAGCATTGGTAGTAATTTGGCC | |
| | | TGGTATCAGCAGAAACCAGGGCAGCGTCCCAAACTCCTG | |
| | | ATCTATGATGCATCTGATCTGGCATCTGGGGTCCCATCG | |
| | | CGGTTCAAAGGCAGTGGTTCTGGGCCAGAGTACACCCTC | |
| | | ACCATCAGCGGCGTGGAGTGTGACGATGCTGCCACTTAC | |
| | | TACTGTCAACAGGGTGATACTTATATTAATGTTGATAAT | |
| | | ATTTTCGGCGGAGGGACCGAGGTGGAAATCAAAGGTGAT | |
| | | CCAGTTGCA | |
| R-28 | Heavy | ACAGGTGTCCACTCGGAGCAGTCGGTGGAGGAGTCCGGG | 312 |
| | | GGTCGCCTGGTAACGCCTGGGACACCCCTGACACTCACC | |
| | | TGCACAGTCTCTGGAATTGACCTCAGTAACTATATAATG | |
| | | GGCTGGGTCCGCCAGGCTCCAGGGAAGGGGCTGGAATGG | |
| | | ATCGGAATCATTGCTAGTAATGGTGGCACAGTCTACGCG | |
| | | AACTGGGCGAAAGGCCGATTCACCATCTCCAAAACCTCG | |
| | | ACCACGGTGGATCTGAAAATGACCAGTCCGACACCCGAG | |
| | | GACACGGCCACCTATTTCTGTGCCAGAGGTGCTGGTACT | |
| | | ACTTGGTCCTCCTTTAACTTGTGGGGCCAAGGCACCCTG | |
| | | GTCACCATCTCTTCAGGGCAACCTAAGGCT | |
| | Light | ACAGATGCCAGATGCGAGCTCGTGCTGACCCAGACACCA | 313 |
| | | GCCTCTGTGTCTGAACCTGTGGGCGGCACAGTCACCATC | |
| | | AATTGCCAGGTCAGTCAGAGCATTGATAACTACTTGAAC | |
| | | TGGTATCACCAGAAACCAGGGCAGCCTCCCAAGCGCCTG | |
| | | ATCTACAGGGCATCCACTCTGGCATCTGGGGTCCCATCG | |
| | | CGGTTCAGAGGCAGTGGATCTGGGACACAGTTCACTCTC | |
| | | ACCATCAGCGACCTGGAGTGTGCCGATGCTGCCACTTAC | |
| | | TACTGTCAAAGCTATTATGGTAGTAGTACTAATGCTTTC | |
| | | GGCGGAGGGACCGAGGTGGTGGTCAAAGGTGATCCAGT | |
| R-29 | Heavy | ACAGGTGTCCACTCGGAGCAGTCGGTGGAGGAGTCCGGG | 314 |
| | | GGTCGCCTGGTCACGCCTGGGACACCCCTGACACTCACC | |
| | | TGCACAGTCTCTGGATTCTCCCTCAGTAGTTATGCAATG | |
| | | AACTGGGTCCGCCAGGCTCCAGGGAAGGGACTGGAATGG | |
| | | ATCGGACTCATTTATGCTAGTGGTACCACTCACTACGCG | |
| | | AGCTGGGTGAATGGCCGATTCACCATCTCCAAAACCTCG | |
| | | ACCACGGTGACTCTGAAAATGACCAGTCTGACAGCCGCG | |
| | | GACACGGCCACCTATTTCTGTGCCAGAGATAGTTATAGT | |
| | | ACTGGTAACTTGTGGGGCCAAGGCACCCTGGTCACCATC | |
| | | TCTTCAGGGCAACCTAAGGCT | |
| | Light | ACAGATGCCAGATGCGAGCTCGTGCTGACCCAGACACCA | 315 |
| | | GCCTCCGTGTCTGAACCTGTGGGAGGCACAGTCACCATC | |
| | | AAGTGCCAGGCCAGTCAGAGCATTTACAGCAGTTTAGTC | |
| | | TGGTATCAGCAGAAACCAGGGCAGCCTCCCAAGCTCCTG | |
| | | ATCTATGGTGCGTCCAATCTGGAATCTGGGGTCCCATCG | |
| | | CGGTTCAAAGGCAGTGGATCTGGGACAGAGTACACTCTC | |
| | | ACCATCAGCGGCGTGCAGTGTGCCGATGCTGCCACTTAC | |
| | | TACTGTCAATGTAGTGGTTATAATAGTCGTGTTTTCGGC | |
| | | GGAGGGACCGAGGTGGAAATCAAAGGTGATCCAGTTGCA | |
| R-30 | Heavy | ACAGGTGTCCACTCGAGCAGTTCGGTGGAGGAGTCCGGG | 316 |
| | | GGAGGCCTGGTCAAGCCTGGGGCATCCCTGACACTCACC | |
| | | TGCACAGCCTCTGGATTCTCCTTCAGTAGCGGCTACTGG | |
| | | ATGTGCTGGGTCCGCCAGGCTCCAGGGAAGGAACTGGAG | |
| | | TGGATCGCATGCATTGGCGCTCGTGATAGTGGTAGCGCT | |
| | | TACTACGCGACCTGGGCGAAAGGCCGATTCACCATCTCC | |
| | | AAAATCTCGTCGACCACGGTGACTCTGCAAATGACCAGT | |
| | | CTGACAGCCGCGGACACGGCCACCTATTTCTGTGCGAGA | |
| | | TCTGGTGCTAGTTTCCACTTTAAATTGTGGGGCCCAGGC | |
| | | ACCCTGGTCACCATCTCTTCAGGGCAACCTAAGGCT | |
| | Light | ACAGATGCCAGATGCGAGCTCGTGCTGACCCAGACACCA | 317 |
| | | GCCTCCGTGTCTGAACCTGTGGGAGGCACAGTCACCATC | |
| | | AAGTGCCAGGCCAGTCAGAACATCGGTAGCTACTTAGCC | |
| | | TGGTATCAGCAGAAACCAGGGCAGCCTCCCAAGCTCCTG | |
| | | ATCTATGGTGCGTCCAATCTGGAATCTGGGGTCCCATCG | |
| | | CGGTTCAAAGGCAGTGGATCTGGGACAGAGTACACTCTC | |
| | | ACCATCAGCGGCGTGCAGTGTGCCGATGCTGCCACTTAC | |
| | | TACTGTCAATGTAGTGGTTATAATAGTCGTGTTTTCGGC | |
| | | GGAGGGACCGAGGTGGTGGTCAAAGGTGATCCAGTTGCA | |
| R-31 | Heavy | ACAGGTGTCCACTCGCAGTCGCTGGAGGAGTCCGGGGGA | 318 |
| | | GACCTGGTCACGCCTGGGACACCCCTGACACTCACCTGC | |
| | | ACAGTCTCTGGATTCTCCCTCAGTACCTACAACATTCAA | |
| | | TGGGTCCGCCAGGCTCCAGGGAAGGGTCTGGAATGCATC | |
| | | GGATTCATTAATGGTGGTGGTAGCGCATACTACGCGAGC | |
| | | TGGGTGAATGGTCGATTCACCGTCTCCAAAGCCTCGACC | |
| | | ACGGTGACTCTGAAAATCACCGCTCCGACAACCGAGGAC | |
| | | ACGGCCACCTATTTCTGTGCCAGAGGTTATTATGCTGGT | |
| | | GCTGATTGGAATATGCCGTTGGATCTCTGGGGCCAGGGC | |
| | | ACGCTGGTCACCGTCTCTTCAGGGCAACCTAAGGCT | |
| | Light | ACAGATGCCAGATGCGAGCTCGTGCTGACCCAGACACCT | 319 |
| | | TCGCCCGTGTCTGCAGGTGTGGGAGGCACAGTCACCATC | |
| | | AAGTGCCAGTCCAGTGAGAGCATTGGCACTGCATTAGCC | |
| | | TGGTATCAGCAGAAACTAGGGCAGCCTCCCAAGCTCCTG | |
| | | ATCTATGCTGCAGCCCTTCTGGCATCTGGGGTCTCATCG | |
| | | CGGTTCAAAGGCAGCGGATCTGGGACAGAGTACACTCTC | |
| | | ACCATCAGCGACCTGGAGTGTGACGATGCTGCCACTTAC | |
| | | TACTGTCAATTTACTTATTATGGTAATAGTTATGGGAAT | |
| | | GCTTTCGGCGGAGGGACCGAGGTGGAAATCAAAGGTGAT | |
| | | CCAGTTGCA | |
| R-32 | Heavy | ACAGGTGTCCACTCGCAGTCGGTGAAGGAGTCCGGGGGA | 320 |
| | | GACCTGGTCAAGCCTGAGGGATCCCTGACACTCACCTGC | |
| | | ACAGCCTCTGGATTCTCCTTCAGTGGCGGCTACGACATG | |
| | | TGCTGGGTCCGCCAGGCTCCAGGGAAGGGGCTGGAGTGG | |
| | | ATCGCATGCATTTATGCTGGTAGTAGTGGTAGCACTTAC | |
| | | TACGCGAGCTGGGCGAAAGGCCGATTCACCATCTCCAAA | |
| | | ACCTCGTCGACCACGGTGACTCTGCAAATGACCAGTCTG | |
| | | ACAGCCGCGGACACGGCCACCTATTTCTGTGCGAGAGGG | |
| | | GCTTATGCTGGTGGTGGTTACCCCCCCTACTTTAATTTC | |
| | | TGGGGCCCAGGCACCCTGGTCACCATCTCTTCAGGGCAA | |
| | | CCTAAGGCT | |
| | Light | ACAGATGCCAGATGCGAGCTCGTGATGACCCAGACACCA | 321 |
| | | GCCTCCGTGGAGGCAGCTGTGGGAGGCACAGTCACCATC | |
| | | AATTGCCAGGCCAGTGAGAGCATTAACGATAGATTAGCC | |
| | | TGGTATCAGCTGAAACCAGGGCAGCCTCCCAAACTCCTG | |
| | | ATCTACGAAACATCCAAAGTGGAATCTGGGGTCCCATCG | |
| | | CGGTTCAGCGGCAGTGGATCTGGGACACAGTTCACTCTC | |
| | | ACCATCAGCGACCTGGAGTGTGCCGATGCTGCCACTTAC | |
| | | TACTGTCAAAGCCATGATTATATTAGTAGTAGTGGTATG | |
| | | CCTTTCGGCGGAGGGACCGAGCTGGAAATCAAAGGTGAT | |
| | | CCAGTTGCA | |
| R-34 | Heavy | ACAGGTGTCCACTCGGAGCAGTCGGTGAAGGAGTCCGGG | 322 |
| | | GGTCGCCTGGTCACGCCTGGGACACCCCTAACACTCACC | |
| | | TGCACAGTCTCTGGATTCTCCCTCAGTAGCAGTGGAATG | |
| | | AGCTGGGTCCGCCAGGCTCCAGGGAAGGGGCTGGAATGG | |
| | | ATCGGACTCACTTTTCCTAGTGGTAGCGCATCCTACGCG | |
| | | AGCTGGGCGAAAGGCCGATTTACCGTCTCCAGGACCTCG | |
| | | ACCACGGTGGATCTGAATCTCACCAGTCCGACAACCGAA | |
| | | GACACGGCCACCTATTTCTGTGCCCATGTTGGTGCTACT | |
| | | ACTGGTCGTGACTTCTGGGGCCAAGGCACCCTGGTCACC | |
| | | GTCTCCTCAGGGCAACCTAAGGCT | |
| | Light | ACAGATGCCAGATGCGAGCTCGTGATGACCCAGACTGCA | 323 |
| | | TCGTCCGTGTCTGCAGCTGTGGGAGGCACAGTCACCATC | |
| | | AGTTGCCAGTCCAGTCAGAATATTTATAACAACCGCTGG | |
| | | TCCTGGTATCAGCAGAAACCAGGGCAGCCTCCCAAGCTC | |
| | | CTGATCTACCAGGCATCCACTCTGGCATCTGGGGTCCCA | |
| | | TCGCGGTTCAAAGGCAGTGGATCTGGGACACAGTTCACT | |
| | | CTCACCATCAGCGGCGTGCAGTGTGACGATGCTGCCACT | |
| | | TACTACTGTGCAGGCGGTTATAGTGGTACGATTTTTACT | |
| | | TTCGGCGGAGGGACCGAGCTGGAAATCAAAGGTGATCCA | |
| | | GTTGCA | |
| R-35 | Heavy | ACAGGTGTCCACTCGGCAGCGGTGAAGGAGTCCGGGGGT | 324 |
| | | CGCCTGGTCACGCCTGGGACACCCCTGACACTCACCTGC | |
| | | ACAGTCTCTGGAATCGACCTCAGTGACTACTACATGACC | |
| | | TGGGTCCGCCAGGCTCCAGGGAAGGGGCTGGAATACATC | |
| | | GGAATCATTTATGCTAGTGGCAACGCATACTTTGCGAGT | |
| | | TGGGCGAAAGGCCGATTCACCATCTCCAGAACCTCGTCG | |
| | | ACCACGGTGGATCTGAATGTCGCCAGTCCGACAACCGAG | |
| | | GACACGGCCACCTATTTTTGTGCCAGGAATATTGGTGGT | |
| | | AGTAGTAACTTGTGGGGCCAAGGCACCCTGGTCACCGTC | |
| | | TCTTCAGGGCAACCTAAGGCT | |
| | Light | ACAGATGCCAGATGCGAGCTCGATATGACCCAGACACCA | 325 |
| | | GCCTCCGTGTCTGAACCTGTGGGAGGCACAGTCACCATC | |
| | | AAGTGCCAGGCCAGTGAGAGAATTTATAATAATTTAGCC | |
| | | TGGTATCAGCAGAAACCAGGGCAGCCTCCCAAACTCCTC | |
| | | TTCTATTCTGCATCCACTCTGGCATCTGGGGTCCCTTCG | |
| | | CGATTCAAAGGCAGTGGATCTGGGACAGAGTTCACTCTC | |
| | | ACCATCAGCGACCTGGAGTGTGCCGATGCAGCCATTTAC | |
| | | TACTGTCAATGTACTGGTTATGGTACTACCTATGGGCTT | |
| | | GTTTTCGGCGGAGGGACCGAGGTGGAAATCAAAGGTGAT | |
| | | CCAGTTGCA | |
| R-36 | Heavy | ACAGGTGTCCACTCGCAGTCGGTGAAGGAGTCCGGGGGA | 326 |
| | | GGCCTGTTCCAGCCTGGGGGATCCCTGGCACTCACCTGC | |
| | | AAAGCCTCTGGATTCTCCCTCAGTAAAAATTACGTGATG | |
| | | TGCTGGGTCCGCCAGGCTCCAGGGAAGGGGCTGGAGTGG | |
| | | ATCGCATGCATTTATGCTGGTGATGGTAACACTTACTAC | |
| | | GTGAGCTGGGCGAAAGGCCGATTCACCATCTCCAAAACC | |
| | | TCGTCGACCACGGTGACTCTGCAAATGACCAGTCTGACA | |
| | | GCCGCGGACACGGCCACCTATTTCTGTGCGAGATGGGAC | |
| | | TATATTGGTAATAATAATTTGTGGGGCCCAGGCACCCTG | |
| | | GTCACCATCTCCTCAGGGCAACCTAAGGCT | |
| | Light | ACAGATGCCAGATGCGAGCTCGTGCTGACCCAGACACCA | 327 |
| | | GCCTCCGTGGAGGCAGCTGTGGGAGGCACAGTCACCATC | |
| | | AAGTGCCAGGCCAGTCAGACCATTAGTAGCTACTTAGCC | |
| | | TGGTATCAGCAGAAACCAGGGCAGCCTCCCAAGCTCCTG | |
| | | ATCTACAGGGCATCCACTCTGGCATCTGGGGTCCCATCG | |
| | | CGGTTCAAAGGCAGTGGATCTGGGACACAGTTCACTCTC | |
| | | ACCATCAGCGACCTGGAGTGTGCCGATGGTGCCACTTAC | |
| | | TACTGTCAATGTCCTTATTATGGTAGTAGCCATGTGGGT | |
| | | GCTTTCGGCGGAGGGACCGAGGTGGTGGTCAAAGGTGAT | |
| | | CCAGTTGCA | |
| R-37 | Heavy | ACAGGTGTCCACTCGCAGTCGGTGAAGGAGTCCGGGGGT | 328 |
| | | CGCCTGGTCACGCCTGGGACACCCCTGACACTCACCTGC | |
| | | ACAGTCTCTGGAATCGACCTCATTAGCTATCCAATGGGC | |
| | | TGGGTCCGCCAGGCTCCAGGGAAGGGGCTGGAATACATC | |
| | | GGAATCATTGCTACTGGTGGTAGCACATACTACGCGAGC | |
| | | TGGGCAAAAGGCCGATTCACCATCTCCACAAGCTCGACC | |
| | | ACGGTGGATCTGAAAATGACCAGTCTGACAGCCGCGGAC | |
| | | ACGGCCACCTACTTCTGTGCCAGAAGATGGAGTAGTGCT | |
| | | TATGACTTCTGGGGCCAAGGCACCCTGGTCACCATCTCT | |
| | | TCAGGGCAACCTAAGGCT | |
| | Light | ACAGATGCCAGATGCGAGCTCGATCTGACCCAGACACCA | 329 |
| | | GCCTCTGTGGAGGTAGCTGTGGGAGGCACAGTCACCATC | |
| | | AAGTGCCAGGCCAGTCAGAGCATTAGTAGCTACTTAGCC | |
| | | TGGTATCAGCAGAAACCAGGGCAGCCTCCCAAGCTCCTG | |
| | | ATCTATTATGCATCCACTCTGGCATCTGGGGTCCCATCG | |
| | | CGGTTCAGCGGCAGTGGATCTGGGACAGAGTTCACTCTC | |
| | | ACCATCAGTGGCGTGCAGTGTGACGATGCTGCCACTTAC | |
| | | TACTGTCAACAGGGTGCTAGAAGTAGTAATGTTGATAAT | |
| | | ATTTTCGGCGGAGGGACCGAGGTGGTGGTCAAAGGTGAT | |
| | | CCAGTTGCA | |
| R-39 | Heavy | ACAGGTGTCCACTCGCAGTCGTTGGAGGAGTCCGGGGGT | 330 |
| | | CGCCTGGTAACGCCTGGAGGATCCCTGACACTCACCTGC | |
| | | ACAGTCTCTGGAATCGACCTCAGTAGTTATACAATGGGC | |
| | | TGGGTCCGCCAGGCTCCAGGGAAGGGGCTGGAATGGATC | |
| | | GGAGTCATTGCTATTAATGGTGGCACAGTCTACGCGAGC | |
| | | TGGGCGAAAGGCCGATTCACCATCTCCAAAACCTCGACC | |
| | | ACGGTGGATCTGAAAATGACCAGTCCGACACCCGAGGAC | |
| | | ACGGCCACCTATTTCTGTGCCAGAGGTGCTGGTAAAACT | |
| | | TGGTCCTCCTTTAACTTGTGGGGCCAAGGCACCCTGGTC | |
| | | ACCATCTCCTCAGGGCAACCTAAGGCT | |
| | Light | ACAGATGCCAGATGCGAGCTCGTGCTGACCCAGACACCA | 331 |
| | | GCCTCCGTGTCTGAACCTGTGGGAGGCACAGTCACCATC | |
| | | AAGTGCCAGGCCAGTCAGAGCATTGGTAGTAATTTAGCC | |
| | | TGGTATCAGCAGAAACCAGGGCAGCCTCCCAAGCTCCTG | |
| | | ATCTACAGGGCATCCACTCTGGAATCTGGGGTCCCATCG | |
| | | CGGTTCAGCGGCAGTGGATCTGGGACAGAGTTCACTCTC | |
| | | ACCATCAGCGACCTGGAGTGTGCCGATGCTGCCACTTAC | |
| | | TACTGTCAAAGCTATTATGGTAGTAGTACTAATGCTTTC | |
| | | GGCGGAGGGACCGAGGTGGTGGTCAAAGGTGATCCAGTT | |
| | | GCA | |
| R-40 | Heavy | ACAGGTGTCCACTCGCAGTCGGTGAAGGAGTCCGGGGGT | 332 |
| | | CGCCTGGTCAAGCCTGACGAAACCCTGACAATCACCTGC | |
| | | ACAGTGTCTGGAATCGACCTCAGTCGCTACAGCATACAG | |
| | | TGGGTCCGCCAGGCTCCAGGGGGGGGGCTGCAATGGATC | |
| | | GGTGTCATGAGTATCGGTAGTAGCGCATACTACGCGAAC | |
| | | TGGGCGAAAGGCCGATTCACCATCTCCAAAACCTCGTCG | |
| | | ACCACGGTGGATCTGACAATGACCAGTCTGACAGCCGCG | |
| | | GACACGGCCACCTATTTCTGTGCCAGAAGTAATCTCTAC | |
| | | CGGGGGGCTTTTGATCCCTGGGGCCCAGGCACCCTGGTC | |
| | | ACCATCTCTTCAGGGCAACCTAAGGCT | |
| | Light | ACAGATGCCAGATGCGAGCTCGTGATGACCCAGACACCA | 333 |
| | | GCCTCCGTGGAGGCAGCTGTGGGAGGCACAGTCACCATC | |
| | | AAGTGCCAGTCCAGTGAGAGTGTTTATAAGAATAACTAC | |
| | | TTATCCTGGTATCAACTTAAACCAGGGCAGCCTCCCAAA | |
| | | CTCCTCTTCTATTCTGCTTCCACTCTGGCATCTAGGGTC | |
| | | CCATCGCGGTTCAGCGGCAGTGGATCTGGGACACAGTTC | |
| | | ACTCTCACCATCAGCGACCTGGAGTGTGACGATGCTGCC | |
| | | ACTTACTACTGTGCAGGGTATTATGGTAGTAGTAGTGIT | |
| | | GATCGTTACACTTTCGGCGGAGGGACCGAGGTGGAAATC | |
| | | AAAGGTGATCCAGTTGCAC | |
| R-41 | Heavy | ACAGGTGTCCACTCGGAGCAGCTGATGGAGTCCGGGGGA | 334 |
| | | GGCCTGGTCCAGCCTGGTGCATCCCTGACACTCACCTGC | |
| | | AAAGCCTCTGGATTCTCCTTCAGTAGCAGCCAGGACATG | |
| | | TGCTGGGTCCGCCAGGCTCCAGGGAAGGGGCCTGAGTGG | |
| | | ATCGGATGCATTGGTTTTGGTAGTGGTAACACATGGTAC | |
| | | GCGAGCTGGGTGAATGGCCGATTCACCATCTCCAGAAGC | |
| | | ACCAGCCTAAACACGGTGGAGCTGAAAATGACCGGTCTG | |
| | | ACAGCCGCGGACACGGCCACCTATTTCTGTGCGAGAGAT | |
| | | CCATTTACTGTTGCTGGTGATGGTGATAATTTGTGGGGC | |
| | | CCAGGCACCCTGGTCACCGTCTCCTCAGGGCAACCTAAG | |
| | | GCT | |
| | Light | ACAGATGCCAGATGCGAGCTCGATCTGACCCAGACACCA | 335 |
| | | GCCTCCGTGTCTGAACCTGTGGGAGGCACAGTCACCATC | |
| | | AAGTGCCAGGCCAGTCAGACCATTAACAATGCATTAGCC | |
| | | TGGTATCAGCAGAAACCAGGGCAGCCTCCCAAGCTCCTG | |
| | | ATCTATTCTGCATCCACTCTGGCATCTGGGGTCTCATCG | |
| | | CGGTTCAGCGGCAGTGGATCAGGGACACAGTTCACTCTC | |
| | | ACCATCAGTGGCGTGGAGTGTGACGATGCTGCCACTTAC | |
| | | TACTGTGCAGGCTATAGACCTTATATTAATGATGGTAGT | |
| | | GCTTTCGGCGGAGGGACCGAGGTGGTGGTCAAAGGTGAT | |
| | | CCAGTTGCA | |
| R-42 | Heavy | ACAGGTGTCCACTCGGAGCAGTCGGTGGAGGAGTCCGGG | 336 |
| | | GGTCGCCTGGTCACGCCTGGGACACCCCTGACACTCACC | |
| | | TGCACAGTCTCTGGATTCTCCCTCAGTAGTTATGCAATG | |
| | | AGCTGGGTCCGCCAGGCTCCAGGGAAGGGGCTGGAGTGG | |
| | | ATCGGAGTCATTGATGTTACTGGTAGGACATACTACGCG | |
| | | AGCTGGGCGAAAGGCCGATTCACCATCTCCAAAACCTCG | |
| | | ACCACGGTGGATCTGAAAATCACCAGTCCGACAACCGAG | |
| | | GACACGGCCACCTATTTCTGTGGCAGAGATAGCTACCTT | |
| | | GACTATGGTGACCCCAATGGTCTTGATCCCTGGGGCCCA | |
| | | GGCACCCTGGTCACCATCTCCTCAGGGCAACCTAAGGCT | |
| | Light | ACAGATGCCAGATGCGAGCTCGTGATGACCCAGACACCA | 337 |
| | | GCCTCCGTGGAGGCAGCTGTGGGAGGCACAGTCACCATC | |
| | | AAGTGCCAGGCCAGTCAGAGCATTGGTAGTAATTTAGCC | |
| | | TGGTATCAGCAGAAACCAGGGCAGCCTCCCAAGCTCCTG | |
| | | ATCTATACTGCATCCAATCTGGCGCCTGGAGTCTCATCG | |
| | | CGGTTCAAAGGCAGTAGATCTGGGACAGAGTCCACTCTC | |
| | | ATCATCAGCGACCTGGAGTGTGCCGATGCTGCCACTTAC | |
| | | TACTGTCAATGTACTTATTATGGTACTAGTTATATGGAA | |
| | | TTAGGGGCTTTCGGCGGAGGGACCGAGGTGGTGGTCAAA | |
| | | GGTGATCCAGTTGCAC | |
| R-45 | Heavy | ACAGGTGTCCACTCGCAGTCGGTGAAGGAGTCCGGGGGT | 338 |
| | | CGCCTGGTCACGCCTGGGACACCCCTGACACTCACCTGC | |
| | | ACAGTCTCTGGATTCTCCATCAATGACTTCCACATGTGC | |
| | | TGGGTCCGCCAGGCTCCAGGGAAGGGGCTGGAATACATC | |
| | | GGAATGATTGGTACTGGTGGTAGCGCATACTACGCGACC | |
| | | TGGACAAAAGGCCGATTCACCATCTCCAGAACCTCGACC | |
| | | ACGGTGGATCTAAAGATCACCAGTCCGACAACCGAGGAC | |
| | | ACGGCCACCTATTTCTGTGCCAGACGGGATTGGAGTGAT | |
| | | GGTGGTTATTCGCTATTATGGGGCCAAGGCACCCTGGTC | |
| | | ACCGTCTCTTCAGGGCAACCTAAGGCT | |
| | Light | ACAGATGCCAGATGCGAGCTCGATCTGACCCAGACACCA | 339 |
| | | GCCTCCGTGTCTGAACCTGTGGGAGGCACAGTCACCATC | |
| | | AAGTGCCAGGCCAGTCAGAGCATTCGCACCTACTTAAAT | |
| | | TGGTATCAGCAGAAACCAGGGCAGCCTCCCAAACTCCTG | |
| | | ATCTATTCTGCATCCACTCTGGCATCTGGGATCCCATCG | |
| | | CGGTTCAAAGGCAGTGGATCTGGGACACAGTTCACTCTC | |
| | | ACCATCAGCGACCTGGAGTGTGCCGATGCTGCCACTTAT | |
| | | TACTGTCAACAGGGATATTATTGGAGTGATGTTGAGAAT | |
| | | ACTTTCGGCGGAGGGACCGAGGTGGAAATCAACGGTGAT | |
| | | CCAGTTGCA | |
| R-49 | Heavy | ACAGGTGTCCACTCGCAGTCGGTGAAGGAGTCCGGGGGT | 340 |
| | | CGCCTGGTCACGCCTGGGACACCCCTGACACTCACCTGC | |
| | | ACCGTCTCTGGATTCTCCCTCAGTATCAATGCAATAAAC | |
| | | TGGGTCCGCCAGGCTCCAGGGAAGGGGCTAGAATGGATC | |
| | | GGGATCATTACTAATATTGGTAACACATACTACGCGAGC | |
| | | TGGGCAAAAGGCCGATTCACCATCTCCAGAACCTCGACC | |
| | | ACGGTGGATCTGACAATGACCAGTCTGACAACCGAGGAC | |
| | | ACGGCCACCTATTTCTGTGCCAGAAACTCTTATTTTGAT | |
| | | CCCTGGGGCCCAGGCACCCTGGTCACCATCTCTTCAGGG | |
| | | CAACCTAAGGCT | |
| | Light | ACAGATGCCAGATGCGAGCTCGTGCTGACCCAGACACCA | 341 |
| | | TCCCCCGTGTCTGCAGCTGTGGGAGGCACAGTCAGCATC | |
| | | AGTTGCCAGGCCAGTCAAAGTGTTGATAGTAATAACTAC | |
| | | TGTTCCTGGTATCAGCAGAAACCAGGGCAGCCTCCCAAG | |
| | | CTCCTGATCCACAAGGCATCCAAACTGGCATCTGGGGTC | |
| | | CCATCGCGGTTCAAAGGCAGTGGATCTGGGACACAGTTC | |
| | | ACTCTCACCATCAACGACCTGGAGTGTGACGATGCTGCC | |
| | | ACTTACTACTGTGCAGGCGGTTTGGGTGATAATATTTAT | |
| | | GCTTTCGGCGGAGGGACCGAGGTGGAAATCAACGGTGAT | |
| | | CCAGTTGCA | |
| R-51 | Heavy | ACAGGTGTCCACTCGCAGTCGGTGAAGGAGTCCGGGGGT | 342 |
| | | CGCCTGGTCACGCCTGGGACACCCCTGACACTCACCTGC | |
| | | ACAGTCTCTACAATCGACGTCAGTAAGTATGCAGTGATC | |
| | | TGGGTCCGCCAGGCTCCAGGGAAGGGGCTGGAATACATC | |
| | | GGAATCATTAGTAGTAGTGGTAGCACATACTACGCGAGC | |
| | | TGGGCGAAAGGCCGATTCACCATCTCCAAAACCTCATCG | |
| | | ACCACGGTGGATCTGAAAATGACCAGTCCGACAGCCGAG | |
| | | GACACGGCCACCTATTTCTGTGCCAGAGGGGGTAATGGT | |
| | | GTCGCCTTTAAATTATGGGGCCAAGGCACCCTGGTCACC | |
| | | ATCTCCTCAGGGCAACCTAAGGCT | |
| | Light | ACAGATGCCAGATGCGAGCTCGATCTGACCCAGACACCA | 343 |
| | | GCCTCCGTGGAGGCAGCTGTGGGAGGCACAGTCACCATC | |
| | | AATTGCCAGGCCAGTCAGAGCATTTACAGCTCTTTAGCC | |
| | | TGGTATCAGCAGAAACCAGGGCAGCCTCCCAAACTCCTG | |
| | | ATCTACGAAACATCCAAAGTGGAATCTGGGGTCCCATCG | |
| | | CGGTTCAGCGGCAGTGGATCTGGGACACAGTTCACTCTC | |
| | | ACCATCAGCGACCTGGAGTGTGCCGATGCTGCCACTTAC | |
| | | TACTGTCAAAGCCATGATTATATTAGTAGTAGTGGTATG | |
| | | CCTTTCGGCGGAGGGACCGAGCTGGAGATCCTAGGTGAT | |
| | | CCAGTTGCA | |

TABLE 6
EC₅₀ of anti-CD5L monoclonal antibodies

| mAb Name | EC50 (μg/ml) |
|---|---|
| R-1 | 0.0598 |
| R-3 | 0.1827 |
| R-4 | 0.0713 |
| R-5 | 0.1482 |
| R-6 | 0.1871 |
| R-9 | 0.1355 |
| R-10 | 0.3073 |
| R-11 | 0.04738 |
| R-13 | 0.1158 |
| R-14 | 0.1173 |
| R-17 | 0.2929 |
| R-18 | 0.1900 |
| R-21 | 0.2100 |
| R-23 | 0.1395 |
| R-29 | 0.1608 |
| R-33 | 0.1512 |
| R-34 | 0.2202 |
| R-35 | 0.1691 |
| R-36 | 0.2777 |
| R-39 | 0.2463 |
| R-40 | 0.2105 |
| R-43 | 0.3009 |
| R-45 | 0.0788 |
| R-49 | 0.0077 |
| R-51 | 0.0144 |
| H-7 | 0.1095 |
| H-118 | 0.1360 |
| H-119 | 0.0068 |
| H-447 | 0.2500 |

TABLE 7
Binding affinity to human
CD5L determined using Octet
(96-Red) instrument and cross
reactivity to murine CD5L

| Antibody | Human CD5L |
|---|---|
| Name | binding KD (nM) |
| R-3 | 2.18 |
| R-40 | 1.74 |
| R-34 | 1.35 |
| R-13 | 1.72 |
| R-45 | 2.97 |
| R-35 | 0.78 |
| R-18 | 1.56 |
| H118 | 16.5 |
| H007 | 5.87 |
| H447 | 28.7 |

TABLE 8
Epitope groups of CD5L mAbs

| mAb Name | Epitope Bins |
|---|---|
| R-1 | Bin1 |
| R-3 | Bin1 |
| R-4 | Bin4 |
| R-6 | Bin1 |
| R-11 | Bin1 |
| R-13 | Bin3 |
| R-14 | Bin1 |
| R-21 | Bin1 |
| R-23 | Bin1 |
| R-34 | Bin2 |
| R-35 | Bin5 |
| R-40 | Bin1 |
| R-45 | Bin4 |
| R-49 | Bin6 |
| R-50 | Bin6 |
| R-51 | Bin6 |
| H007 | Bin7 |
| H118 | Bin6 |
| H447 | Bin8 |

### Example 6—CD5L Overexpression is Associated with Bevacizumab Resistance and Worse Overall Survival in Ovarian Cancer Patients

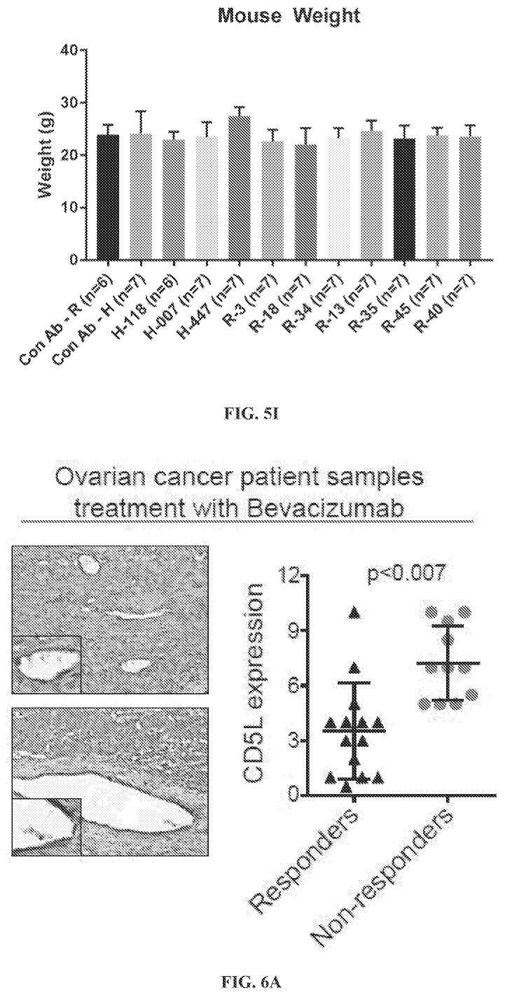

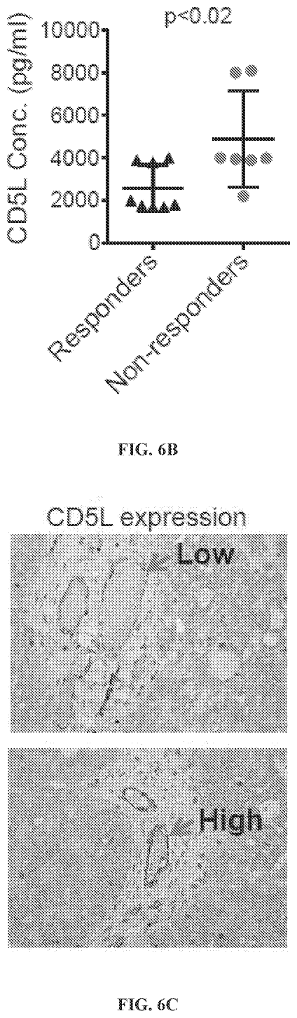

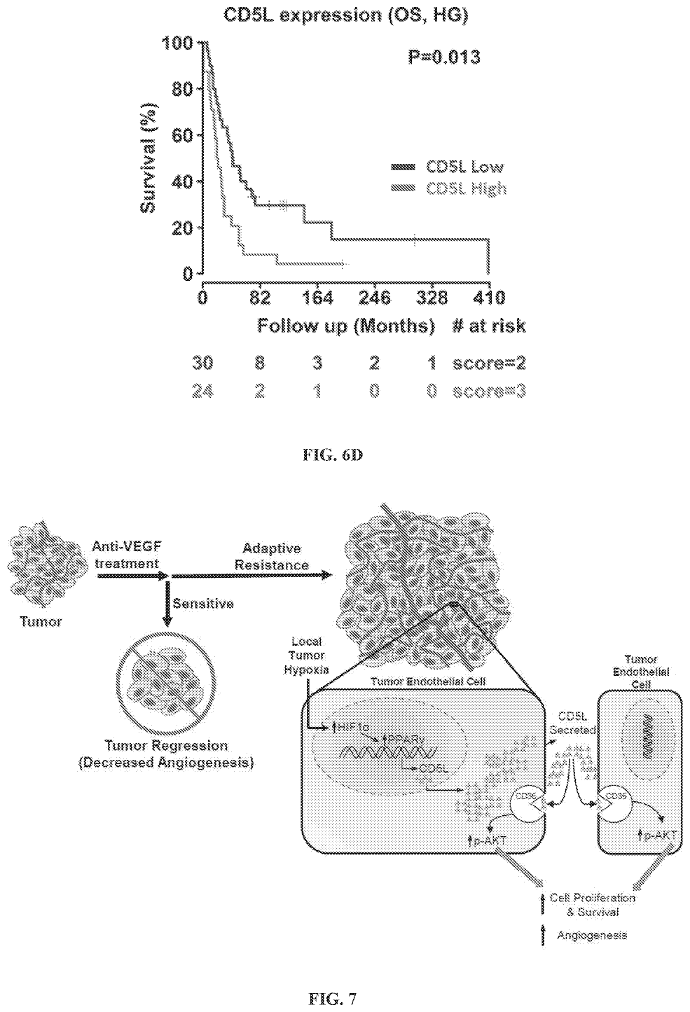

To determine potential clinical relevance of CD5L overexpression, a select cohort of ovarian cancer patients identified as bevacizumab-responders versus non-responders was interrogated. Patients with disease resistant to bevacizumab had significantly higher CD5L expression in their tumor endothelial cells compared to those with bevacizumab-sensitive disease (FIG. 6A). Furthermore, those patients who were bevacizumab resistant also had a significantly higher serum CD5L levels compared to patients sensitive to bevacizumab treatment (FIG. 6B). In addition, IHC staining for CD5L was performed on a tissue microarray (TMA) comprised of tumor samples from an ovarian cancer cohort. CD5L expression in tumor endothelial cells ranged from absent to low (FIG. 6C, upper) to high (FIG. 6C, lower) in this cohort. Interestingly, in ovarian cancer patients with high-grade serous histology, those with high tumor endothelial CD5L expression had significantly worse overall survival than those with low expression (FIG. 6D).

### Example 7—Effect of CD5L Antibody (R-35) on OVCAR8 Tumor Growth and Survival and Safety Studies

Experimental design. The inventors examined the effects of CD5L antibody (R-35) on normal C57/BL6 mice. The mice were treated with control IgG or CD5L Ab for two weeks (once a week for two weeks) and blood was collected at 24 hours post-injection; CBC (complete blood count) and other blood counts were assessed.

Tissue staining. H&E staining of vital organs such as lung, liver, kidney and spleen were obtained after treating mice with IgG control or CD5L antibody. Two in house pathologists examined the slides. CD31 staining was performed on these vital organ tissues to stain the blood vessels.

Survival Assay. The effect of CD5L inhibition alone or combined with bevacizumab on OVCAR8 tumor growth and survival was evaluated. Mice (n=10 per group) were randomly assigned to treatment with vehicle IgG control, CD5L antibody, bevacizumab and combination of CD5L ab and bevacizumab. OVCAR8 cells (1×10⁶) were injected into mice (intra peritoneal). The treatment was started on day 8 post cell injection and continued until mice became moribund. Statistical tests were done using Log-rank (Mantel-Cox) test.

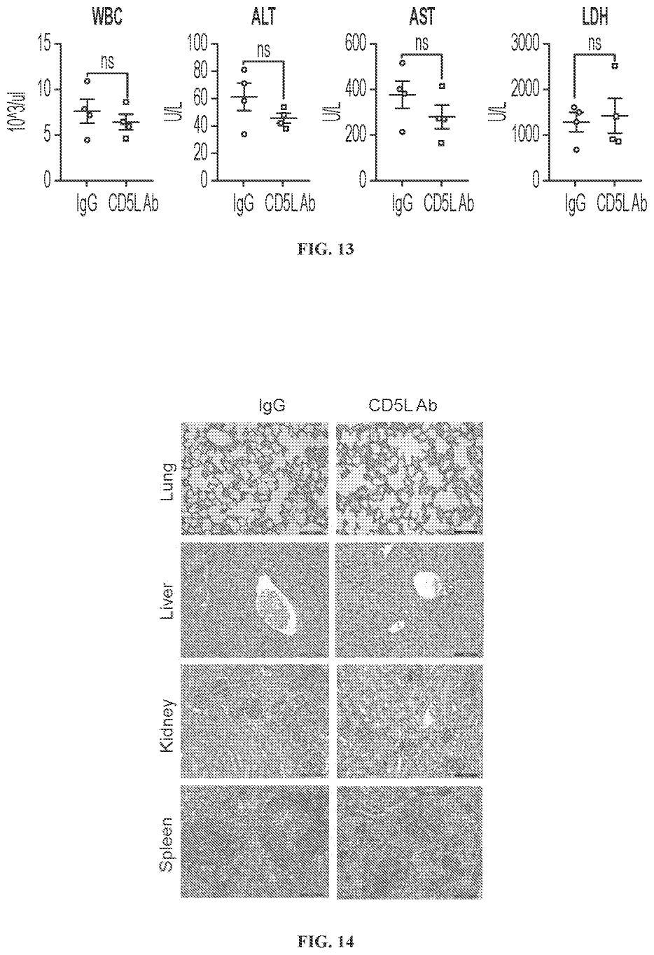

Results. As shown in FIG. 13, there was no significant difference in WBC, ALT, AST and LDH levels between CD5L antibody (R-35) treated mice and IgG control antibody treated animals. Values are expressed as means±standard error, n=4 (two-tailed t test).

H&E staining of vital organs such as lung, liver, kidney and spleen after CD5L antibody treatment did not show any differences compared to control IgG treated animals (FIG. 14). The inventors stained for blood vessels of vital organs using CD31 antibody and did not observe any significant difference between control IgG and CD5L Ab treated groups (FIG. 15).

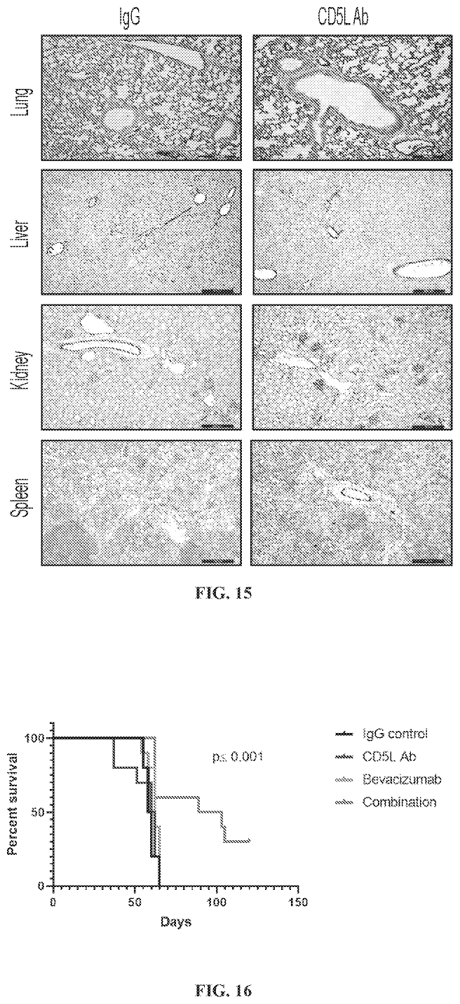

Survival was analyzed by the Kaplan-Meier method (FIG. 16). Mice treated with combination CD5L-Ab and bevacizumab had the longest overall survival (120 days) compared with those treated with control IgG (65 days), CD5L Ab alone (62 days), or bevacizumab alone (65 days) (P<0.001).

### Example 8—Humanization of CD5L-35 Antibody Sequences

Expression and purification of CD5L-35Hu mAbs. Humanized monoclonal antibodies were expressed using ExpiHEK 293 cells with each respective pair of heavy chain and light chain constructs (Table 10) in a mammalian expression vector system with human antibody constant region (IgG1) sequences.

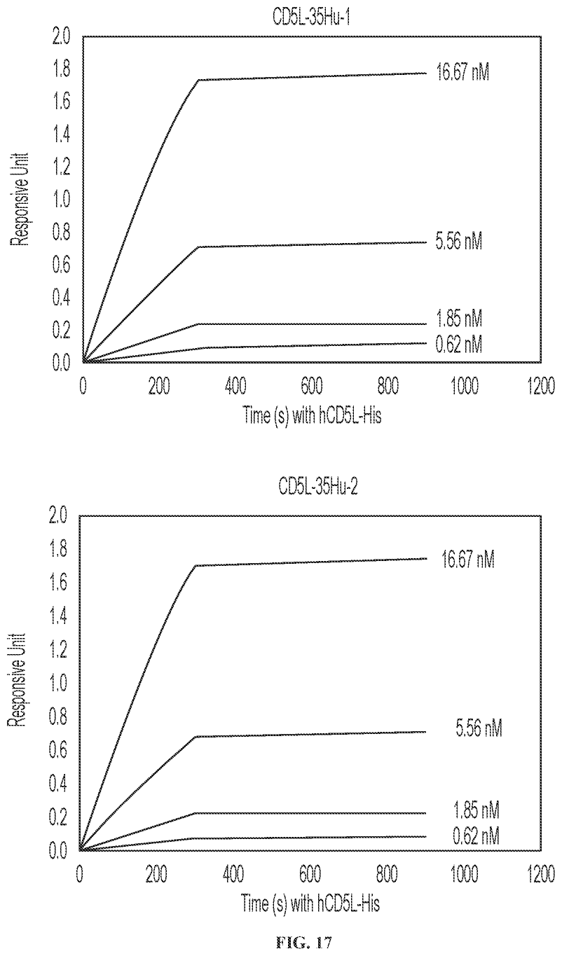

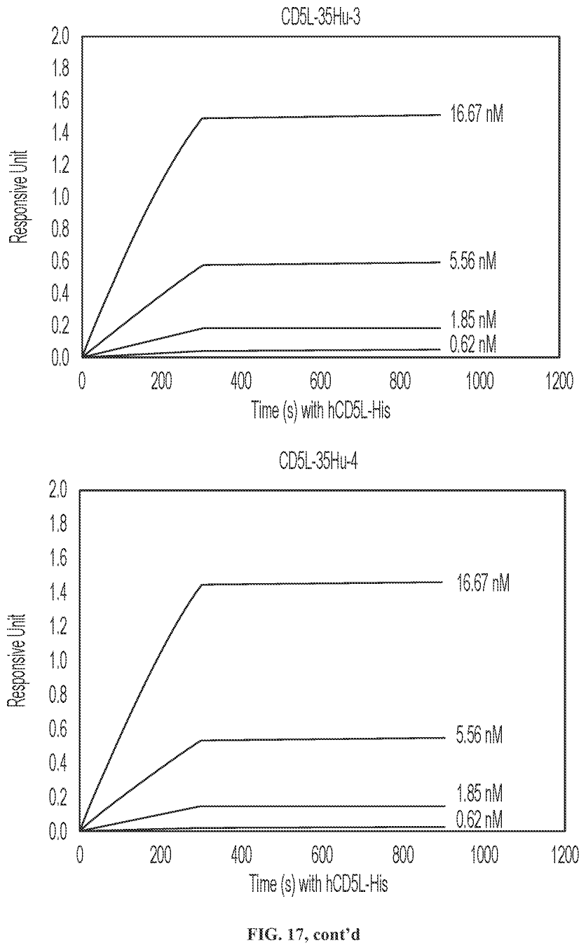

Determination of binding affinity of the humanized CD5L-35Hu mAbs to human CD5L using Octet Bilayer Interferometry (BLI) based method. Kinetic binding parameters (KD, kon, and koff) in (Table 11) were determined using Octet RED96 instrument and binding curves are shown in FIG. 17. Briefly, antibody (30 μg/ml) were loaded onto Protein A biosensors, then CD5L-His recombinantly produced protein (Sino Biologicals) were incubated with the biosensors at concentrations shown on the graphs for measuring kinetic binding rate, followed by the dissociation in kinetic buffer (ForteBio) for off rate determination. KDs are calculated using Octet software with global fitting model.

TABLE 9
Humanized CD5L-35Hu antibody variable sequences

| | | |
|---|---|---|
| CD5L- | QVQLVESGGGVVQPGRSLRLSCAASGIDLSDYYMTWVR | SEQ ID NO: 352 |
| 35Hu-H1 | QAPGKGLEYIGIIYASGNAYFASWAKGRFTISRDSSTNTLFL | |
| | QMNSLRAEDTAVYYCARNIGGSSNLWGQGTLVTVSS | |
| CD5L- | QVQLVESGGGVVQPGRSLRLSCAASGIDLSDYYMTWVR | SEQ ID NO: 353 |
| 35Hu-H2 | QAPGKGLEYIGIIYASGNAYFASWAKGRFTISRDNSTNTVF | |
| | LQMNSLRAEDTAVYYCARNIGGSSNLWGQGTLVTVSS | |
| CD5L- | AIQLTQSPSSLSASVGDRVTITCQASERIYNNLAWYQQKP | SEQ ID NO: 354 |
| 35Hu-K1 | GKAPKLLFYSASTLASGVPSRFSGSGSGTDFTLTISSLQPED | |
| | FATYYCQCTGYGTTYGLVFGGGTKVEIK | |
| CD5L- | ELQLTQTPSSLSASVGDRVTIKCQASERIYNNLAWYQQKP | SEQ ID NO: 355 |
| 35Hu-K2 | GKAPKLLFYSASTLASGVPSRFSGSGSGTDFTLTISSLQPED | |
| | FATYYCQCTGYGTTYGLVFGGGTKVEIK | |
| CD5L- | CAAGTGCAGCTGGTGGAGAGCGGCGGAGGCGTAGTGCAA | SEQ ID NO: 356 |
| 35Hu-H1 | CCCGGCAGAAGCCTGAGACTGAGCTGCGCCGCTAGCGGCA | |
| | TCGACCTGAGCGACTACTACATGACCTGGGTGAGACAAGCC | |
| | CCCGGCAAGGGCCTGGAGTACATCGGCATCATCTACGCTAG | |
| | CGGCAACGCCTACTTCGCTAGCTGGGCCAAGGGCAGATTCA | |
| | CCATCAGCAGAGACAGCAGCACCAACACCCTGTTCCTGCAGA | |
| | TGAACAGCCTGAGAGCCGAGGACACCGCCGTGTACTACTGCG | |
| | CTAGAAACATCGGCGGCAGCAGCAACCTGTGGGGCCAAGGCA | |
| | CCCTGGTGACCGTGAGCAGC | |
| CD5L- | CAAGTGCAGCTGGTGGAGAGCGGCGGAGGCGTAGTGCAACCCG | SEQ ID NO: 357 |
| 35Hu-H2 | GCAGAAGCCTGAGACTGAGCTGCGCCGCTAGCGGCATCGACCT | |
| | GAGCGACTACTACATGACCTGGGTGAGACAAGCCCCCGGCAAG | |
| | GGCCTGGAGTACATCGGCATCATCTACGCTAGCGGCAACGCCTAC | |
| | TTCGCTAGCTGGGCCAAGGGCAGATTCACCATCAGCAGAGACAAC | |
| | AGCACCAACACCGTGTTCCTGCAGATGAACAGCCTGAGAGCCGAG | |
| | GACACCGCCGTGTACTACTGCGCTAGAAACATCGGCGGCAGCAGC | |
| | AACCTGTGGGGCCAAGGCACCCTGGTGACCGTGAGCAGC | |
| CD5L- | GCCATTCAGCTGACACAGAGCCCTAGCAGCCTGAGCGCTAGCGT | SEQ ID NO: 358 |
| 35Hu-K1 | GGGCGACAGAGTGACCATCACCTGCCAAGCTAGCGAGAGAATCT | |
| | ACAACAACCTGGCCTGGTATCAGCAGAAGCCCGGCAAGGCCCCC | |
| | AAGCTGCTGTTCTACAGCGCTAGCACCCTGGCTAGCGGCGTGCCT | |
| | AGCAGATTCAGCGGCAGCGGCAGCGGCACCGACTTCACCCTGAC | |
| | CATCAGCAGCCTGCAGCCCGAGGACTTCGCCACCTACTACTGTCA | |
| | GTGCACCGGCTACGGCACCACCTACGGCCTGGTGTTCGGCGGCG | |
| | GCACCAAGGTGGAGATCAAG | |
| CD5L- | GAGCTGCAGCTGACACAGACCCCTAGCAGCCTGAGCGCTAGCGT | SEQ ID NO: 359 |
| 35Hu-K2 | GGGCGACAGAGTGACCATCAAGTGCCAAGCTAGCGAGAGAATCT | |
| | ACAACAACCTGGCCTGGTATCAGCAGAAGCCCGGCAAGGCCCCCA | |
| | AGCTGCTGTTCTACAGCGCTAGCACCCTGGCTAGCGGCGTGCCTAG | |
| | CAGATTCAGCGGCAGCGGCAGCGGCACCGACTTCACCCTGACCATC | |
| | AGCAGCCTGCAGCCCGAGGACTTCGCCACCTACTACTGTCAGTGCA | |
| | CCGGCTACGGCACCACCTACGGCCTGGTGTTCGGCGGCGGCACCAA | |
| | GGTGGAGATCAAG | |

TABLE 10
Pair-wise of light chain and heavy chain
used for CD5L-35Hu mAb expression

| Antibody name | Heavy chain | Light chain |
|---|---|---|
| CD5L-35Hu-1 | CD5L-35Hu-H1 | CD5L-35Hu-K1 |
| CD5L-35Hu-2 | CD5L-35Hu-H1 | CD5L-35Hu-K2 |
| CD5L-35Hu-3 | CD5L-35Hu-H2 | CD5L-35Hu-K1 |
| CD5L-35Hu-4 | CD5L-35Hu-H2 | CD5L-35Hu-K2 |

TABLE 11
CD5L-35Hu antibody kinetic binding
parameters determined using BLI method

| Antibody Name | KD (M) | kon(1/Ms) | kdis(1/s) | Full R\hat{ }2 |
|---|---|---|---|---|
| CD5L-35Hu-1 | <1.0E−12 | 1.46E+05 | <1.0E−07 | 0.999 |
| CD5L-35Hu-2 | <1.0E−12 | 1.25E+05 | <1.0E−07 | 0.999 |
| CD5L-35Hu-3 | <1.0E−12 | 1.19E+05 | <1.0E−07 | 1.000 |
| CD5L-35Hu-4 | <1.0E−12 | 1.06E+05 | <1.0E−07 | 1.000 |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
BERGERS & HANAHAN, 2008. Modes of resistance to anti-angiogenic therapy. Nat Rev Cancer, 8, 592-603.
BU et al., 2019. Human endotrophin as a driver of malignant tumor growth. JCI Insight, 5.
CASANOVAS et al., 2005. Drug resistance by evasion of antiangiogenic targeting of VEGF signaling in late-stage pancreatic islet tumors. Cancer Cell, 8, 299-309.
DONNINGER et al., 2004. Whole genome expression profiling of advance stage papillary serous ovarian cancer reveals activated pathways. Oncogene, 23, 8065-77.
FITZWATER & POLISKY, 1996. A SELEX primer. Methods Enzymol, 267, 275-301.
FOLKMAN, 1971. Tumor angiogenesis: therapeutic implications. N Engl J Med, 285, 1182-6.
GUIGNABERT et al., 2009. Tie2-mediated loss of peroxisome proliferator-activated receptor-gamma in mice causes PDGF receptor-beta-dependent pulmonary arterial muscularization. Am J Physiol Lung Cell Mol Physiol, 297, L1082-90.
HAEMMERLE et al., 2017. Platelets reduce anoikis and promote metastasis by activating YAP1 signaling. Nat Commun, 8, 310.
JOHN et al., 2018. A Novel Anti-LILRB4 CAR-T Cell for the Treatment of Monocytic AML. Mol Ther, 26, 2487-2495.
KUROKAWA et al., 2010. Macrophage-derived AIM is endocytosed into adipocytes and decreases lipid droplets via inhibition of fatty acid synthase activity. Cell Metab, 11, 479-92.
KUWATA et al., 2003. AIM inhibits apoptosis of T cells and NKT cells in Corynebacterium-induced granuloma formation in mice. Am J Pathol, 162, 837-47.
LAMBERTI et al., 2016. In vitro selection of RNA aptamers against CA125 tumor marker in ovarian cancer and its study by optical biosensing. Methods, 97, 58-68.
LANDEN et al., 2005. Therapeutic EphA2 gene targeting in vivo using neutral liposomal small interfering RNA delivery. Cancer Res, 65, 6910-8.
LU et al., 2010. Regulation of tumor angiogenesis by EZH2. Cancer Cell, 18, 185-97.
MAEHARA et al., 2014. Circulating AIM prevents hepatocellular carcinoma through complement activation. Cell Rep, 9, 61-74.
MIYAZAKI et al., 1999. Increased susceptibility of thymocytes to apoptosis in mice lacking AIM, a novel murine macrophage-derived soluble factor belonging to the scavenger receptor cysteine-rich domain superfamily. J Exp Med, 189, 413-22.
NARAYANA et al., 2009. Antiangiogenic therapy using bevacizumab in recurrent high-grade glioma: impact on local control and patient survival. Journal of Neurosurgery, 110, 173-180.
NOH et al., 2017. Differential Effects of EGFL6 on Tumor versus Wound Angiogenesis. Cell Rep, 21, 2785-2795.
NORDEN et al., 2008. Bevacizumab for recurrent malignant gliomas—Efficacy, toxicity, and patterns of recurrence. Neurology, 70, 779-787.
PRADEEP et al., 2015. Erythropoietin Stimulates Tumor Growth via EphB4. Cancer Cell, 28, 610-622.
QU et al., 2009. Myeloid-specific expression of Api6/AIM/Sp alpha induces systemic inflammation and adenocarcinoma in the lung. J Immunol, 182, 1648-59.
SANJURJO et al., 2015. AIM/CD5L: a key protein in the control of immune homeostasis and inflammatory disease. J Leukoc Biol, 98, 173-84.
SILVERSTEIN & FEBBRAIO, 2009. CD36, a scavenger receptor involved in immunity, metabolism, angiogenesis, and behavior. Sci Signal, 2, re3.
STOLTENBURG et al., 2015. In vitro Selection and Interaction Studies of a DNA Aptamer Targeting Protein A. PLoS One, 10, e0134403.
THAKER et al., 2006. Chronic stress promotes tumor growth and angiogenesis in a mouse model of ovarian carcinoma. Nat Med, 12, 939-44.
WEIS & CHERESH, A. 2011. Tumor angiogenesis: molecular pathways and therapeutic targets. Nat Med, 17, 1359-70.

1. A monoclonal antibody or antibody fragment, wherein the antibody or antibody fragment is characterized by clone-paired heavy and light chain CDR sequences from Tables 2 and 3.

2. The monoclonal antibody or antibody fragment of claim 1, wherein said antibody or antibody fragment is encoded by light and heavy chain variable sequences according to clone-paired sequences from Table 5.

3. The monoclonal antibody or antibody fragment of claim 1, wherein said antibody or antibody fragment is encoded by light and heavy chain variable sequences having at least 70%, 80%, 90%, or 95% identity to clone-paired sequences from Table 5.

4. (canceled)

5. The monoclonal antibody or antibody fragment of claim 1, wherein said antibody or antibody fragment comprises light and heavy chain variable sequences according to clone-paired sequences from Table 4.

6. The monoclonal antibody or antibody fragment of claim 1, wherein said antibody or antibody fragment comprises light and heavy chain variable sequences having at least 70%, 80%, 90%, or 95% identity to clone-paired variable sequences from Table 4.

7. (canceled)

8. The monoclonal antibody or antibody fragment of claim 1, wherein the antibody or antibody fragment comprises:
a heavy chain variable region (VH) comprising a VHCDR1 amino acid sequence of SEQ ID NO: 81, a VHCDR2 amino acid sequence of SEQ ID NO: 82, and a VHCDR3 amino acid sequence of SEQ ID NO: 84; and a light chain variable region (VL) comprising a VLCDR1 amino acid sequence of SEQ ID NO: 4, a VLCDR2 amino acid sequence of SEQ ID NO: 2, and a VLCDR3 amino acid sequence of SEQ ID NO: 5;
a heavy chain variable region (VH) comprising a VHCDR1 amino acid sequence of SEQ ID NO: 87, a VHCDR2 amino acid sequence of SEQ ID NO: 88, and a VHCDR3 amino acid sequence of SEQ ID NO: 89; and a light chain variable region (VL) comprising a VLCDR1 amino acid sequence of SEQ ID NO: 1, a VLCDR2 amino acid sequence of SEQ ID NO: 2, and a VLCDR3 amino acid sequence of SEQ ID NO: 6;
a heavy chain variable region (VH) comprising a VHCDR1 amino acid sequence of SEQ ID NO: 160, a VHCDR2 amino acid sequence of SEQ ID NO: 161, and a VHCDR3 amino acid sequence of SEQ ID NO: 162; and a light chain variable region (VL) comprising a VLCDR1 amino acid sequence of SEQ ID NO: 67, a VLCDR2 amino acid sequence of SEQ ID NO: 10, and a VLCDR3 amino acid sequence of SEQ ID NO: 68; or
a heavy chain variable region (VH) comprising a VHCDR1 amino acid sequence of SEQ ID NO: 178, a VHCDR2 amino acid sequence of SEQ ID NO: 179, and a VHCDR3 amino acid sequence of SEQ ID NO: 180; and a light chain variable region (VL) comprising a VLCDR1 amino acid sequence of SEQ ID NO: 77, a VLCDR2 amino acid sequence of SEQ ID NO: 10, and a VLCDR3 amino acid sequence of SEQ ID NO: 78.

9. The monoclonal antibody or antibody fragment of claim 8, wherein said antibody or antibody fragment is encoded by a heavy chain variable sequence having at least 70%, 80%, 90%, or 95% identity to SEQ ID NO: 266 and a light chain variable sequence having at least 70%, 80%, or 90% identity to SEQ ID NO: 267.

10. (canceled)

11. The monoclonal antibody or antibody fragment of claim 8, wherein said antibody or antibody fragment is encoded by a heavy chain variable sequence according to SEQ ID NO: 266 and a light chain variable sequence according to SEQ ID NO: 267.

12. The monoclonal antibody or antibody fragment of claim 8, wherein said antibody or antibody fragment comprises a heavy chain variable sequence having at least 70%, 80%, 90%, or 95% identity to SEQ ID NO: 185 and a light chain variable sequence having at least 70%, 80%, 90%, or 95% identity to SEQ ID NO: 186.

13. (canceled)

14. The monoclonal antibody or antibody fragment of claim 8, wherein said antibody or antibody fragment comprises a heavy chain variable sequence having a sequence according to SEQ ID NO: 185 and a light chain variable sequence having a sequence according to SEQ ID NO: 186.

15. The monoclonal antibody or antibody fragment of claim 8, wherein said antibody or antibody fragment is encoded by a heavy chain variable sequence having at least 70%, 80%, 90%, or 95% identity to SEQ ID NO: 268 and a light chain variable sequence having at least 70%, 80%, 90%, or 95% identity to SEQ ID NO: 269.

16. (canceled)

17. The monoclonal antibody or antibody fragment of claim 8, wherein said antibody or antibody fragment is encoded by a heavy chain variable sequence according to SEQ ID NO: 268 and a light chain variable sequence according to SEQ ID NO: 269.

18. The monoclonal antibody or antibody fragment of claim 8, wherein said antibody or antibody fragment comprises a heavy chain variable sequence having at least 70%, 80%, 90%, or 95% identity to SEQ ID NO: 188 and a light chain variable sequence having at least 70%, 80%, 90%, or 95% identity to SEQ ID NO: 189.

19. (canceled)

20. The monoclonal antibody or antibody fragment of claim 8, wherein said antibody or antibody fragment comprises a heavy chain variable sequence having a sequence according to SEQ ID NO: 188 and a light chain variable sequence having a sequence according to SEQ ID NO: 189.

21. The monoclonal antibody or antibody fragment of claim 8, wherein said antibody or antibody fragment is encoded by a heavy chain variable sequence having at least 70%, 80%, 90%, or 95% identity to SEQ ID NO: 324 and a light chain variable sequence having at least 70%, 80%, 90%, or 95% identity to SEQ ID NO: 325.

22. (canceled)

23. The monoclonal antibody or antibody fragment of claim 8, wherein said antibody or antibody fragment is encoded by a heavy chain variable sequence according to SEQ ID NO: 324 and a light chain variable sequence according to SEQ ID NO: 325.

24. The monoclonal antibody or antibody fragment of claim 8, wherein said antibody or antibody fragment comprises a heavy chain variable sequence having at least 70%, 80%, 90%, or 95% identity to SEQ ID NO: 244 and a light chain variable sequence having at least 70%, 80%, 90%, or 95% identity to SEQ ID NO: 245.

25. (canceled)

26. The monoclonal antibody or antibody fragment of claim 8, wherein said antibody or antibody fragment comprises a heavy chain variable sequence having a sequence according to SEQ ID NO: 244 and a light chain variable sequence having a sequence according to SEQ ID NO: 245.

27. The monoclonal antibody or antibody fragment of claim 8, wherein said antibody or antibody fragment is encoded by a heavy chain variable sequence having at least 70%, 80%, 90%, or 95% identity to SEQ ID NO: 338 and a light chain variable sequence having at least 70%, 80%, 90%, or 95% identity to SEQ ID NO: 339.

28. (canceled)

29. The monoclonal antibody or antibody fragment of claim 8, wherein said antibody or antibody fragment is encoded by a heavy chain variable sequence according to SEQ ID NO: 338 and a light chain variable sequence according to SEQ ID NO: 339.

30. The monoclonal antibody or antibody fragment of claim 8, wherein said antibody or antibody fragment comprises a heavy chain variable sequence having at least 70%, 80%, 90%, or 95% identity to SEQ ID NO: 258 and a light chain variable sequence having at least 70%, 80%, 90%, or 95% identity to SEQ ID NO: 259.

31. (canceled)

32. The monoclonal antibody or antibody fragment of claim 8, wherein said antibody or antibody fragment comprises a heavy chain variable sequence having a sequence according to SEQ ID NO: 258 and a light chain variable sequence having a sequence according to SEQ ID NO: 259.

33. The monoclonal antibody or antibody fragment of claim 8, wherein said antibody or antibody fragment is a humanized antibody, such as wherein said antibody or antibody fragment comprises a heavy chain variable sequence having a sequence according to SEQ ID NO: 352 and a light chain variable sequence having a sequence according to SEQ ID NO: 354; a heavy chain variable sequence having a sequence according to SEQ ID NO: 352 and a light chain variable sequence having a sequence according to SEQ ID NO: 355; a heavy chain variable sequence having a sequence according to SEQ ID NO: 353 and a light chain variable sequence having a sequence according to SEQ ID NO: 354; or a heavy chain variable sequence having a sequence according to SEQ ID NO: 353 and a light chain variable sequence having a sequence according to SEQ ID NO: 355.

34. The monoclonal antibody or antibody fragment of claim 8, wherein the antibody fragment is an IgG antibody fragment, a monovalent scFv (single chain fragment variable) antibody, divalent scFv, Fab fragment, F(ab′)₂ fragment, F(ab′)₃ fragment, Fv fragment, or single chain antibody.

35. The monoclonal antibody or antibody fragment of claim 8, wherein said antibody is an IgG antibody, a chimeric antibody or bispecific antibody.

36. (canceled)

37. The monoclonal antibody or antibody fragment of claim 8, wherein the antibody or antibody fragment is conjugated or fused to an imaging agent or a cytotoxic agent.

38. A monoclonal antibody or an antigen binding fragment thereof, which competes for binding to the same epitope as the monoclonal antibody or an antigen-binding fragment thereof according to claim 8.

39. The monoclonal antibody or antibody fragment of claim 38, wherein said antibody or antibody fragment is a humanized antibody or antibody fragment.

40. The monoclonal antibody or antibody fragment of claim 38, wherein the antibody fragment is an IgG antibody fragment, a monovalent scFv (single chain fragment variable) antibody, divalent scFv, Fab fragment, F(ab′)₂ fragment, F(ab′)₃ fragment, Fv fragment, or single chain antibody.

41. The monoclonal antibody or antibody fragment of claim 38, wherein said antibody is an IgG antibody, a chimeric antibody or bispecific antibody.

42. (canceled)

43. The monoclonal antibody or antibody fragment of claim 38, wherein the antibody is conjugated or fused to an imaging agent or a cytotoxic agent.

44. A composition comprising a therapeutically effective amount of an antibody or antibody fragment of claim 8, and a pharmaceutically acceptable diluent or vehicle.

45. (canceled)

46. A hybridoma or engineered cell encoding an antibody or antibody fragment of claim 8.

47. A method of treating a patient having a cancer, the method comprising administering an effective amount of an anti-CD5L antibody or antibody fragment.

48-60. (canceled)

61. A method for the detection of CD5L comprising:
(a) incubating a sample with an anti-CD5L antibody or antibody fragment of claim 8; and
(b) measuring the binding of the anti-CD5L antibody or antibody fragment to the sample.

62. (canceled)
